(19) United States
(12) Patent Application Publication
(10) Pub. No.: US 2024/0218467 A1
(43) Pub. Date: Jul. 4, 2024

(54) MPOX CLADE II BIOMARKER PANEL

(71) Applicant: Cepheid, Sunnyvale, CA (US)

(72) Inventors: Robert Jones, Sunnyvale, CA (US); Kalyani Mangipudi, Sunnyvale, CA (US); Soumitesh Chakravorty, Santa Clara, CA (US); Burcu Ozay, Sunnyvale, CA (US)

(73) Assignee: Cepheid, Sunnyvale, CA (US)

(21) Appl. No.: 18/540,460

(22) Filed: Dec. 14, 2023

Related U.S. Application Data
(60) Provisional application No. 63/433,373, filed on Dec. 16, 2022.

(51) Int. Cl. C12Q 1/70; B01L 3/00; B01L 7/00

(52) U.S. Cl. CPC C12Q 1/701; B01L 3/502761; B01L 7/52; B01L 2200/026; B01L 2200/028; B01L 2200/04; B01L 2300/0681

(57) ABSTRACT

The present disclosure provides a set of primers and optional probes for identifying the presence of MPOX virus, Clade II, which can be included with (e.g., in at kit) or in a cartridge for automated detection of these pathogens by nucleic acid amplification. This disclosure also provides related detection methods, as well as cartridges, systems, and kits useful in such methods.

### CROSS-REFERENCE TO RELATED APPLICATIONS

This application claims the benefit of U.S. provisional application No. 63/433,373, filed Dec. 16, 2022, which is hereby incorporated by reference in its entirety.

### INCORPORATION BY REFERENCE OF SEQUENCE LISTING PROVIDED AS AN XML FILE

The instant application contains a Sequence Listing which has been submitted electronically in XML format and is hereby incorporated by reference in its entirety. Said XML copy, created on Mar. 8, 2024, is named CPHDP024US_SL.xml and is 118,729 bytes in size.

### FIELD

The present invention relates to generally to the area of detecting MPOX Clade II viruses.

### BACKGROUND

Monkeypox (MPXV or MPOX) is a viral zoonosis (a virus transmitted to humans from animals) with symptoms similar to those seen in the past in smallpox patients, although it is clinically less severe. With the eradication of smallpox in 1980 and subsequent cessation of smallpox vaccination, MPOX virus has emerged as the most important Orthopoxvirus (OPXV) for public health. MPXV infection primarily occurs in Central and West Africa, often in proximity to tropical rainforests, and has been increasingly appearing in urban areas.

MPXV an enveloped double-stranded DNA virus that belongs to the Orthopoxvirus genus of the Poxviridae family. There are two distinct genetic clades of the MPOX virus: the Congo Basin clade (Clade I) and the West African clade (Clade IIa and IIb). Clade I has historically caused more severe disease and was thought to be more transmissible, but this Clade has not been detected in the most recent global outbreak.

Human MPOX was first identified in humans in 1970 in the Democratic Republic of the Congo in a 9-month-old boy in a region where smallpox had been eliminated in 1968. Since then, most cases have been reported from rural, rainforest regions of the Congo Basin, particularly in the Democratic Republic of the Congo and human cases have increasingly been reported from across Central and West Africa. Since 1970, human cases of MPOX have been reported in 11 African countries: Benin, Cameroon, the Central African Republic, the Democratic Republic of the Congo, Gabon, Cote d′Ivoire, Liberia, Nigeria, the Republic of the Congo, Sierra Leone and South Sudan. The true burden of MPOX is not known. Cases continue to be reported.

MPOX is a disease of global public health importance as it not only affects countries in West and Central Africa, but the rest of the world. In 2003, the first MPOX outbreak outside of Africa was in the United States of America. This outbreak led to over 70 cases of MPOX in the United States. In May 2022, multiple cases of MPOX were identified in several non-endemic countries. As of 4 Oct. 2022. there were 68,998 reported cases of MPOX in 100 countries, including 25,672 cases in the United States.

### SUMMARY

Various embodiments contemplated herein may include, but need not be limited to, one or more of the following:

Embodiment 1: A set of primers and optional probe(s) for detecting and/or identifying the presence of a MPOX Clade II virus in a sample, the set comprising: at least one primer pair and optional probe that selectively hybridizes to the OPG153 gene of MPOX Clade II, the OPG183 intergenic region of MPOX Clade II or a combination thereof, of MPOX Clade II virus, wherein said set of primers and optional probe(s) selectively hybridizes to one or more conserved regions of nucleic acid from MPOX Clade II viruses.

Embodiment 2: The set of embodiment 1, wherein each primer and optional probe has less than 90%, preferably less than 85% sequence identity to an analogous region, when present, in MPOX Clade I or non-variola orthopoxviruses.

Embodiment 3: The set of embodiment 1 or embodiment 2, wherein the primers and optional probe(s) do not selectively hybridize with MPOX Clade I viruses or non-variola orthopoxviruses.

Embodiment 4: The set of any one of embodiments 1-3, further comprising an additional primer pair and optional probe for detecting and/or identifying the presence of one or more non-variola orthopoxviruses, wherein said additional primer pair and optional probe comprises at least one primer pair and optional probe that selectively hybridizes to the DNA polymerase (E9L) gene of non-variola orthopoxvirus.

Embodiment 5: The set of embodiment 4, wherein the additional primer pair and optional probe for identifying one or more non-variola orthopoxviruses selectively hybridizes to one or more conserved regions of nucleic acid from non-variola orthopoxviruses.

Embodiment 6: The set of any one of embodiments 1-5, further comprising an additional primer pair and optional probe for detecting and/or identifying the presence of one or more MPOX Clade I viruses, wherein said additional primer pair and optional probe comprises at least one primer pair and optional probe that selectively hybridizes to one or more MPOX Clade I viruses.

Embodiment 7: The set of any one of embodiments 1-6, further comprising one or more additional primer pairs and optional probe(s) for detecting and/or identifying the presence of one or more of herpes simplex virus (HSV), varicella zoster virus (VZV), and syphilis.

Embodiment 8: The set of any one of embodiments 4-6, wherein the set comprises: the at least one primer pair and probe that selectively hybridizes to the OPG153 gene of MPOX Clade II, the at least one primer pair and probe that selectively hybridizes to the intergenic region of MPOX Clade II, and the at least one primer pair and probe that selectively hybridizes to the DNA polymerase (E9L) gene of non-variola orthopoxvirus.

Embodiment 9: The set of any one of embodiments 1-8, wherein, when present, the primer and optional probe that selectively hybridizes to the OPG153 gene of MPOX Clade II comprise a sequence that is identical or complementary to at least 15 contiguous nucleotides of one or more of SEQ ID NOs: 1, 25, 26, 27, and 28: the primers and optional probe that selectively hybridizes to the OPG183 intergenic region of MPOX Clade II comprise a sequence that is identical or complementary to at least 15 contiguous nucleotides of one or more of SEQ ID NOs:2, 29, 30, 31, and 32: the primers and optional probe that selectively hybridizes to the DNA polymerase (E9L) gene of non-variola orthopoxvirus comprise a sequence that is identical or complementary to at least 15 contiguous nucleotides of one or more of SEQ ID NO: 3, 33, 34, 35, and 36.

Embodiment 10: The set of any one of embodiments 1-9, wherein at least one of the primers and optional probe(s) comprises a detectable label.

Embodiment 11: The set of any one of embodiments 1-10, wherein at least one probe, optionally each probe, of the set comprises a fluorescent dye and a quencher molecule.

Embodiment 12: The set of any one of embodiments 1-11, wherein the set further comprises a primer pair that selectively hybridizes to an exogenous control and/or an endogenous control, wherein the exogenous control is a sample processing control, and wherein the endogenous control is a sample adequacy control.

Embodiment 13: The set of any one of embodiments 1-12, wherein the set is contained within one or more cartridge(s).

Embodiment 14: The set of embodiment 13, wherein the set is contained in one cartridge.

Embodiment 15: A cartridge for detecting and/or identifying MPOX Clade II in a sample, the cartridge comprising:

a cartridge body comprising a plurality of chambers therein, wherein the plurality of chambers includes:
i) a sample chamber having at least a fluid outlet in fluidic communication with another chamber of the plurality; and
ii) an optional lysis chamber in fluidic communication with the sample chamber, optionally wherein the sample chamber and lysis chamber are the same:

a reaction vessel fluidically coupled to the plurality of chambers of the cartridge body and configured for:
i) amplification of nucleic acid and
ii) detection and identification of one or more amplification products via real-time PCR, melt curve analysis, or a combination thereof:

a filter disposed in a fluidic path between the lysis chamber and the reaction vessel, and a set of primers and/or probes according to any one of embodiments 1-11, for identifying the presence of MPOX Clade II.

Embodiment 16: The cartridge of embodiment 15, wherein the lysis chamber comprises one or more lysis reagents for releasing nucleic acid.

Embodiment 17: The cartridge of any one of embodiments 15-16, wherein the sample chamber and the lysis chamber are the same.

Embodiment 18: The cartridge of any one of embodiments 15-17, wherein the at least one of the plurality of chambers comprises the set primers and/or probes, or subset thereof, and at least one different chamber of the plurality of chambers comprises one or more lysis reagents for releasing nucleic acid from a sample.

Embodiment 19: The cartridge of any one of embodiments 15-18, wherein the reaction vessel comprises one or more reaction chambers for detection of the plurality of amplification products.

Embodiment 20: The cartridge of any one of embodiments 15-19, wherein the reaction vessel comprises one reaction chamber.

Embodiment 21: The cartridge of embodiment 19 or 20, wherein each reaction chamber is configured to detect a single amplification product.

Embodiment 22: The cartridge of embodiment 19 or 20, wherein each reaction chamber is configured to detect a plurality of amplification products.

Embodiment 23: The cartridge of any one of embodiments 15-22, wherein the cartridge is configured to carry out isothermal amplification.

Embodiment 24: The cartridge of any one of embodiments 15-23, wherein the cartridge is configured to carry out non-isothermal amplification, optionally by thermal cycling or temperature oscillation.

Embodiment 25: The cartridge of any one of embodiments 15-24, wherein the cartridge is a Clinical Laboratory Improvement Amendments (CLIA)-compliant cartridge.

Embodiment 26: A method for detecting and identifying MPOX Clade II in a sample, the method comprising:
a) contacting nucleic acid from the sample with a set of primers and optional probe(s) according to any one of embodiments 1-12;
b) subjecting the nucleic acid, primers and optional probe(s) to amplification conditions, optionally followed by a melt curve assay:
c) detecting the presence of any amplification product(s) via real-time PCR, melt curve analysis, or a combination thereof, and
d) identifying the presence of a MPOX Clade II virus in the sample or determining that no MPOX Clade II virus detectable using the set of primers and/or probes is present, based on detection of the amplification product(s) or lack thereof, respectively.

Embodiment 27: The method of embodiment 26, wherein d) comprises differentially identifying the presence of a MPOX Clade II and/or non-variola orthopoxvirus in the sample, or determining that no MPOX Clade II and non-variola orthopoxvirus are detectable using the set of primers and/or probes present, based on detection of the amplification product(s) or lack thereof, respectively.

Embodiment 28: The method of any one of embodiments 26-27, wherein:
a) said contacting nucleic acid from the sample with the set of primers and/or probes comprises:
   placing the sample in a cartridge comprising a cartridge body having a plurality of chambers in fluidic communication, a reaction vessel having one or more reaction chambers and configured for amplification of the nucleic acid and detection of one or more amplification products, a fluidic path between the plurality of chambers and the reaction vessel, and a filter in the fluidic path; and
   if the sample comprises cells, lysing cells in the sample with one or more lysis reagents present within at least one of the plurality of chambers:
b) said subjecting the nucleic acid, primers and/or probes to amplification conditions comprises amplifying the nucleic acid with primers and/or probes present in solution within the reaction vessel.

Embodiment 29: The method of embodiment 28, wherein amplification is isothermal.

Embodiment 30: The method of embodiment 28 wherein amplification is non-isothermal, optionally, by thermal cycling or temperature oscillation.

Embodiment 31: The method of any one of embodiments 26-30, wherein said detecting is carried out in more than one reaction chambers.

Embodiment 32: The method of any one of embodiments 26-31, wherein said detecting is carried out in a single reaction chamber.

Embodiment 33: The method of any one of embodiments 26-32, wherein the sample is a skin sample, a lesion swab sample, a vesicular lesion fluid sample, a pustular lesion fluid sample, a rectal sample, a nasal aspirate sample, a nasal wash sample, a nasal swab sample, a nasopharyngeal swab sample, a saliva sample, an oropharyngeal swab sample, a throat swab sample, a bronchoalveolar lavage sample, a bronchial aspirate sample, a bronchial wash sample, an endotracheal aspirate sample, an endotracheal wash sample, a tracheal aspirate sample, a nasal secretion sample, a mucus sample, a sputum sample, a plasma sample, a whole blood sample, or a combination thereof.

Embodiment 34: The method of any one of embodiments 26-33, wherein said detecting and is done at the same facility where the sample was collected from a subject.

Embodiment 35: The method of any one of embodiments 26-34, wherein the method is a point-of-care method.

Embodiment 36: The method of any one of embodiments 26-35, wherein the method is carried out in a hospital, an urgent care center, an emergency room, a physician's office, a health clinic, or a home.

Embodiment 37: The method of any one of embodiments 26-36, wherein the method is a Clinical Laboratory Improvement Amendments (CLIA)-waived test.

Embodiment 38: The method of any one of embodiments 28-37, wherein the cartridge is a Clinical Laboratory Improvement Amendments (CLIA)-compliant cartridge, is operated in compliance with CLIA, is operated by a CLIA-compliant laboratory, or is operated in a CLIA-compliant location.

Embodiment 39: The method of any one of embodiments 26-38, wherein the method is carried out to distinguish between a virulent pathogen and a less virulent pathogen.

Embodiment 40: The method of any one of embodiments 26-39, wherein the method is carried out to facilitate a response to a pandemic, epidemic, and/or endemic pathogen.

Embodiment 41: The set of any one of embodiments 1-14, the cartridge of any one of embodiments 15-25, or the method of any one of embodiments 26-40, wherein the cartridge facilitates and/or the method comprises detection of a MPOX Clade II virus within the sample within 60 minutes, within 45 minutes, or within 30 minutes of collecting the sample from the subject.

Embodiment 42: The set of any one of embodiments 1-14, the cartridge of any one of embodiments 15-25, or the method of any one of embodiments 26-40, wherein the cartridge facilitates and/or the method comprises detection of a MPOX Clade II virus within the sample within 60 minutes, within 45 minutes, within 30 minutes, or within 20 minutes from the time the sample is placed in a cartridge.

Embodiment 43: The set, cartridge, or method of any one of embodiments 41-42, wherein the one or more lysis reagents comprise a chaotropic agent, a chelating agent, a buffer, and a detergent.

Embodiment 44: The set, cartridge, or method of embodiment 43, wherein the chaotropic agent is selected from guanidinium thiocyanate, guanidinium hydrochloride, alkali perchlorate, alkali iodide, urea, formamide, or a combination thereof.

Embodiment 45: The set, cartridge, or method of embodiment 43 or 44, wherein the one or more lysis reagents comprise a guanidinium compound, sodium hydroxide, EDTA, a buffer, and a detergent.

Embodiment 46: The set, cartridge, or method of any one of embodiments 41-45, wherein the filter is configured to bind the nucleic acid to be analyzed.

Embodiment 47: The set, cartridge, or method of any one of embodiments 41-46, wherein the filter comprises glass fibers and optionally a polymeric binder, or the glass fibers are optionally modified with a DNA binding ligand, optionally an alkylamine, a cycloalkylamine, an alkyloxy amine, a polyamine moiety, an arylamine, an intercalating agent, a DNA groove binder, a peptide, an amino acid, a protein, or a combination thereof.

Embodiment 48: The set, cartridge, or method of any one of embodiments 41-47, wherein the filter comprises a 500 micron to 2000 microns thick glass fiber disk having a pore size of 0.2 microns to 1 micron.

Embodiment 49: The set, cartridge, or method of any one of embodiments 41-48, wherein the filter is configured to bind unwanted material and allow the nucleic acid to pass through.

Embodiment 50: The set, cartridge, or method of any one of embodiments 41-49, wherein the cartridge further comprises a binding reagent, wash reagent, eluting reagent, or a combination thereof.

Embodiment 51: The set, cartridge, or method of any one of embodiments 41-50, wherein the eluting reagent comprises ammonia or an alkali metal hydroxide.

Embodiment 52: The set, cartridge, or method of any one of embodiments 41-51, wherein the eluting reagent has a pH above about 9, above about 10, or above about 11.

Embodiment 53: The set, cartridge, or method of any one of embodiments 41-52, wherein the eluting reagent comprises a polyanion, optionally a carrageenan, a carrier nucleic acid, or i-carrageenan and KOH.

Embodiment 54: The set, cartridge, or method of any one of embodiments 41-53, wherein the reaction vessel comprises up to 4 reaction chambers.

Embodiment 55: The set, cartridge, or method of any one of embodiments 41-54, wherein the reaction vessel comprises one reaction chamber.

Embodiment 56: The set, cartridge, or method of any one of embodiments 41-55, wherein at least one of the plurality of chambers comprises one or more lyophilized reagents.

Embodiment 57: The set, cartridge, or method of embodiment 56, wherein the one or more lyophilized reagents is/are in the form of one or more beads.

Embodiment 58: The set, cartridge, or method of embodiment 56 or 57, wherein the one or more lyophilized reagents are selected from primers, probes, a salt, dNTPs, a thermostable polymerase, a reverse transcriptase, or a combination thereof.

Embodiment 59: The set, cartridge, or method of embodiment 58, wherein the one or more lyophilized reagents comprise lyophilized primers and probes.

Embodiment 60: The set, cartridge, or method of any one of embodiment 41-55, wherein reagents and components in the reaction vessel are in solution.

Embodiment 61: A system for detecting MPOX Clade II in a biological sample, the system comprising:
a module having a receiving bay for receiving the cartridge of any one of embodiments 15-25, wherein the module includes one or more mechanisms within the receiving bay for manipulating a fluid sample within the cartridge, and an instrument that interfaces with the reaction vessel; and
a memory having programmable instructions recorded thereon, that are specially configured to operate the module according to a monkepox Clade II assay protocol to determine nucleic acid sequence characteristics of MPOX Clade II.

Embodiment 62: The system of embodiment 61, wherein the module and/or system further comprises: a scanner or reader configured to read an identifier on the cartridge: wherein the instructions are configured to determine an applicable protocol based on reading or scanning of the identifier; and wherein the system operates the module according to the applicable protocol based on an input from the scanner or reader.

Embodiment 63: The system of embodiment 61 or 62, wherein the module and/or system further comprises: an enclosure: a plurality of modules that includes said module, wherein modules are substantially identical and configured to concurrently perform assays on cartridges received therein.

Embodiment 64: The system of embodiment 63, further comprising a networking platform for transmitting results derived from module operation.

### BRIEF DESCRIPTION OF THE DRAWINGS

FIGS. 1A-1C show an overview of a sample cartridge with a valve assembly configured for performing differing sample processes, including chemical lysing of targets, which is configured for PCR and optional integrated nucleic acid analysis of the MPOX Clade II biomarker panel in accordance with some embodiments of the invention. FIG. 1A shows the sample cartridge body with reaction vessel, FIG. 1B shows an exploded view of the sample cartridge, and FIG. 1C shows components of the valve assembly, in accordance with some embodiments.

FIG. 2 illustrates various valve assemblies A, B, C, D, each suited for one or more types of target lysing, any of which may be used in a respective sample cartridge.

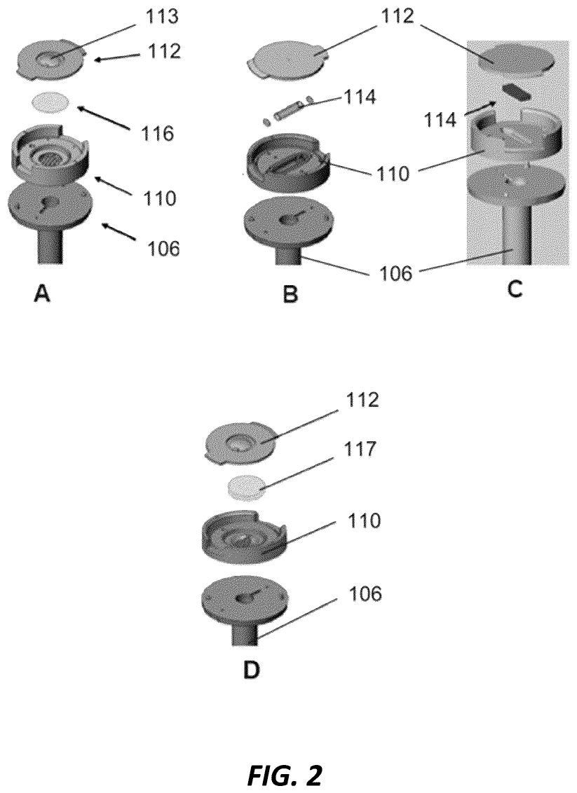

FIGS. 3A-3C show illustrative, but non-limiting, embodiments of the modules and systems (e.g., processing units) for the PCR detection and/or quantification and optional integrated nucleic acid analysis for the biomarker panel. FIG. 3A illustrates a module configured to receive and interact with the valve assembly of the cartridge to operate the cartridge to facilitate sample preparation and analysis. FIG. 3B illustrates a processing unit (e.g., analytical testing unit) of the module that interacts with the fluid sample in the reaction vessel to facilitate sample processing and analytical testing (e.g., PCR and, optionally, nucleic acid analysis) for the biomarker panel. FIG. 3C illustrates an analytical system having multiple such modules within an enclosure so as to receive multiple sample cartridges therein for testing of the biomarker panel and/or various other targets or panels.

FIG. 4 shows a non-limiting workflow for PCR and optional nucleic acid analysis (e.g., nucleic acid amplification) of the targeted assay. In some embodiments, PCR and nucleic acid analysis, when performed, are both performed on the same sample. Thus, a single sample can be introduced into one sample chamber. In other embodiments the sample may be processed differently for PCR and nucleic acid analysis for the target assay panel.

FIG. 5 illustrates an exploded view of the sample cartridge illustrating its major components, including the lid, multi-chamber body, reaction vessel, valve assembly and base, in accordance with some embodiments. The chambers may be used to perform various processes (e.g., extract, purify, deamidate, desulfonate) on the sample.

FIGS. 6A-6B show specialized components of the cartridge configured for the targeted assay panel, in accordance with some embodiments. As pictured, a specialized component is a valve assembly having a syringe tube through which the sample is injected and multiple ports on a valve body that facilitate transport of fluid sample between the respective processing chambers upon rotation of the valve assembly.

FIG. 7. MPOX Clade II is split into Clade IIa and Clade IIb. The current 2022 outbreak genomes are all from Clade IIb. FIG. 8 shows a tree from NextStrain (nextstrain.org/MPOX/mpxv) that illustrates this. Clade I corresponds to the lowest line and cluster of points. Clade II branches off from Clade I at the left side of the tree. Clade II then splits into Clade IIa (lower Clade II branch) and Clade IIB (upper Clade II branch).

FIG. 8. Ct values of each analyte.

FIG. 9. End point fluorescence (EPF) for each analyte.

FIG. 10. Test results from GeneXpert software.

FIG. 11. Fluorescence vs. cycles output graph for 50 copies/mL NATtrol MPOX Virus input.

FIG. 12. Fluorescence vs. cycles output graph for a negative sample.

### DETAILED DESCRIPTION

The present disclosure describes methods, compositions, devices, and systems that facilitate the rapid detection of MPOX Clade II viruses in a selective and specific manner that is readily automated and can be employed in point-of-care devices, and in some embodiments, enabling the identification of MPOX Clade II viruses versus Clade I viruses in a subject. The approach relies on nucleic acid amplification to detect biomarkers of Clade II, which entails contacting sample nucleic acids with primers and/or optional probes that target Clade II-specific nucleic acids (e.g., SEQ ID NOs: 1, 2, 3, below), subjecting the nucleic acid, primers, and optional probes to amplification conditions, and detecting the presence of any amplification product(s) to differentially identify the presence of Clade II versus Clade I viruses in the sample or to determine that no MPOX virus detectable using the set of primers is present in the sample.

### Definitions

Terms used in the claims and specification are defined as set forth below unless otherwise specified.

Orthopoxvirus is a genus of viruses in the family Poxviridae and subfamily Chordopoxvirinae. Vertebrates, including mammals and humans, and arthropods serve as natural hosts. There are 12 species in this genus. Diseases associated with this genus include smallpox, cowpox, horsepox, camelpox, and MPOX. The most widely known member of the genus is variola virus, which causes smallpox.

As used herein the term non-variola orthopoxvirus refers to viruses other than variola virus in the Orthopoxvirus genus.

Monkeypox virus (MPXV or MPOX virus) is an enveloped double-stranded DNA virus that belongs to the Orthopoxvirus genus of the Poxviridae family. First identified in 1958 within a group of Asian monkeys (Macaca fascicularis) and rhesus macaques (Macaca mulatta), MPOX is viral zoonotic disease capable of human-to-human transmission. Two clades of MPOX have been described and have recently undergone a nomenclature change. MPOX clade I (formerly Congo-basin) is associated with a higher rate of mortality compared to Clade II (formerly West-African). MPOX has an incubation period ranging from 5-21 days, initial symptom onset typically lasts 1-5 days and consists of fever, headache, myalgia, lymphadenopathy, and fatigue before the appearance of vesiculopustular rash lasting approximately 2-3 weeks. Human-to-human transmission occurs via direct contact with lesions or bodily fluids, through contaminated fomites, or through respiratory secretions. In the spring of 2022 several cases of MPOX, specifically MPOX Clade IIb, were identified in non-endemic areas.

The “OPG153 gene of MPOX Clade II” refers to a region in a Clade II MPOX virus that corresponds to Gene Accession No. (GenBank) NC_063383.1, position 132396-136092 (SEQ ID NO:1). In this context, the term “corresponds to” refers to a region in a Clade II MPOX virus genome that aligns with SEQ ID NO: 1, when compared and aligned for maximum correspondence, as measured using a sequence comparison algorithm or by visual inspection. In various embodiments, a corresponding region has at least 70%, 80%, 90%, 92%, 94%, 96%, 98%, or 99% or more sequence identity to SEQ ID NO:1.

The “OPG183 intergenic region of MPOX Clade II” refers to a region in a Clade II MPOX virus that corresponds to Gene Accession No. (GenBank) NC_063383.1, position 156408-156516. In this context, the term “corresponds to” refers to a region in a Clade II MPOX virus genome that aligns with SEQ ID NO:2, when compared and aligned for maximum correspondence, as measured using a sequence comparison algorithm or by visual inspection. In various embodiments, a corresponding region has at least 70%, 80%, 90%, 92%, 94%, 96%, 98%, or 99% or more sequence identity to SEQ ID NO:2.

The term “nucleic acid” refers to a nucleotide polymer, and unless otherwise limited, includes analogs of natural nucleotides that can function in a similar manner (e.g., hybridize) to naturally occurring nucleotides.

The term nucleic acid includes any form of DNA or RNA, including, for example, genomic DNA: complementary DNA (cDNA), which is a DNA representation of mRNA, usually obtained by reverse transcription of messenger RNA (mRNA) or by amplification: DNA molecules produced synthetically or by amplification: mRNA; and non-coding RNA.

The term nucleic acid encompasses double- or triple-stranded nucleic acid complexes, as well as single-stranded molecules. In double- or triple-stranded nucleic acid complexes, the nucleic acid strands need not be coextensive (i.e, a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like.

More particularly, in some embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (20020) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs), which are described in U.S. Pat. Nos. 6,794,499, 6,670,461, 6,262,490, and 6,770,748, which are incorporated herein by reference in their entirety for their disclosure of LNAs.

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

As used herein with respect to a nucleic acid sequence, the term “conserved” refers to a sequence identical or similar across multiple iterations of the sequence in nature. A given “conserved” sequence may, in different embodiments have at least 60, 65, 70, 75, 80, 85, 90, 91, 92, 93, 94, 95, 96, 97, 98, or 99% nucleotide sequence identity, or may be 100% identical across a data set. For these purposes, the percent sequence identity requirement is met where all sequences in the data set meet this requirement when aligned with a reference sequence from the data set. The data set can include sequences from a given viral strain or clade, for example, in which case, the sequence can be said to be conserved within that strain or clade. The data set can include sequences from multiple viral strains or clades, for example, in which case the sequence can be said to be conserved across those multiple viral strains or clades. A sequence can be conserved in one strain or clade, for example, and not in another strain or clade. In particular, the sequence is not conserved in a strain if it does not meet the terms of the definition set forth in this paragraph. In various embodiments, the data set can have at least 3, 5, 10, 15, 20, 25, 50, 100, 200, 300, 400, 500, 600, 700, 800, 900, 1000 or more sequences in it. For example, a sequence can be at least 90% conserved across a data set of at least 500, at least 95% conserved across a data set of at least 400, at least 95% conserved across a data set of at least 300, at least 95% conserved across a data set of at least 200, at least 96% conserved across a data set of at least 150, at least 97% conserved across a data set of at least 100, at least 98% conserved across a data set of at least 50, at least 98% conserved across a data set of at least 10, at least 98% conserved across a data set of at least 5, at least 99% conserved across a data set of at least 10, at least 99% conserved across a data set of at least 5, etc.

As used herein, a “conserved region” refers to a subsequence of a longer nucleic acid sequence that is conserved, as defined above. The length of the conserved subsequence can be at least 6, 7, 8, 9, 10, 11, 12, 13, 14, 15, 16, 17, 18, 19, 20, 21, 22, 23, 24, 25, 26, 27, 28, 29, 30, 35, 40, 45, or more nucleotides in length. In some embodiments, a conserved region can be less than 500, 400, 300, 200, or one hundred nucleotides in length. Thus, for example, a conserved region can be between 10 and 500, 15 and 400, 20 and 300, 25 and 200, or 30 and 100 nucleotides long and have at least 90, 91, 92, 93, 94, 95, 96, 97, 98, or 99% nucleotide sequence identity over this subsequence.

As used herein an “analogous region” refers to a subsequence of a longer nucleic acid sequence that aligns with a region in a reference sequence. An analogous region can, and often does, have a similar structure and/or function (e.g., analogous regions can encode a protein domain that performs the same function in two protein isoforms).

As used herein, the term “gene” encompasses coding sequences, introns, and any associated control sequences that participate in the expression of the coding sequences.

As used herein, the term “complementary” refers to the capacity for precise pairing between two nucleotides: i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

“Selective hybridization” or “selective annealing” refers to the binding of a nucleic acid to a target nucleic acid in the absence of substantial binding to other nucleic acids present in the hybridization mixture under defined stringency conditions. Those of skill in the art recognize that relaxing the stringency of the hybridization conditions allows sequence mismatches to be tolerated.

In some embodiments, hybridizations are carried out under stringent hybridization conditions. The phrase “stringent hybridization conditions” generally refers to a temperature in a range from about 5° C. to about 20° ° C. or 25° C. below than the melting temperature (Tₘ) for a specific sequence at a defined ionic strength and pH. As used herein, the Tₘ is the temperature at which a population of double-stranded nucleic acid molecules becomes half-dissociated into single strands. Methods for calculating the Tₘ of nucleic acids are well known in the art (see, e.g., Berger and Kimmel (1987) METHODS IN ENZYMOLOGY, VOL. 152: GUIDE TO MOLECULAR CLONING TECHNIQUES, San Diego: Academic Press, Inc. and Sambrook et al. (1989) MOLECULAR CLONING: A LABORATORY MANUAL, 2ND ED., VOLS. 1-3, Cold Spring Harbor Laboratory), both incorporated herein by reference for their descriptions of stringent hybridization conditions). As indicated by standard references, a simple estimate of the Tₘ value may be calculated by the equation: Tₘ=81.5+0.41(% G+C), when a nucleic acid is in aqueous solution at 1 M NaCl (see, e.g., Anderson and Young, Quantitative Filter Hybridization in NUCLEIC ACID HYBRIDIZATION (1985)). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of at least about 60° C. and a salt concentration of about 0.2 molar at pH7. Tₘ calculation for oligonuclotide sequences based on nearest-neighbors thermodynamics can carried out as described in “A unified view of polymer, dumbbell, and oligonucleotide DNA nearest-neighbor thermodynamics” John SantaLucia, Jr., PNAS Feb. 17, 1998 vol. 95 no. 4 1460-1465 (which is incorporated by reference herein for this description).

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules.

The term “primer” refers to an oligonucleotide that is capable of hybridizing (also termed “annealing”) with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least 7 nucleotides long and, in some embodiments, range from 10 to 30 nucleotides, or, in some embodiments, from 10 to 60 nucleotides, in length. In some embodiments, primers can be, e.g., 15 to 50 nucleotides long. Short primer molecules generally require cooler temperatures to form sufficiently stable hybrid complexes with the template. A primer need not reflect the exact sequence of the template but must be sufficiently complementary to hybridize with a template.

A primer is said to “anneal to” or “hybridize to” another nucleic acid if the primer, or a portion thereof, hybridizes to a nucleotide sequence within the nucleic acid. The statement that a primer hybridizes to a particular nucleotide sequence is not intended to imply that the primer hybridizes either completely or exclusively to that nucleotide sequence. For example, in some embodiments, amplification primers used herein are said to “anneal to” or be “specific for” a nucleotide sequence.” This description encompasses primers that anneal wholly to the nucleotide sequence, as well as primers that anneal partially to the nucleotide sequence.

The term “primer pair” refers to a set of primers including a 5′ “upstream primer” or “forward primer” that hybridizes with the complement of the 5′ end of the DNA sequence to be amplified and a 3′ “downstream primer” or “reverse primer” that hy bridizes with the 3′ end of the sequence to be amplified. As will be recognized by those of skill in the art, the terms “upstream” and “downstream” or “forward” and “reverse” are not intended to be limiting, but rather provide illustrative orientations in some embodiments.

A “probe” is a nucleic acid capable of binding to a target nucleic acid of complementary sequence through one or more types of chemical bonds, generally through complementary base pairing, usually through hydrogen bond formation, thus forming a duplex structure. The probe can be labeled with a detectable moiety to permit facile detection of the probe, particularly once the probe has hybridized to its complementary target. Alternatively, however, the probe may be unlabeled, but may be detectable by specific binding with a ligand that is labeled, either directly or indirectly. Probes can vary significantly in size.

As used herein with reference to a portion of a primer or a nucleotide sequence within the primer, the term “specific for” a nucleic acid, refers to a primer or nucleotide sequence that can specifically anneal to the target nucleic acid under suitable annealing conditions.

The term “target” is used herein with reference to “target nucleic acids,” as well as “target organisms.” The former refers to nucleic acids to be detected, and the latter refers to organisms to be detected. The term, “target nucleic acid” is generally used herein to refer to a segment of nucleic acid that is defined by a primer pair and that gives rise to an amplicon produced in an amplification reaction: the term “amplification target” is also used herein to refer to this type of target nucleic acid. Primers and probes are also said to “target” nucleic acid sequences, and so these sequences can also be understood as “target nucleic acids.” Additionally, primers and probes are said to “target” or “be specific for” genes. In this usage, the primers and probes can be used to detect the presence of a particular gene by specifically hybridizing to a portion of the gene that indicates its presence. The meaning of “target” and “target nucleic acids” will be clear to one of skill in the art from the context in which the term is employed. In some embodiments, multiple target nucleic acids can be detected to detect a single target organism. In some embodiments, a single target nucleic acid can be detected to detect a single target organism. In some embodiments, an assay can employ multiple target nucleic acids for one or more target organisms and single target nucleic acids for one or more different target organisms.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include PCR, nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), helicase-dependent amplification (HDA), and the like. Descriptions of such techniques can be found in, among other sources, Ausubel et al.: PCR Primer: A Laboratory Manual, Diffenbach, Ed., Cold Spring Harbor Press (1995): The Electronic Protocol Book, Chang Bioscience (2002): Msuih et al., J. Clin. Micro. 34:501-07 (1996): The Nucleic Acid Protocols Handbook, R. Rapley, ed., Humana Press, Totowa, N.J. (2002): Abramson et al., Curr Opin Biotechnol. 1993 Feb.: 4(1):41-7. U.S. Pat. No. 6,027,998: U.S. Pat. No. 6,605,451, Barany et al., PCT Publication No. WO 97/31256: Wenz et al., PCT Publication No. WO 01/92579; Day et al., Genomics, 29(1): 152-162 (1995), Ehrlich et al., Science 252:1643-50 (1991): Innis et al., PCR Protocols: A Guide to Methods and Applications, Academic Press (1990): Favis et al., Nature Biotechnology 18:561-64 (2000); and Rabenau et al., Infection 28:97-102 (2000): Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-): LCR Kit Instruction Manual, Cat. #200520, Rev. #050002, Stratagene, 2002: Barany, Proc. Natl. Acad. Sci. USA 88:188-93 (1991): Bi and Sambrook, Nucl. Acids Res. 25:2924-2951 (1997): Zirvi et al., Nucl. Acid Res. 27:e40i-viii (1999); Dean et al., Proc Natl Acad Sci USA 99:5261-66 (2002): Barany and Gelfand, Gene 109:1-11 (1991); Walker et al., Nucl. Acid Res. 20:1691-96 (1992): Polstra et al., BMC Inf. Dis. 2:18-(2002): Lage et al., Genome Res. 2003 Feb.: 13(2):294-307, and Landegren et al., Science 241:1077-80 (1988), Demidov, V., Expert Rev Mol Diagn. 2002 Nov.: 2(6):542-8., Cook et al., J Microbiol Methods. 2003 May:53(2): 165-74, Schweitzer et al., Curr Opin Biotechnol. 2001 Feb.: 12(1):21-7, U.S. Pat. Nos. 5,830,711, 6,027,889, 5,686,243, PCT Publication No. WO0056927A3, and PCT Publication No. WO9803673A1.

In some embodiments, amplification comprises at least one cycle of the sequential procedures of: annealing at least one primer with complementary or substantially complementary sequences in at least one target nucleic acid: synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

As used herein, the term “amplification conditions” refers to conditions that promote amplification of a target nucleic acid in the presence of suitable primers.

As used herein, “in solution” means not immobilized on a substrate of any kind, for example, a bead or a surface in a cassette, such as a chamber wall.

A “multiplex amplification reaction” is one in which two or more nucleic acids distinguishable by sequence are amplified simultaneously.

The term “qPCR” is used herein to refer to quantitative real-time polymerase chain reaction (PCR), which is also known as “real-time PCR” or “kinetic polymerase chain reaction:” all terms refer to PCR with real-time signal detection.

The term “melt curve analysis” refers to the use of the dissociation characteristics of a segment of double-stranded nucleic during heating. Originally, strand dissociation was observed using UV absorbance measurements, but techniques based on fluorescence measurements are now the most common approach. The temperature-dependent dissociation between two DNA-strands can be measured in a “melt assay,” for example, using a DNA-intercalating fluorophore, such as SYBR green or EvaGreen, or fluorophore-labelled DNA probes. In the case of SYBR green (which fluoresces 1000-fold more intensely while intercalated in the minor groove of two strands of DNA), the dissociation of the DNA during heating is measurable by the large reduction in fluorescence that results. Alternatively, juxtapositioned probes (one featuring a fluorophore and the other, a suitable quencher) can be used to determine the complementarity of the probe to the target nucleic acid sequence.

A “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, template nucleic acid, nucleotides, labels, dyes, nucleases, dNTPs, and the like. Reagents for enzyme reactions include, for example, substrates, cofactors, buffer, metal ions, inhibitors, and activators.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “dye,” as used herein, generally refers to any organic or inorganic molecule that absorbs electromagnetic radiation and produces a detectable signal (e.g., a fluorescent signal).

The term “quencher,” as used herein generally refers to any organic or inorganic molecule that reduces the level of a detectable signal.

As used herein, the term “detecting” refers to “determining the presence of” an item, such as a nucleic acid sequence, e.g., one that is indicative of the presence of a MPOX virus. Detection can include the determination of the presence of a MPOX virus, without definitive identification of that MPOX virus: the determination of the presence of one or more MPOX virus belonging to a MPOX virus clade: the determination of the presence of a particular, known MPOX virus strain; or determination of the presence of a novel (not previously described) MPOX virus or other orthopoxvirus strain.

As used herein, the term “treatment regimen” refers to any medical intervention intended to mitigate the symptoms and/or the pathology of a disorder. The treatment regimen can include one or more actions (e.g., bed rest, increasing fluid intake), non-prescription or prescription medications, supplements, foods, drinks, or the use of medical devices (e.g., a respirator).

As used herein, “Clinical Laboratory Improvement Amendments (CLIA)” refers to The Clinical Laboratory Improvement Amendments of 1988 (CLIA) regulations in effect as of the original filing date of the present application. The CLIA regulations include federal standards applicable to all U.S. facilities or sites that test human specimens for health assessment or to diagnose, prevent, or treat disease. A “CLIA-compliant” test is one that complies with these regulations. “CLIA-waived” tests include tests that does not comply with all of these regulations. For example, CLIA-waived tests include test systems cleared by the U.S. Food and Drug Administration for home use and those tests approved for waiver under the CLIA criteria.

As used herein, the term “virulent” can refer to the degree of infectivity of, and/or the severity of disease induced by, a pathogen, such as a virus. A “more virulent” virus is more infective and/or induces more severe disease than a reference virus and vice versa. In many embodiments, a “less virulent” virus does not typically induce disease requiring hospitalization.

An “endogenous control,” as used herein refers to a moiety that is naturally present in the sample to be used for detection. In some embodiments, an endogenous control is a “sample adequacy control” (SAC), which may be used to determine whether there was sufficient sample used in the assay, or whether the sample comprised sufficient biological material, such as cells. In some embodiments, an endogenous control is an RNA (such as an mRNA, RNA, ribosomal RNA, etc.), such as a human RNA for a human sample. Nonlimiting exemplary endogenous controls include ABL mRNA, GUSB mRNA, GAPDH mRNA, TUBB mRNA, and UPK1a mRNA. In some embodiments, an endogenous control, such as an SAC, is selected that can be detected in the same manner as the target nucleic acid (e.g., RNA) is detected and, in some embodiments, simultaneously with the target nucleic acid (e.g., RNA).

An “exogenous control,” as used herein, refers to a moiety that is added to a sample or to an assay, such as a “sample processing control” (SPC). In some embodiments, an exogenous control is included with the assay reagents. An exogenous control is typically selected that is not expected to be present in the sample to be used for detection, or is present at very low levels in the sample such that the amount of the moiety naturally present in the sample is either undetectable or is detectable at a much lower level than the amount added to the sample as an exogenous control. In some embodiments, an exogenous control comprises a nucleotide sequence that is not expected to be present in the sample type used for detection of the target nucleic acid (e.g., RNA). In some embodiments, an exogenous control comprises a nucleotide sequence that is not known to be present in the species from whom the sample is taken. In some embodiments, an exogenous control comprises a nucleotide sequence from a different species than the subject from whom the sample was taken. In some embodiments, an exogenous control comprises a nucleotide sequence that is not known to be present in any species. In some embodiments, an exogenous control is selected that can be detected in the same manner as the target nucleic acid (e.g., RNA) is detected and, in some embodiments, simultaneously with the target nucleic acid (e.g., RNA). In some embodiments, the exogenous control is an RNA. In some such embodiments, the exogenous control is an Armored RNAR, which comprises RNA packaged in a bacteriophage protective coat. See, e.g., WalkerPeach et al, Clin. Chem. 45: 12: 2079-2085 (1999).

### MPOX Clade II and Other Orthopoxvirus Biomarker Targets

In various embodiments, MPOX Clade II viruses can be detected by nucleic acid amplification in a monkey pox (MPX or MPOX) panel assay, which can include detection of MPX Clade II viruses and/or non-variola orthopoxviruses. In certain embodiments, the monkey pox panel assay can detect MPOX Clade I viruses and/or non-variola orthopoxviruses by nucleic acid amplification.

In some embodiments, the viruses discussed above can be detected by nucleic acid amplification in a MPX Clade II panel assay, for example, in multiplex amplification reactions, which can be designed to detect 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more target nucleic acids per amplification reaction mixture. This degree of multiplexing can be achieved by using primers and probes that do not substantially cross-react or bind off-target and that can reliably identify the pathogens they target in the face of antigenic drift. Bioinformatic analysis of multiple databases can be carried out to identify primers and probes for highly conserved regions in the genomes of these pathogens.

Recognized herein are various issues with currently available multiplexed PCR methods. For instance, while multiplexing a large number of target amplification reactions (e.g., multiplexed PCR) may be possible, it is not straightforward to detect multiple amplicons simultaneously. So far, multiplexed q-PCR methods, defined as the processes by which one amplifies and detects a plurality of nucleic acid sequences simultaneously in a single reaction chamber, has been implemented for a small number of amplicons. It is of great interest to efficiently multiplex the assays in the same reaction volume and allow for multiple concurrent target amplification and detection in the same reaction chamber. Such an approach may not only better utilize the original DNA sample, but also significantly reduce any complexities associated with the fluidics and liquid-handling procedures for running multiple single-plex reactions.

Attempts at creating multiplexed q-PCR methods have been plagued by practical issues of simultaneously detecting different nucleic acid sequences in a single sample. One approach is to associate different reporter molecules (e.g., fluorescent dyes) to individual amplicons during the PCR reaction which enables parallel detection of individual reporters by different “colors”. While such approach, in theory, may offer parallelism, it is limited by: (i) the number of different reporter molecules available: (ii) crosstalk optical signal present in a channel due to the optical signal in an adjacent channel; and (iii) the availability of imagers and detectors capable of differentiating different signals. Another possible approach to offer multiplexing capability is to divide the biological sample of interest and physically place it, using fluidic systems, into separate, single and isolated amplification chambers. While this approach may effectively create multiplex q-PCR by performing multiple single-plex (i.e., one amplicon per chamber) q-PCR reactions, it may be suboptimal, since it may reduce the number of target nucleic acid sequences in each chamber which may create stochastic anomalies (Poisson noise) in the acquired data when the original sample has a small concentration. Further, it requires complex fluidic handling procedures.

Highly multiplexed detection of DNA sequences in a sample may be done through adopting analytical platforms such as DNA microarrays or next-generation DNA sequencers, but not q-PCR or equivalent. Microarrays, in particular, are massively-parallel, affinity-based biosensors where target nucleic acids are captured selectively from the same sample at different addressable coordinates (e.g., pixels) on a solid surface. Each addressable coordinate can have a unique capturing DNA or RNA probe, complementary to a target nucleic acid sequence to be detected in the sample. While microarrays may offer high multiplexing capability, they are semiquantitative and are inferior in terms of limit-of-detection (LOD) and detection dynamic range (DDR), due to their end-point detection nature (i.e., no real-time detection) and the fact that they lack any target amplification.

Due to the range of targets for primers and probes and target amplicons, a multiplex strategy was followed to screen and select the primers and oligos. The MPX Clade II Panel multiplex design strategy can involve the following steps: singleplex design for all primers/probes, multiplex with background oligos and matrix for primers/probes, multiplex with primers and probes divided in different pools, sequencing of samples, re-design of primers/probes if required, multiplex with all panel primers/probes together, and repeat one or more steps when required.

In addition, to increase the number of target nucleic acids detected per channel, the following approaches can be used: (i) Taqman and melt probes can be combined in the same channel using melt probe with a Tₘ below annealing temperature (no amplification curve); and (ii) several melt target nucleic acids in one channel: the melt window for each target will be dependent on the sequence variation of the target.

Due to the high mutation rate of the viral target organisms, which made it difficult to find conserved regions, amplification detection was preferred to avoid a large variation in the melt probes and Tₘ windows associated with the mutations.

Because a large number of oligonucleotides combined in one reaction mixture can lead to unwanted interactions between them, in some embodiments, modified nucleotides can be used to reduce primer-primer interactions.

Some primers and probes for non-variola orthopoxviruses (including MPX) are known and include, for example, those shown in Table 1 below.

TABLE 1
Primers and Probes for MPOX Virus or Orthopoxvirus
detection (adopted from Wu F., medRxiv, 2022/Aug./
10, 22278644)

| | | | SEQ |
|---|---|---|---|
| | | | ID |
| Target | Oligo | Oligo Sequence (5′ to 3′) | NO |
| MPV_O2L | Forward | TAGTGAGTTCGGCGACAAAG | 4 |
| | Reverse | GTATCGCATCTCTCGGGTATTC | 5 |
| | Probe | ACCGGTAATCTTGTCGAGGAGGACA | 6 |
| MPV_F3L | Forward | CATCTATTATAGCATCAGCATCAGA | 7 |
| | Reverse | GATACTCCTCCTCGTTGGTCTAC | 8 |
| | Probe | TGTAGGCCGTGTATCAGCATCCATT | 9 |
| MPV_C3L | Forward | TGTCTACCTGGATACAGAAAGCAA | 10 |
| | Reverse | GGCATCTCCGTTTAATACATTGAT | 11 |
| | Probe | CCCATATATGCTAAATGGTACCGGTAC | 12 |
| | | CGGA | |
| MPV_G2R_ | Forward | CACACCGTCTCTTCCACAGA | 13 |
| WA | Reverse | GATACAGGTTAATTTCCACATCG | 14 |
| | Probe | AACCCGTCGTAACCAGCAATACATTT | 15 |
| OPV_B2R | Forward | CTATCAAAGAGAAGGCCAA | 16 |
| | Reverse | ATTCTCATAATTTCCTTTCCAT | 17 |
| | Probe | ACAATCTCCTATCCAGAGGTGGTAATC | 18 |
| OPV_17L | Forward | TAATACTTCGATTGCTCATCCAGG | 19 |
| | Reverse | ACTTCTCACAAATGGATTTGAAAATC | 20 |
| | Probe | TCCTTTACGTCATAAATCAT | 21 |
| OPV_F8L | Forward | TCAACTGAAAAGGCCATCTATGA | 22 |
| | Reverse | GAGTATAGAGCACTATTTCTAAATCCCA | 23 |
| | Probe | CCATGCAATATACGTACAAGATAGTAGC | 24 |
| | | CAAC | |

Novel primers and probes described herein target regions of the MPX Clade II genome that are conserved in MPX Clade II, but different enough from analogous regions in MPX Cade I (as well as other orthopoxviruses) to allow definitive detection and identification of MPX Clade II viruses in a sample using a nucleic acid amplification assay. In some embodiments, MPX Clade II can be identified based only on the production of an amplification product; i.e., the primers are specific for MPX Clade II. In other embodiments, MPX Clade II can be identified based on the use of amplification primers in conjunction with a probe, e.g., one that is more specific from MPX Clade II than the primers alone. Exemplary sequences of genomic regions that were determined to be sufficiently conserved in MPX Clade II, but sufficiently different from MPX Clade I (see Example 1), for identification of MPX Clade II viruses in a sample are shown below. In exemplary embodiments, the sequences of genomic regions that were determined to be sufficiently conserved in MPX Clade II, but sufficiently different from MPX Clade I, for identification of MPX Clade II viruses in a sample include the OPG153 gene (see for example, GenBank GeneID: 72551553; Reference Sequence: NC_063383.1), corresponding to Orthopoxvirus A26L/A30L protein. The A26 protein is contained in mature ORPV virions and contributes to the binding of MV to plasma membrane and to the suppression of virus-cell fusion, and in some orthopoxvirus, also is involved in occluding mature virions in dense inclusions. A30L is a small virion phosphoprotein. In certain exemplary embodiments, the sequences of genomic regions that were determined to be sufficiently conserved in MPX Clade II, but sufficiently different from MPX Clade I, for identification of MPX Clade II viruses in a sample include the OPG183 intergenic region (see for example, Gene Accession No. (GenBank) NC_063383.1; GeneID:72551553), corresponding to an intergenic region of orthopoxvirus. In certain exemplary embodiments, the sequences of genomic regions for identification of non-variola orthopoxviruses in a sample includes the F8L gene (similar to Vaccinia virus strain Copenhagen E9L gene: see for example, GenBank Reference Sequence: AF380138.1), corresponding to DNA polymerase, catalytic subunit of MPOX virus strain.

Illustrative primers, probes, and amplicons that can be used in nucleic acid amplification assays to detect MPX Clade II and non-variola orthopoxviruses are shown in Table 2, below. “MPX CII” refers to MPX Clade II (also referred to herein as MPOX Clade II). Forward and reverse primers are designated “Fwd” and “Rev,” respectively. Probes are shown in Table 2 immediately under the reverse primers and are labeled with a fluorescent label and a quencher. Amplicon sequences are shown immediately below the probes.

TABLE 2
Primer, Probe, and Amplicon Nucleotide Sequences for MPOX
Clade II Biomarker Targets

| | | SEQ ID | |
|---|---|---|---|
| Oligo Target/Type | Nucleotide Sequence | NO: | Position |
| MPX CII Fwd (OPG153 | GTTCAAGATCCCTGATTCTAGTCTT | 25 | |
| Gene) | | | |
| MPX CII Rev (OPG153 | GGATCTTGATAGACATCTTAACGAT | 26 | |
| Gene) | | | |
| MPX CII Probe (OPG153 | CTCAGAAGATGCTCCGTTACCGTTT | 27 | |
| Gene) | | | |
| Amplicon (OPG153 | GTTCAAGATCCCTGATTCTAGTCTTTA | 28 | |
| Gene) | GCCTATTTACCTCCTCAGAAGATGCT | | |
| | CCGTTACCGTTTTTACAATCGTTAAG | | |
| | ATGTCTATCAAGATCC | | |
| Ref. Gene Sequence for | NC_063383.1 | 1 | 132396- |
| MPXV OPG153 Gene | | | 136092 |
| MPX CII Fwd (OPG183 | TATACCTATTTCGTCACTTCCACAC | 29 | |
| intergenic region) | | | |
| MPX CII Rev (OPG183 | GGATTACAACATTGATTATGCCTCT | 30 | |
| intergenic region) | | | |
| MPX CII Probe (OPG183 | AGATTCTCCCTCGTCACATGACACC | 31 | |
| intergenic region) | | | |
| Amplicon (OPG183 | TATACCTATTTCGTCACTTCCACACGC | 32 | |
| intergenic region) | TCCGGTAAACGGGTGTCATGTGACG | | |
| | AGGGAGAATCTTGATAAGAGGCATA | | |
| | ATCAATGTTGTAATCC | | |
| Ref. Gene Sequence | NC_063383.1 | 2 | 156355- |
| for MPXV OPG183 | | | 156920 |
| intergenic region | | | |
| MPX Fwd (E9L Gene) | TCAACTGAAAAGGCCATCTATGA | 33 | |
| MPX Rev (E9L Gene) | GAGTATAGAGCACTATTTCTAAATCC | 34 | |
| | CA | | |
| MPX Probe (E9L Gene) | CCATGCAATATACGTACAAGATAGTA | 35 | |
| | GCCAAC | | |
| Amplicon (E9L Gene) | GAGTATAGAGCACTATTTCTAAATCC | 36 | |
| | CATCAGACCATATACTGAGTTGGCTA | | |
| | CTATCTTGTACGTATATTGCATGGAA | | |
| | TCATAGATGGCCTTTTCAGTTGA | | |
| Ref. Gene Sequence for | GenBank AF380138.1 | 3 | 53691 . . . |
| Non-variola E9L Gene | | | 56711 |

Some embodiments of the MPX Clade II biomarker panel assay can detect one or more additional viral targets, such as an MPX Clade I, an MPX virus of either clade (i.e., non-clade-specific MPX detection), or non-variola orthopoxvirus. Primers and probes for this purpose are known and examples are shown in Table 2, above.

Further embodiments of the MPX Clade II biomarker panel assay can detect one or more targets in one or more additional viral pathogens, such as herpes simplex virus (HSV), varicella zoster virus (VZV), syphilis, chlamydia, gonorrhea, Hemophilus ducreyi, or a combination thereof. Infections caused by HSV, VZV, syphilis, chlamydia, Hemophilus ducreyi, and gonorrhea can all cause skin, mouth, and genital lesions. In some embodiments, a genital lesions biomarker panel assay is described herein and can detect MPOX (Clade II and/or Clade I) and one or more other viral pathogens selected from herpes simplex virus (HSV), varicella zoster virus (VZV), syphilis, chlamydia, gonorrhea, Hemophilus ducreyi, or a combination thereof. Primers and probes for this purpose are known and examples are provided in Goldstein, E. J., et al, Diagnostic Microbiology and Infectious Disease, 2021, 99, 2:115221; Cotton, S., et al, Diagnostic Microbiology and Infectious Disease, 2021, 99, 4:115262; and Bennett, S., et al, Journal of Virological Methods, 2013, 189, 1:143-147, which are hereby incorporated by reference for this description.

The considerations for primers and optional probes for detecting MPOX clade II biomarkers are described in more detail below in the section entitled “Exemplary Polynucleotides.”

### Host Response Biomarker Targets

In some embodiments, the detection of MPX Clade II viruses is accompanied by detection of one or more host response biomarkers. As used herein, a host response biomarker refers to a biological compound, such as a polynucleotide or polypeptide which is differentially expressed in a sample taken from a subject having a bacterial infection, a viral infection, or a non-infectious cause of fever as compared to a comparable sample taken from control subjects (e.g., a person with a negative diagnosis, normal or healthy subject, or non-infected subject) or differentially expressed in a sample from a subject having a bacterial infection versus a viral infection. The biomarker can be a nucleic acid, a fragment of a nucleic acid, a polynucleotide, or an oligonucleotide that can be detected and/or quantified. Bacterial infection, viral infection, and non-infectious causes of fever biomarkers include polynucleotides comprising nucleotide sequences from genes or RNA transcripts of genes, including but not limited to, those described in US Patent Publication No. 2022/0298572, which is herein incorporated by reference for this description.

Generally, primers for host biomarker targets are designed to avoid amplification of human genomic DNA (hgDNA) either by having one of the primers span an exon-exon junction or by having the amplicon span an exon-exon junction and relying on a long intron to make PCR amplification of hgDNA unfavorable. In an illustrative design protocol, the genomic sequences of the target nucleic acids were obtained from NCBI (National Center for Biotechnology Information), the locations of the exons and introns were annotated, and cases where there were multiple transcript variants were noted. An exon-exon junction was chosen, ideally with an intron of at least 2 kb in length so that amplification of the genomic sequence would be unlikely when using a short (30-second) PCR extension time. Oligos were designed to minimize primer artifacts whenever possible. General guidelines for primer design generally include the following: Amplicon length of less than 150 bp, Primer length of about 18-25 bp, Primers that span an exon-exon junction and having less than 5 bases on the 3′ end annealing to the second exon, Primer having Tₘ: 60-65° C., Hairpin Tₘs should preferably be no higher than 40-45° C. for forward primer and about 35ºC for reverse primer (less than 30° C. is ideal for both), Self-dimers and hetero-dimers typically shouldn't be more negative than a delta G (kcal/mole) of −9, and Primers should not have self- or hetero-dimers near the 3′ end more negative than a delta G (kcal/mole) of −7.

One, two, three, four, five, or six or more host response biomarkers can be detected by nucleic acid amplification-based methods, such as those described herein. The results of this detection can be used to determine whether a subject has a bacterial infection, a viral infection, both a viral infection and a bacterial infection, or a non-infectious cause of fever. US Patent Publication No. 2022/0298572 describes these biomarkers and methods for making such determinations in detail and is incorporated by reference herein for this description. In particular, US Patent Publication No. 2022/0298572 describes the determination of a bacterial infection score and/or a viral infection score, which are compared to predetermined cutoff values to determine that a subject does, or does not, have a bacterial infection and/or that the subjection does, or does not, have a viral infection (respectively).

The considerations for primers and optional probes for detecting host response biomarkers do not differ from those for detecting MPX Clade II panel biomarkers, such considerations are described in more detail below.

### Controls

In some embodiments, an assay described herein comprises detecting the MPX Clade II biomarkers described above and at least one endogenous control. In some embodiments, the endogenous control is a Sample Adequacy Control (SAC). The SAC ensures that the sample contains human cells or human DNA. This assay includes primers and probes for the detection of a single copy human gene. The SAC signal is only to be considered when the sample is negative for both MPX and OPX analytes. A negative SAC indicates that no human cells are present in the sample due to insufficient mixing of the sample or because of an inadequately collected sample. In some such embodiments, if no MPX Clade II biomarker is detected in a sample, and the SAC is also not detected in the sample, the assay result is considered “invalid” because the sample may have been insufficient. While not intending to be bound by any particular theory, an insufficient sample may be too dilute, contain too little cellular material, or contain an assay inhibitor, etc. In some embodiments, the failure to detect an SAC may indicate that the assay reaction failed. In some embodiments, an endogenous control is an RNA (such as an mRNA, tRNA, ribosomal RNA, etc.). Nonlimiting exemplary endogenous controls include ABL mRNA, GUSB mRNA, GAPDH mRNA, TUBB mRNA, and UPK1a mRNA.

In some embodiments, an assay described herein comprises detecting the MPX Clade II biomarkers described above and at least one exogenous control. In some embodiments, the exogenous control is a Sample Processing Control (SPC). The SPC ensures that the sample is processed correctly. The SPC verifies that sample processing is adequate. Additionally, this control detects sample-associated inhibition of the real-time PCR assay, ensures that the PCR reaction conditions (temperature and time) are appropriate for the amplification reaction, and that the PCR reagents are functional. The SPC should be positive in a negative sample and can be negative or positive in a positive sample. The SPC passes if it meets the validated acceptance criteria. In some such embodiments, if no MPX Clade II biomarker described above is detected in a sample, and the SPC is also not detected in the sample, the assay result is considered “invalid” because there may have been an error in sample processing, including but not limited to, failure of the assay. Nonlimiting exemplary errors in sample processing include, inadequate sample processing, the presence of an assay inhibitor, the presence of a nuclease (such as an RNase), or compromised reagents, etc. In some embodiments, an exogenous control (such as an SPC) is added to a sample. In some embodiments, an exogenous control (such as an SPC) is added during performance of an assay, such as with one or more buffers or reagents. In some embodiments, when a GeneXpert® system is to be used, the SPC is included in the GeneXpert® cartridge. In some embodiments, an exogenous control (such as an SPC) is an Armored RNA®, which is protected by a bacteriophage coat.

In some embodiments, an endogenous control and/or an exogenous control is/are detected contemporaneously, such as in the same assay, as detection of the MPX Clade II biomarkers. In some embodiments, an assay comprises reagents for detecting the MPX Clade II biomarkers described above, and a SAC and/or an exogenous control, simultaneously in the same assay reaction mixture. In some such embodiments, for example, an assay reaction mixture comprises primer sets for amplifying the MPX Clade II biomarkers described above, a primer set for amplifying a SAC and/or a primer set for amplifying an exogenous control, as well as optional labeled probes for detecting the amplification products (such as, for example, TaqMan® probes).

In some embodiments, the assay includes a probe check control (PCC). In some such embodiments, before the start of the PCR reaction, the system (e.g., GeneXpert System) measures the fluorescence signal from the probes to monitor bead rehydration, reaction tube filling, probe integrity, and dye stability. The PCC passes if it meets the validated acceptance criteria.

### Polynucleotides

In some embodiments, polynucleotides are provided for detecting the biomarkers described above. In some embodiments, synthetic polynucleotides are provided. Synthetic polynucleotides, as used herein, refer to polynucleotides that have been synthesized in vitro either chemically or enzymatically. Chemical synthesis of polynucleotides includes, but is not limited to, synthesis using polynucleotide synthesizers, such as OligoPilot™ (GE Healthcare), ABI 3900 DNA Synthesizer (Applied Biosystems), and the like. Enzymatic synthesis includes, but is not limited, to producing polynucleotides by enzymatic amplification, e.g., PCR. A polynucleotide may comprise one or more analog of the canonical nucleotides (e.g., modified nucleotides).

In some embodiments, a polynucleotide is provided that comprises a region that is at least 85%, at least 90%, at least 91%, at least 92%, at least 93%, at least 94%, at least 95%, at least 96%, at least 97%, at least 98%, at least 99%, or 100% identical to, or at least 85%, at least 90%, at least 91%, at least 92%, at least 93%, at least 94%, at least 95%, at least 96%, at least 97%, at least 98%, at least 99%, or 100% complementary to, at least 6, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the MPX Clade II and/or non-variola orthopoxvirus, and/or exemplary controls and other targets discussed above.

In various embodiments, an exemplary polynucleotide comprises at least: 5, 6, 7, 8, 9, 10, 11, 12, 13, 14, 15, 16, 17, 18, 19, 20, 21, 22, 23, 24, 25, 26, 27, 28, 29, or 30 nucleotides. In various embodiments, a polynucleotide comprises fewer than: 200, 150, 100, 50, 40, 30, or 20 nucleotides. In various embodiments, an exemplary polynucleotide is between 6 and 200, between 8 and 200, between 8 and 150, between 8 and 100, between 8 and 75, between 8 and 50, between 8 and 40, between 8 and 30, between 15 and 100, between 15 and 75, between 15 and 50, between 15 and 40, or between 15 and 30 nucleotides long.

In some embodiments, detection of each target nucleic acid can be carried out using a single labeled primer or probe, specific for each target nucleic acid. Different primers and/or probes can have the same label. By using primers or probes labeled with different detectable moieties (e.g., different fluorescent reporter dyes), numerous target nucleic acids can be detected simultaneously in a single reaction mixture. In some embodiments, 2, 3, 4, 5, 6, 7, 8, 9, 10, 11, 12, 13, 14, 15, 16, 17, 18, 19, or 20 or more different labels can be used in a single reaction mixture or a plurality of reaction mixtures. Each target nucleic acid can be independently monitored using such multiplexing technology. In some embodiments, detection of a plurality of target nucleic acids can be carried out using a single labeled primer or probe. A melt curve may be generated in order to distinguish two or more target nucleic acids that each use the same label, but such analysis may not be necessarily required.

### Polynucleotide Modifications

In some embodiments, the methods of detecting at least one target nucleic acid described herein employ one or more polynucleotides that have been modified, such as polynucleotides comprising one or more affinity-enhancing nucleotide analogs. Modified polynucleotides useful in the methods described herein include primers for reverse transcription, PCR amplification primers, and probes. In some embodiments, the incorporation of affinity-enhancing nucleotides increases the binding affinity and specificity of a polynucleotide for its target nucleic acid as compared to polynucleotides that contain only the canonical deoxyribonucleotides, which allows for the use of shorter polynucleotides or for shorter regions of complementarity between the polynucleotide and the target nucleic acid.

In some embodiments, affinity-enhancing nucleotide analogs include nucleotides comprising one or more base modifications, sugar modifications, and/or backbone modifications. In some embodiments, modified bases for use in affinity-enhancing nucleotide analogs include 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine. In some embodiments, affinity-enhancing nucleotide analogs include nucleotides having modified sugars such as 2′-substituted sugars, such as 2′-O-alkyl-ribose sugars, 2′-amino-deoxyribose sugars, 2′-fluoro-deoxyribose sugars, 2′-fluoro-arabinose sugars, and 2′-O-methoxyethyl-ribose (2′MOE) sugars. In some embodiments, modified sugars are arabinose sugars, or d-arabino-hexitol sugars.

In some embodiments, affinity-enhancing nucleotide analogs include backbone modifications such as the use of peptide nucleic acids (PNA: e.g., an oligomer including nucleobases linked together by an amino acid backbone). Other backbone modifications include phosphorothioate linkages, phosphodiester-modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy modifications, and combinations thereof.

In some embodiments, a polynucleotide includes at least one affinity-enhancing nucleotide analog that has a modified base, at least nucleotide (which may be the same nucleotide) that has a modified sugar, and/or at least one internucleotide linkage that is non-naturally occurring.

In some embodiments, an affinity-enhancing nucleotide analog contains a locked nucleic acid (“LNA”) sugar, which is a bicyclic sugar. In some embodiments, a polynucleotide for use in the methods described herein comprises one or more nucleotides having an LNA sugar. In some embodiments, a polynucleotide contains one or more regions consisting of nucleotides with LNA sugars. In other embodiments, a polynucleotide contains nucleotides with LNA sugars interspersed with deoxyribonucleotides. See, e.g., Frieden, M. et al. (2008) Curr. Pharm. Des. 14(11): 1138-1142.

### Primers

In some embodiments, the polynucleotide is a primer. Primers useful in the methods described herein are generally capable of selectively hybridizing to: genomic DNA, a target RNA (genomic or transcript), a cDNA reverse transcribed from the target RNA, and/or an amplicon that has been amplified from genomic DNA, a target RNA, or a cDNA (collectively referred to as “template”), and, in the presence of the template, a polymerase and suitable buffers and reagents, can be extended to form a primer extension product. Primers are generally of a sufficient length to ensure selective hybridization to their target nucleic acids. Generally, primers of at least 15 nucleotides in length hybridize specifically in most contexts, and this length can be reduced, e.g., by including of affinity-enhancing modifications, such as those discussed above. Primers can but need not be exactly complementary to their target nucleic acids. Primers can have any degree of complementarity described above for exemplary polynucleotides. In illustrative embodiments, primers can be 8 to 40 nucleotides in length and at least 90% complementary to their target nucleic acids: 8 to 40 nucleotides in length and at least 95% complementary to their target nucleic acids: 8 to 40 nucleotides in length and at least 99% complementary to their target nucleic acids: 8 to 30 nucleotides in length and at least 90% complementary to their target nucleic acids: 8 to 30 nucleotides in length and at least 95% complementary to their target nucleic acids: 8 to 30 nucleotides in length and at least 99% complementary to their target nucleic acids. In embodiments wherein a primer is less than 100% complementary to it target nucleic acid, having the 3′ nucleotide in the primer be complementary to its target nucleic acid facilitates the production of an extension product.

In some embodiments, a primer that selectively hybridizes to its target nucleic acid hybridizes to its target nucleic acid with at least 5-fold greater affinity than to non-target nucleic acid under the same assay conditions. In some embodiments, a primer that selectively hybridizes to its target nucleic acid hybridizes to its target nucleic acid with at least 10-fold greater affinity than to non-target nucleic acid under the same assay conditions.

In some embodiments, a primer pair is designed to produce an amplicon that is 50 to 1500 nucleotides long, 50 to 1000 nucleotides long, 50 to 750 nucleotides long, 50 to 500 nucleotides long, 50 to 400 nucleotides long, 50 to 300 nucleotides long, 50 to 200 nucleotides long, 50 to 150 nucleotides long, 100 to 300 nucleotides long, 100 to 200 nucleotides long, or 100 to 150 nucleotides long.

In some embodiments, the primer is labeled with a detectable moiety. In some embodiments, a primer is not labeled.

### Probes

In some embodiments, the polynucleotide is a probe. Probes useful in the methods described herein are generally capable of selectively hybridizing to: genomic DNA, a target RNA (genomic or transcript), a cDNA reverse transcribed from the target RNA, and/or an amplicon that has been amplified from genomic DNA, a target RNA, or a cDNA (collectively referred to as “template”). Generally, probes of at least 15 nucleotides in length hybridize specifically in most contexts, and this length can be reduced, e.g., by including of affinity-enhancing modifications, such as those discussed above. Probes can but need not be exactly complementary to their target nucleic acids. Probes can have any degree of complementarity described above for exemplary polynucleotides. In illustrative embodiments, probes can be 8 to 40 nucleotides in length and at least 90% complementary to their target nucleic acids: 8 to 40 nucleotides in length and at least 95% complementary to their target nucleic acids: 8 to 40 nucleotides in length and at least 99% complementary to their target nucleic acids: 8 to 30 nucleotides in length and at least 90% complementary to their target nucleic acids: 8 to 30 nucleotides in length and at least 95% complementary to their target nucleic acids: 8 to 30 nucleotides in length and at least 99% complementary to their target nucleic acids. In embodiments wherein a primer is less than 100% complementary to a target nucleic acid, any points or regions of non-complementarity are typically located so as not to disrupt the ability of the probe to selectively hybridize to its target nucleic acid.

In some embodiments, a probe that selectively hybridizes to its target nucleic acid hybridizes to its target nucleic acid with at least 5-fold greater affinity than to non-target nucleic acid under the same assay conditions. In some embodiments, a probe that selectively hybridizes to its target nucleic acid hybridizes to its target nucleic acid with at least 10-fold greater affinity than to non-target nucleic acid under the same assay conditions.

### Polynucleotide Labels

In some embodiments, the primer or probe is labeled with a detectable moiety. Detectable moieties include directly detectable moieties, such as fluorescent dyes, and indirectly detectable moieties, such as members of binding pairs. When the detectable moiety is a member of a binding pair, in some embodiments, the probe can be detectable by incubating the probe with a detectable label bound to the second member of the binding pair. In some embodiments, a primer or probe is not labeled, such as when a primer or probe is immobilized, e.g., on a microarray or bead. A labeled primer is extendable, e.g., by a polymerase. In some embodiments, a probe is extendable. In other embodiments, a probe is not extendable. The following discussion centers on probes, as these are more typically employed for detecting in the methods described here, but those of skill in the art appreciate that the polynucleotide labeling strategies described below apply equally to the labeling of primers.

In some embodiments, the probe is a FRET probe that, in some embodiments, is labeled at the 5′-end with a fluorescent dye (donor) and at the 3′-end with a quencher (acceptor), a chemical group that absorbs (i.e., suppresses) fluorescence emission from the dye when the groups are in close proximity (e.g., attached to the same probe). Thus, in some embodiments, the emission spectrum of the dye should overlap considerably with the absorption spectrum of the quencher. In other embodiments, the dye and quencher are not at the ends of the FRET probe.

Illustrative FRET probes, which include, but are not limited to, a TaqMan′R probe, a Molecular beacon probe and a Scorpion probe. A TaqMan′R; probe is a linear probe that typically has a fluorescent dye covalently bound at one end of the DNA and a quencher molecule covalently bound elsewhere, such as at the other end of the DNA. The FRET probe comprises a sequence that is complementary to a region of the cDNA or amplicon such that, when the FRET probe is hybridized to the cDNA or amplicon, the dye fluorescence is quenched, and when the probe is digested during amplification of the cDNA or amplicon, the dye is released from the probe and produces a fluorescence signal. In some embodiments, the amount of target nucleic in the sample is proportional to the amount of fluorescence measured during amplification.

Like TaqMan′R probes, Molecular Beacons use FRET to detect a PCR product via a probe having a fluorescent dye and a quencher attached at the ends of the probe. Unlike TaqMan® probes, Molecular Beacons remain intact during the PCR cycles. Molecular Beacon probes form a stem-loop structure when free in solution, thereby allowing the dye and quencher to be in close enough proximity to cause fluorescence quenching. When the Molecular Beacon hybridizes to a target nucleic acid, the stem-loop structure is abolished so that the dye and the quencher become separated in space and the dye fluoresces. Molecular Beacons are available, e.g., from Gene Link™ (see www.genelink.com/newsite/products/mbintro.asp).

In some embodiments, Scorpion probes can be used as sequence-specific primers and for PCR product detection. Like Molecular Beacons, Scorpion probes form a stem-loop structure when not hybridized to a target nucleic acid. However, unlike Molecular Beacons, a Scorpion probe achieves both sequence-specific priming and PCR product detection. A fluorescent dye molecule is attached to the 5′-end of the Scorpion probe, and a quencher is attached elsewhere, such as to the 3′-end. The 3′ portion of the probe is complementary to the extension product of the PCR primer, and this complementary portion is linked to the 5′-end of the probe by a non-amplifiable moiety. After the Scorpion primer is extended, the target-specific sequence of the probe binds to its complement within the extended amplicon, thus opening up the stem-loop structure and allowing the dye on the 5′-end to fluoresce and generate a signal. Scorpion probes are available from, e.g., Premier Biosoft International (see www.premierbiosoft.com/tech_notes/Scorpion.html).

In some embodiments, labels that can be used on the FRET probes include colorimetric and fluorescent dyes, such as Alexa Fluor dyes: BODIPY dyes, such as BODIPY FL, Cascade Blue, and Cascade Yellow: coumarin and its derivatives, such as 7-amino-4-methylcoumarin, aminocoumarin and hydroxycoumarin: cyanine dyes, such as Cy3 and Cy5: eosins and erythrosins: fluorescein and its derivatives, such as fluorescein isothiocyanate: macrocyclic chelates of lanthanide ions, such as Quantum Dye™: Marina Blue: Oregon Green: rhodamine dyes, such as rhodamine red, tetramethylrhodamine and rhodamine 6G: Texas Red: fluorescent energy transfer dyes, such as thiazole orange-ethidium heterodimer; and TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR: Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′, 4′,5 ′,7′-Tetrabromosulfonefluorescein, and TET.

Examples of dye/quencher pairs (i.e., donor/acceptor pairs) include, but are not limited to, fluorescein/tetramethylrhodamine: IAEDANS/fluorescein: EDANS/dabcyl: fluorescein/fluorescein: BODIPY FL/BODIPY FL; and fluorescein/QSY 7 or QSY 9 dyes. When the donor and acceptor are the same, FRET may be detected, in some embodiments, by fluorescence depolarization. Certain specific examples of dye/quencher pairs (i.e., donor/acceptor pairs) include, but are not limited to, Alexa Fluor 350/Alexa Fluor 488: Alexa Fluor 488/Alexa Fluor 546: Alexa Fluor 488/Alexa Fluor 555: Alexa Fluor 488/Alexa Fluor 568: Alexa Fluor 488/Alexa Fluor 594: Alexa Fluor 488/Alexa Fluor 647: Alexa Fluor 546/Alexa Fluor 568: Alexa Fluor 546/Alexa Fluor 594: Alexa Fluor 546/Alexa Fluor 647: Alexa Fluor 555/Alexa Fluor 594: Alexa Fluor 555/Alexa Fluor 647: Alexa Fluor 568/Alexa Fluor 647: Alexa Fluor 594/Alexa Fluor 647: Alexa Fluor 350/QSY35: Alexa Fluor 350/dabcyl: Alexa Fluor 488/QSY 35: Alexa Fluor 488/dabcyl: Alexa Fluor 488/QSY 7 or QSY 9; Alexa Fluor 555/QSY 7 or QSY9: Alexa Fluor 568/QSY 7 or QSY 9: Alexa Fluor 568/QSY 21: Alexa Fluor 594/QSY 21; and Alexa Fluor 647/QSY 21. In some instances, the same quencher may be used for multiple dyes, for example, a broad spectrum quencher, such as an Iowa Black® quencher (Integrated DNA Technologies, Coralville, IA) or a Black Hole Quencher™ (BHQ™: Sigma-Aldrich, St. Louis, MO).

Specific examples of fluorescently labeled ribonucleotides useful in the preparation of probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences (GE Healthcare), such as Cy3-UTP and Cy5-UTP.

Specific examples of fluorescently labeled deoxyribonucleotides useful in the preparation of probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP: Alexa Fluor 488-7-OBEA-dCTP, Alexa Fluor 546-16-OBEA-dCTP, Alexa Fluor 594-7-OBEA-dCTP, and Alexa Fluor 647-12-OBEA-dCTP. Fluorescently labeled nucleotides are commercially available and can be purchased from, e.g., Invitrogen.

As noted above, exemplary detectable moieties also include members of binding pairs. Exemplary binding pairs include, but are not limited to, biotin and streptavidin, antibodies and antigens, etc.

### Sample

The sample to be tested can be any sample suspected of containing at least the MPX Clade II biomarkers described herein. In some embodiments, the sample is a biological sample collected from a subject. In other embodiments, the sample is a sample that is not collected directly from a subject, such as, e.g., a wastewater sample or a sample from an air filter in a building.

Illustrative biological samples include skin samples, lesion swabs, vesicular lesion fluid samples, pustular lesion fluid samples, rectal samples, and samples of bodily fluids, such as nasal aspirates, nasal washes, nasal swabs, nasopharyngeal swabs, saliva, oropharyngeal swabs, throat swabs, bronchoalveolar lavage samples, bronchial aspirates, bronchial washes, endotracheal aspirates, endotracheal washes, tracheal aspirates, nasal secretion samples, mucus samples, sputum samples, plasma samples, whole blood samples, etc.

The sample to be tested is, in some embodiments, fresh (i.e., never frozen). In other embodiments, the sample is a frozen specimen. In some embodiments, the sample is a tissue sample, such as a formalin-fixed paraffin embedded sample. In some embodiments, the sample is a liquid cytology sample.

In some embodiments, a sample to be tested is contacted with a buffer after collection. For example, in the case of skin sample, lesion swab, vesicular lesion fluid sample, pustular lesion fluid sample, or rectal samples, a buffer (including, e.g., a preservative) can be added to the sample. In embodiments where the sample is a swab sample, the swab can simply be placed in a buffer. In some embodiments, that sample is contacted with the buffer immediately: in the case of a swab, the swab is immediately placed in the buffer. In some embodiments, the sample (e.g., including the swab) is contacted with buffer within 5 minutes, within 10 minutes, within 30 minutes, within 1 hour, or within 2 hours of sample collection.

In some embodiments, less than 5 ml, less than 4 ml, less than 3 ml, less than 2 ml, less than 1 ml, or less than 0.75 ml of sample or buffered sample are used in the present methods. In some embodiments, 0.1 ml to 1 ml of sample or buffered sample is used in the present methods.

### Subjects

A biological sample useful in the methods described herein can be collected from any subject that can be infected by one, several, or all of the viruses described above. In various embodiments, the subject can include non-human animals, e.g., canines, felines, equines, primates, and other non-human mammals, as well as humans.

In some embodiments, the sample to be tested is obtained from an individual who has one or more symptoms of orthopoxvirus, e.g., MPX, infection or who has been exposed to such an individual. The primary symptoms of MPOX virus are fever, headache, myalgia, lymphadenopathy, and fatigue before the appearance of vesiculopustular rash lasting approximately 2-3 weeks. In some such embodiments, the individual is monitored for recurrence of orthopoxvirus, e.g., MPX.

In some embodiments, methods described herein can be used for routine screening of apparently healthy individuals with no risk factors. In some embodiments, methods described herein are used to screen asymptomatic individuals, for example, during routine or preventative care. In some embodiments, methods described herein are used to screen women who are pregnant or who are attempting to become pregnant.

In some embodiments, the methods described herein can be used to assess the effectiveness of a treatment in an individual undergoing treatment a condition caused by an orthopoxvirus (e.g., MPX).

### Assay Methods

Any analytical procedure capable of permitting specific detection of a target nucleic acid can be used in the methods herein presented. In some embodiments, DNA targets can be detected by direct hybridization or, more easily, by amplification of the DNA template and detection of the amplicon. In some embodiments, RNA targets can be detected by direct hybridization or, more easily, by reverse transcribing a target RNA to produce a cDNA that is complementary to the target RNA. This cDNA can be directly detected by direct hybridization or by amplification of the cDNA template.

Nucleic acid amplification provides rapid, sensitive, and specific detection of nucleic acid targets, and has been employed in a wide variety of assay formats to detect nucleic acid targets. Those of skill in the art can, following the guidance herein, carry out the methods described herein in any number of different nucleic acid amplification-based assays, using, for example, any of the nucleic acid amplification methods discussed above. Such methods can entail thermocycling, but need not do so, as in the case of isothermal amplification. Exemplary methods include, but are not limited to, isothermal amplification, real time RT-PCR, endpoint RT-PCR, and amplification using T7 polymerase from a T7 promoter annealed to a DNA, such as provided by the SenseAmp Plus™ Kit available at Implen, Germany. Amplification and detection can be carried out in solution or can make use of a solid support (e.g., a biochip). Nucleic acid amplification-based assays can employ a single reaction chamber or multiple reaction chambers. Amplification can be nested or non-nested. In some embodiments, detection includes electrochemical detection.

In some embodiments, target nucleic acids, such as MPX Clade II biomarkers (as well as Clade I and other orthopoxvirus biomarkers), optional host biomarkers, and/or optional controls, can be detected by (a) contacting nucleic acid from the sample with a set of primers and optional probes for detecting the presence of the desired target nucleic acids. (b) subjecting the nucleic acid, primers, and optional probes to amplification conditions: (c) detecting the presence of any amplification product(s), optionally via real-time PCR, melt curve analysis, or a combination thereof, and (d) differentially identifying the presence of a viral pathogen in the sample, or determining that no viral pathogen detectable using the set of primers is present, based on detection of the amplification product(s) or lack thereof, respectively. In this context, “differentially identifying” refers to the ability to determine that a particular target organism is present and that one or more other target organisms of the assay are not. In some embodiments, the assay is able to determine the presence of any target organism this present in the sample, while ruling out the presence of the other target organisms (above the detection limit of the assay).

In some embodiments of amplification by polymerase chain reaction (PCR), an exemplary cycle comprises an initial denaturation at 90° ° C. to 100° ° C. for 20 seconds to 5 minutes, followed by cycling that comprises denaturation at 90° ° C. to 100° ° C. for 1 to 10 seconds, followed by annealing and amplification at 60° ° C. to 75° C. for 10 to 40 seconds. A further exemplary cycle comprises 20 seconds at 94° C., followed by up to 3 cycles of 1 second at 95° C., 35 seconds at 62° C., 20 cycles of 1 second at 95° C., 20 seconds at 62° C., and 14 cycles of 1 second at 95° C., 35 seconds at 62° C. In some embodiments, for the first cycle following the initial denaturation step, the cycle denaturation step is omitted. In some embodiments, Taq polymerase is used for amplification. In some embodiments, the cycle is carried out at least 10 times, at least 15 times, at least 20 times, at least 25 times, at least 30 times, at least 35 times, at least 40 times, or at least 45 times. In some embodiments, Taq is used with a hot-start function. In some embodiments, detection of the target nucleic acids occurs in less than 3 hours, less than 2.5 hours, less than 2 hours, less than 1 hour, or less than 30 minutes from initial denaturation through the last extension. In some embodiments, target nucleic acids are detected by a method that includes real-time quantitative PCR, e.g., using FRET probes, such as those described above.

In some embodiments, quantitation of the results of real-time PCR assays is done by constructing a standard curve from a nucleic acid of known concentration and then extrapolating quantitative information for target nucleic acids of unknown concentration. In some embodiments, the nucleic acid used for generating a standard curve is a DNA (for example, an endogenous control, or an exogenous control). In some embodiments, the nucleic acid used for generating a standard curve is a purified double-stranded plasmid DNA or a single-stranded DNA generated in vitro.

In some embodiments, in order for an assay to indicate that a given target nucleic acid is not present in a sample, the Ct values for an endogenous control (such as an SAC) and/or an exogenous control (such as an SPC) must be within previously-determined valid ranges. For example, in some embodiments, the absence of a particular target nucleic acid cannot be confirmed unless the controls are detected, indicating that the assay was successful.

In some embodiments, a threshold Ct (or a “cutoff Ct”) value for a target nucleic acid (including an endogenous control and/or exogenous control), below which the gene is considered to be detected, has previously been determined. In some embodiments, a threshold Ct is determined using substantially the same assay conditions and system (such as a GeneXpert®) on which the samples will be tested.

Real-time PCR is performed using any PCR instrumentation available in the art. Typically, instrumentation used in real-time PCR data collection and analysis comprises a thermal cycler, optics for fluorescence excitation and emission collection, and optionally a computer and data acquisition and analysis software.

In some embodiments, the number of target nucleic acids in an assay exceeds the number of labels that can be detected, e.g., in a particular instrument. Therefore, the PCR amplification can be followed by a melt analysis to increase the number of possible reported results. In general, target organisms requiring high sensitivity, e.g., viruses, can be detected with real-time PCR detection using TaqMan probes or molecular beacon probes and/or detected with melt analysis.

Another approach to detect target nucleic acids can include high-resolution melt alone. For example, a nested multiplex PCR can be conducted by first performing reverse transcription, followed by a multiplexed first-stage PCR reaction (PCR1). Multiple simultaneous second-stage PCR reactions (PCR2) are then performed in an array to amplify sequences within the PCR1 products. Endpoint melting curve data to detect target nucleic acids and analyses can then be performed to generate a result for each analyte.

Target nucleic acids can also be detected by real-time PCR but in more than one reaction chambers. Another approach to detect a target nucleic acid can include digital microfluidics or electrowetting and electrochemical detection. For example, digital microfluidics or electrowetting, responsible for the movement and transfer of samples and reagents inside a cartridge can be conducted. Systems for such can include a microarray for detection, consisting of target-specific capture probes attached to gold electrodes (solid-support), which generates a voltage signal if a “target DNA/signal probe” hybridizes with the capture probes. Target nucleic acids can also be detected using a chip that includes an integrated sensor array.

Examples of other approaches that can be employed in the methods describe herein include bead-based flow cytometric assay. See Lu J. et al. (2005) Nature 435:834-838, which is incorporated herein by reference for this description. An example of a bead-based flow cytometric assay is the xMAP® technology of Luminex, Inc. See www.luminexcorp.com/technology/index.html. Another approach uses microfluidic devices and single-molecule detection. See U.S. Pat. Nos. 7,402,422 and 7,351,538 to Fuchs et al, U.S. Genomics, Inc., each of which is incorporated herein by reference in its entirety. Yet another approach is simple gel electrophoresis and detection with labeled probes (e.g., probes labeled with a radioactive or chemiluminescent label), such as by northern blotting.

In some embodiments, the approach for detecting a target nucleic acid does not include bead-based flow cytometric assay, microfluidic devices and single-molecule detection, simple gel electrophoresis, use of a capture probe attached to a solid-support, separation of reaction mixture into multiple reaction chambers, array-based detection, nested amplification, electrochemical detection, high resolution melt only, or a combination thereof.

### Automated Assay Methods

Readily automated approaches are of great interest. The methods described herein can be carried out in a substantially automated manner using a commercially available nucleic acid amplification system. Exemplary nonlimiting nucleic acid amplification systems that can be used to carry out the methods of the invention include the GENEXPERTR system, a GENEXPERTR Infinity system, and GENEXPERTR Xpress System (Cepheid, Sunnyvale, Calif.). In some embodiments, the amplification system may be available at the same location as the individual to be tested, such as a health care provider's office, a clinic, or a community hospital, so processing is not delayed by transporting the sample to another facility. Assays according to the method described herein can be completed in under 3 hours, in some embodiments, under 2 hours, in some embodiments, under 1 hour, in some embodiments, under 45 minutes, in some embodiments, under 35 minutes, and in some embodiments, under 30 minutes, using an automated system, for example, the GENEXPERT® system. The GENEXPERTR utilizes a self-contained, single-use cartridge. Sample extraction, amplification, and detection may all carried out within this self-contained sample cartridge as described herein.

In some embodiments, after the sample is added to the cartridge, the sample is contacted with lysis buffer and released nucleic acid (NA) is bound to a NA-binding substrate, such as a silica or glass substrate. The sample supernatant is then removed and the NA eluted in an elution buffer, such as a Tris/EDTA buffer. The eluate may then be processed in the cartridge to detect target nucleic acids as described herein. In some embodiments, the eluate is used to reconstitute at least some of the PCR reagents, which are present in the cartridge as lyophilized particles.

A cartridge having a plurality of chambers can have the set of primers and optional probes described herein, or a subset thereof, disposed in a chamber. In some embodiments, the set of primers and optional probes described herein, or a subset thereof, are disposed in more than one of the plurality of chambers.

In some embodiments, RT-PCR is used to amplify and analyze the presence of the target nucleic acids. In some embodiments, the reverse transcription uses MMLV and/or CAT-A RT enzyme and an incubation of 5 to 20 minutes at 40° C. to 50° C. In some embodiments, the PCR uses Taq polymerase with hot-start function, such as AptaTaq (Roche). In some embodiments, the initial denaturation is at 90° ° C. to 100° ° C. for 20 seconds to 5 minutes: the cycling denaturation temperature is 90° ° C. to 100° C. for 1 to 10 seconds; the cycling anneal and amplification temperature is 60° ° C. to 75° C. for 10 to 40 seconds; and up to 50 cycles are performed.

In some embodiments, a double-denature method is used to amplify low-copy number target nucleic acids. A double-denature method comprises, in some embodiments, a first denaturation step followed by addition of primers and/or probes for detecting target nucleic acids. All or a substantial portion of the nucleic acid-containing sample (such as a DNA eluate) is then denatured a second time before, in some instances, a portion of the sample is aliquotted for cycling and detection of the target nucleic acids. While not intending to be bound by any particular theory, the double-denature protocol may increase the chances that a low-copy number target nucleic acid (or its complement) will be present in the aliquot selected for cycling and detection because the second denaturation effectively doubles the number of target nucleic acids (i.e., it separates the target nucleic acid and its complement into two separate templates) before an aliquot is selected for cycling. In some embodiments, the first denaturation step comprises heating to a temperature of 90° ° C. to 100° C. for a total time of 30 seconds to 5 minutes. In some embodiments, the second denaturation step comprises heating to a temperature of 90° ° C. to 100° C. for a total time of 5 seconds to 3 minutes. In some embodiments, the first denaturation step and/or the second denaturation step is carried out by heating aliquots of the sample separately. In some embodiments, each aliquot may be heated for the times listed above. As a non-limiting example, a first denaturation step for an NA-containing sample (such as a DNA eluate) may comprise heating at least one, at least two, at least three, or at least four aliquots of the sample separately (either sequentially or simultaneously) to a temperature of 90° ° C. to 100° C. for 60 seconds each. As a non-limiting example, a second denaturation step for a NA-containing sample (such as a DNA eluate) containing enzyme, primers, and probes may comprise heating at least one, at least two, at least three, or at least four aliquots of the eluate separately (either sequentially or simultaneously) to a temperature of 90° ° C. to 100° C. for 5 seconds each. In some embodiments, an aliquot is the entire NA-containing sample (such as a DNA eluate). In some embodiments, an aliquot is less than the entire NA-containing sample (such as a DNA eluate).

In some embodiments, an off-line centrifugation is used, for example, with samples with low cellular content. The sample, with or without a buffer added, is centrifuged and the supernatant removed. The pellet is then resuspended in a smaller volume of either supernatant or the buffer. The resuspended pellet is then analyzed as described herein.

### Exemplary Automation and Systems

Many existing fully integrated nucleic acid amplification and test systems capable of sample preparation are normally quite complicated and costly. The nucleic acid amplification and test systems provided herein perform rapid, simple, convenient, and affordable nucleic acid analysis.

### System Overview

In one aspect, the invention pertains to a sample cartridge that utilizes a valve body platform that allows for detection of enveloped and free target nucleic acids. In some embodiments, the valve body includes a sample processing region or lysing chamber that provides for either or both mechanical and chemical lysis. This allows a single cartridge to provide lysing for a multitude of differing types of targets, thus, can be considered an “panel assay cartridge.” In some embodiments, the sample cartridge can perform processing and detection of viral targets suited for chemical lysing.

The sample cartridge device can be any device configured to perform one or more process steps relating to preparation and/or analysis of a biological fluid sample according to any of the methods described herein. In some embodiments, the sample cartridge device is configured to perform at least sample preparation. The sample cartridge can further be configured to perform additional processes, such as detection of a target nucleic acid in a nucleic acid amplification test (NAAT), e.g., Polymerase Chain Reaction (PCR) assay, by use of a reaction vessel attached to the sample cartridge. In some embodiments, the reaction vessel extends from the body of the cartridge. Preparation of a fluid sample generally involves a series of processing steps, which can include chemical, electrical, mechanical, thermal, optical or acoustical processing steps according to a specific protocol. Such steps can be used to perform various sample preparation functions, such as cell capture, cell lysis, binding of analyte, and binding of unwanted material.

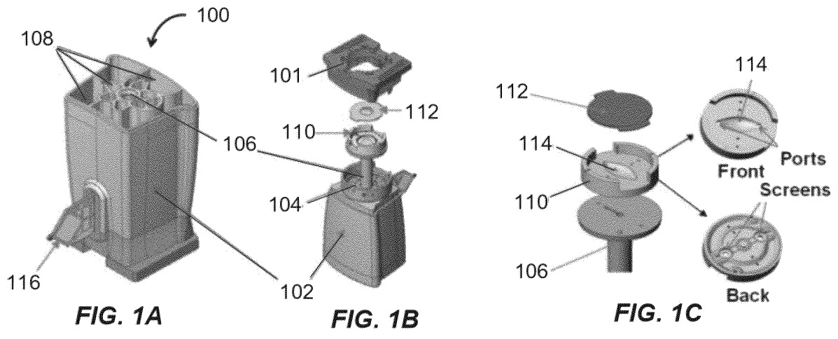

A sample cartridge suitable for use with the invention, includes one or more transfer ports through which the prepared fluid sample can be transported into an attached reaction vessel for analysis. FIG. 1A illustrates an exemplary panel assay cartridge 100 suitable for sample preparation and analytics testing by PCR when received in an instrument module in accordance with some embodiments. The sample cartridge is attached with a reaction vessel 116 (also referred to as a “reaction tube” or “PCR tube”) adapted for analysis of a fluid sample processed within the sample cartridge 100. In some embodiments the reaction vessel extends from the cartridge body. Such a sample cartridge 100 includes various components including a main housing 102 having one or more chambers 108 for processing of the fluid sample, which typically include sample preparation before analysis. In these embodiments, the sample cartridge can be a fully integrated nucleic acid amplification and test system combining sample preparation, amplification, and detection together. The instrument module facilitates the processing steps needed to perform sample preparation and the prepared sample is transported through one of a pair of transfer ports into fluid conduit of the reaction vessel 116 attached to the housing of the sample cartridge 100. The prepared biological fluid sample is then transported into a reaction chamber of the reaction vessel where the biological fluid sample undergoes nucleic acid amplification. In some embodiments, the amplification is a polymerase chain reaction. In some embodiments, concurrent with the amplification of the biological fluid sample, an excitation means, and an optical detection means of the module is used to detect optical emissions that indicate the presence or absence of a target nucleic acid analyte of interest, e.g., a bacterium, a virus, a pathogen, a toxin, or other target analyte. It is appreciated that such a reaction vessel could include various differing chambers, conduits, or micro-well arrays for use in detecting the target analyte. The sample cartridge can be provided with means to perform preparation of the biological fluid sample before transport into the reaction vessel. Any chemical reagent required for viral or cell lysis or means for binding or detecting an analyte of interest (e.g., reagent beads) can be contained within one or more chambers of the sample cartridge, and as such can be used for sample preparation.

An exemplary use of a reaction vessel for analyzing a biological fluid sample is described in commonly assigned U.S. Pat. No. 6,818,185, entitled “Cartridge for Conducting a Chemical Reaction,” filed May 30, 2000, the entire contents of which are incorporated herein by reference for all purposes. Examples of the sample cartridge and associated modules are shown and described in U.S. Pat. No. 6,374,684, entitled “Fluid Control and Processing System” filed Aug. 25, 2000, and U.S. Pat. No. 8,048,386, entitled “Fluid Processing and Control,” filed Feb. 25, 2002, U.S. Patent Application No. 63/217,672 entitled “Universal Assay Cartridge and Methods of Use” filed Jul. 1, 2021: U.S. Provisional Application No. 63/319,993 entitled “Unitary Cartridge Body and Associated Components and Methods of Manufacture” filed Mar. 15, 2022; and U.S. Pat. No. 10,562,030 entitled “Molecular Diagnostic Assay System” filed Jul. 22, 2016: the entire contents of which are incorporated herein by reference in their entirety for all purposes. The above noted patents are included in the attached appendix.

Various aspects of the sample cartridge 100 can be further understood by referring to U.S. Pat. No. 6,374,684 “the '684 patent”), which described certain aspects of a sample cartridge in greater detail. Such sample cartridges can include a fluid control mechanism, such as a rotary fluid control valve assembly, that is fluidically connected to the chambers of the sample cartridge. The term “chamber” can be used interchangeably with the terms “well”, “tube”, and the like. Rotation of the rotary fluid control valve permits fluidic communication between chambers and the valve so as to control flow of a biological fluid sample deposited in the cartridge into different chambers in which various reagents can be provided according to a particular protocol as needed to prepare the biological fluid sample for analysis. To operate the rotary valve, the cartridge processing module comprises a motor such as a stepper motor that is typically coupled to a drive train that engages with a feature of the valve in the sample cartridge to control movement of the valve in coordination with movement of the syringe, thereby resulting movement of the fluid sample according to the desired sample preparation protocol. The fluid metering and distribution function of the rotary valve according to a particular sample preparation protocol is demonstrated in the '684 patent.

### Exemplary Assay Cartridge and Valve Assemblies

### Overview

As shown in FIG. 1A, the panel assay cartridge 100 comprises a cartridge body 102 containing a plurality of chambers 108 for reagents or buffers and sample processing. The chambers are disposed around a central syringe barrel 106 that is in fluid communication with a valve body 110 (see FIGS. 1B and 1C) and that is sealed with a gasket 104. The valve body 110 can include a cap 112 and the entire cartridge body can be supported on a cartridge base 101. The valve body typically contains one or channels or cavities (chamber(s) 114) that can contain a filter as described herein that can function to bind and elute a nucleic acid. In some embodiments the cartridge further comprises one or more temperature-controlled channels or chambers that can, in certain embodiments, function as thermocycling chambers. A “plunger” not shown can be operated to draw fluid into the syringe barrel 106 and rotation of the valve body 110 provides selective fluid communication between the various reagent chambers 108 and channels, reaction chamber(s), mixing chambers, and optionally, any temperature controlled regions. Thus, the various reagent chambers 108, reaction chambers, filter material(s), and temperature-controlled chambers or channels are selectively in fluid communication by rotation of the plunger and reagent movement (e.g., chamber loading or unloading) is operated by the “syringe” action of the plunger within the valve assembly. In other embodiments, the various reagent chambers, reaction chambers, filter material, and temperature-controlled chambers or channels are selectively in fluid communication by linear progression (e.g., by forced movement) of the reagents and sample from one chamber to the next.

### Exemplary Valve Assemblies

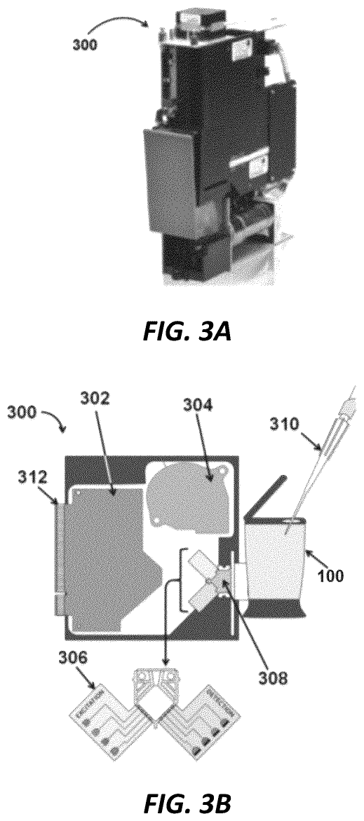

FIG. 3 illustrates differing valve assembles that can be used in the sample cartridge of FIG. 1A. Each of the valve assemblies is shown inverted to better illustrate the components of the valve body. Valve assembly A performs only mechanical lysing and is suitable for lysing hardy targets (e.g., certain bacteria, spores). Valve assemblies B and C perform only chemical lysing and is suitable for lysing less hardy targets (e.g. viruses, free NA, some spores, some bacteria and yeasts). Valve assembly D can perform both mechanical and chemical lysing for all types of targets. In all such cartridges, the valve assembly includes the syringe tube 106, valve body 110, and valve cap 112. In one aspect, the additional capabilities of the valve assembly of the sample cartridge rely in part on the filter, features of the valve body and cap, as well as the particular workflow sequence performed by the instrument interface of the module. For example, valve assembly A has a valve body 110 shaped with a circular cavity to support a polymer filter disc 116 to filter the sample, and the cap 112 has a sonication dome feature 113, which interfaces with a sonication horn of a cartridge receiving module so as to ultrasonically lyse the target. By contrast, valve assemblies B and C have a valve body 110 with an oblong filter recess 111 that receives a glass filter 114 (e.g. glass column) therein, the glass filter configured for binding with nucleic acid released from the target by chemical lysing. Valve assembly D has a design more similar to valve assembly A, having a cap 112 with a sonication dome 113, and a valve body with a circular cavity for supporting a disc filter, however this design uses a glass fiber filter 117. Utilizing glass fibers to form the filter facilitates affinity bonding with the free nucleic acid released by chemical lysing. Thus, this filter is suited for both mechanical and chemical lysing. U.S. Publication No. 2023/0044516 further details the assay cartridge of valve assembly D and is incorporated herein by reference in its entirety.

### Reaction Modules

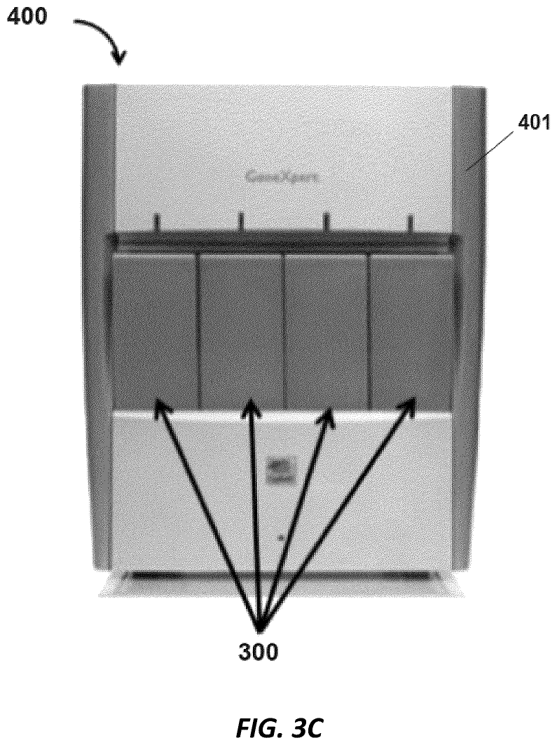

In certain embodiments the cartridge 200 is configured for insertion into a reaction module 300, e.g., as shown in FIG. 4A. As illustrated in FIG. 4B the module is configured to receive the cartridge 200 therein. In certain embodiments the reaction module provides heating plates 308 to heat the temperature controlled chamber or channel. The module can optionally additionally include a fan 304 to provide cooling where the temperature controlled channel or chamber is a thermocycling channel or chamber. Electronic circuitry 302 can be provided to pass information (e.g., optical information) top a computer for analysis. In certain embodiments the module can contain optical blocks 306 to provide excitation and/or detection of one or more (e.g., 1, 2, 3, 4, or more) optical signals representing, e.g., signal DNAs amplified for various PCR targets. In various embodiments an electrical connector 312 can be provided for interfacing the module with a system (e.g. system controller or with a discrete analysis/controller unit. As illustrated, in FIG. 4B the sample can be introduced into the cartridge using a pipette 310. In certain embodiments, the module also contains a controller that operates a plunger in the syringe barrel and the rotation of the valve body.

### Analytical System

In certain embodiments a system (e.g., a processing unit) is provided. One illustrative, but non-limiting embodiment is shown in FIG. 4C. System 400 includes an enclosure 401 that is configured to support and power multiple sample processing modules 300, where each processing module is configured to hold and operate a removable cartridge 100. In some embodiments, the system is configured to operate the sample processing modules to perform a PCR assay for one or more target nucleic acid analytes and optionally to determine the level of one or more target RNA/DNA sequences within a corresponding removable sample cartridge. Typically, the processing on a sample within the corresponding removable sample cartridge involves operating the cartridge to perform a method as described herein. In certain embodiments the system is configured to contain one sample processing module. In certain embodiments the system is configured to contain at least two or more sample processing modules (e.g., at least 4, 8, 12, 16, 20, 24, 28, 32, 64, 128 or more) sample processing modules. In some embodiments, the system provides a user interface that allows the user input operational instructions and/or to monitor operation of the cartridges to determine the presence and/or quantity of one or more nucleic acids.

While the methods described herein are described primarily with reference to the GENEXPERTR cartridge by Cepheid Inc. (Sunnyvale, Calif.) (see, e.g., FIG. 1A), it will be recognized, that in view of the teachings provided herein the methods can be implemented on other cartridge/microfluidic systems, including alternative cartridge designs having valve assemblies that involve multiple interfacing components, as well as cartridge body defined by multiple interfacing components to form the multiple chambers of the cartridges, for example, those described in Korean Application No. 102293717B1 and KR102362853B1, cartridges that utilizes ultrasonic waves to lyse cells in a biological sample, for example, those described in International Application No. WO2021/245390A1, cartridges and systems that utilizes an electrowetting grid for microdroplet manipulation and electrosensor arrays configured to detect analytes of interest, for example, those described in International Application No. WO2016/077341A2, cartridges that facilitate movement of nucleic acid from one chamber to the next chamber by opening a vent pocket, for example, those described in International Application No. WO2012/145730A2, multiplexed assay systems comprising a plurality of thermocycling units such that individual chambers can be heated, cooled, and/or compressed to mix fluid within the chamber or to propel fluid in the chamber into another chamber, for example, those described in International Application No. WO2015/138343A1, and as well as systems for rapid amplification of nucleic acids facilitated by flexible portions of the sample cartridge aligned to accomplish temperature cycling for nucleic acid amplification, for example, those described in International Application No. WO2017/147085A1. Such cartridge/microfluidic systems can include, for example microfluidic systems implemented using soft lithography, micro/nano-fabricated microfluidic systems implemented using hard lithography, and the like.

In one exemplary embodiment, the cartridge can include a plurality of cartridge bodies, such as a first body, a second body, a central syringe barrel that is in fluid communication with the first body and the second body, a reaction vessel, and the like. The first body may be formed of a plurality of chambers separated from each other for reagents or buffers and sample processing. In some embodiments, the first body can be used for the purpose of storing a plurality of reagents. The second body may be formed of one or a plurality of chambers separated from each other and includes a path through which the reagent or sample from the first body moves. When the first body and the second body of cartridge are combined, a liquid flow path and optionally an air flow path can be formed between both compartments via the central syringe barrel. The liquid flow path is connected to the first body to provide a space for samples and reagents to move and mix. The air flow path may connect the reaction vessel and a vacuum control region of the “plunger” to control the vacuum that may occur when the extracted nucleic acid moves to the reaction vessel. Rotation of the syringe barrel comprising a “plunger” that can sequentially suck sample and reagents from the plurality of chambers into an interior space of the syringe barrel and discharge the mixture of the interior space into any one of the plurality of chambers (first body or second body) of the cartridge. Rotation of the syringe barrel comprising a “plunger” can suck the reagent inside the plurality of chambers of the cartridge into the interior space of the syringe barrel and then discharge the mixed reagent to a nucleic acid amplification reaction vessel.

In another exemplary embodiment, the cartridge includes a flow cover and a base plate, which together form a closed passage therein. In one embodiment of this configuration, an inner chamber containing the reagents required for dielectric extraction is provided separately from an outer chamber, and the upper and lower portions of the inner chamber are sealed. In addition, a double-structured flow cover-pad can be disposed between the outer chamber and the base plate. Closed flow paths are formed by achieving a strong coupling between the base plate—the flow cover—the pad—the outer chamber. Also provided in this configuration are beads necessary for dielectric extraction and amplification which are accommodated in a dual chamber structure of an outer chamber-bead chamber. The beads can be maintained by a dehumidifying unit positioned above the bead chamber even when the bead chamber is opened.

In further exemplary embodiment, the cartridge can include a plurality of reaction chambers, particularly, the reaction vessel can include a plurality of reaction chambers. In these embodiments, different types of lyophilized primers and probes can be provided in each reaction chamber. For example, primers and probes for viral-associated nucleic acids can be provided in one reaction chamber, and primers and probes for viral-associated nucleic acids can be provided in a second chamber for amplification and detection, and such the like. Of course, it is possible to perform various amplification and detection processes at the same time in a single reaction chamber. Accordingly, amplification of each target nucleic acid described herein may be performed individually in separate reaction chambers or wells or carried out in a multiplex reaction in a single reaction chamber or well.

Additionally, it is appreciated that the panel assay methods described herein (i.e., identification of multiple conditions based on comparative levels of multiple-target nucleic acids obtained from a single sample) can further be realized in entirely different systems, including: isothermal nucleic acid amplification systems, digital RT-PCR, electrochemical PCR, lateral flow testing cartridges, electrochemical sensors, nucleic acid sequencing, CRISPR/Cas based technologies, chemiluminescence, and nanoparticle-based colorimetric detection.

In various embodiments, the signal DNA(s) from PCR (nucleic acid amplification) reactions are amplified for detection and/or quantification. In certain embodiments, the amplification comprise any of a number of methods including, but not limited to polymerase chain reaction (PCR), ligase chain reaction (LCR), ligase detection reaction (LDR), multiplex ligation-dependent probe amplification (MLPA), ligation followed by Q-replicase amplification, primer extension, strand displacement amplification (SDA), hyperbranched strand displacement amplification, multiple displacement amplification (MDA), nucleic acid strand-based amplification (NASBA), rolling circle amplification (RCA), and the like.

In illustrative, but non-limiting embodiments, the amplification reaction may produce an optical signal that is proportional to the amount of amplified target nucleic acid (e.g., signal DNA). Illustrative optical signals include, but are not limited to a fluorescent signal, a chemiluminescent signal, an electrochemiluminescent signal, a colorimetric signal, and the like. In certain embodiments the optical signal is a fluorescent optical signal generated by a fluorescent indicator. In certain embodiments the fluorescent indicator is a non-specific intercalating dye that binds to double-stranded DNA products, while in certain other embodiments, the fluorescent indicator comprises a target sequence-specific probe (e.g., a TAQMANR; probe, a SCORPIONR; probe, a MOLECULAR BEACONR, and the like).

Single PCR reactions (nucleic acid amplification), or multiple PCR reactions (nucleic acid amplifications) run sequentially (or simultaneously in separate temperature controlled channels or chambers) can also use the same detectable label since sequentially run PCR signal DNAs are analyzed sequentially and the simultaneous PCR signal DNAs are distinguished by the occurrence in different temperature controlled channels or chambers. The signal produced by this amplification can be distinguished from other amplification products because it is not run at the same time and/or because it is run in a different reaction channel/chamber. However, where multiple nucleic acid amplifications are run simultaneously in the same chamber the reaction products of for each analysis are typically detected and/or quantified by the use of different and distinguishable labels.

In certain embodiments, amplification products (amplified nucleic acid from nucleic acid analysis) can be detected using methods well known to those of skill in the art. In certain embodiments the amplification is a straightforward simple PCR amplification reaction. In certain embodiments, however, a nested PCR reaction is used to amplify the nucleic acid from the nucleic acid analysis. In various embodiments, multiplexed PCR assays are contemplated, particularly where it is desired to analyze multiple products of the nucleic acid analysis in the same amplification reaction. In certain embodiments in such multiplexed amplification reactions, each probe (e.g., for each specific analyte) has its own specific dye/fluor so that it is detectable independently of the other probes. In certain embodiments, typically, for signal generation, the probes used in various amplification reactions utilize a change in the fluorescence of a fluorophore due to a change in its interaction with another molecule or moiety brought about by changing the distance between the fluorophore and the interacting molecule or moiety for detection and/or quantification of the amplified product. Alternatively, other methods of detecting a polynucleotide in a sample, including, but not limited to, the use of radioactively labeled probes, are contemplated.

### Assay Workflows

Prior to carrying out amplification reactions on a sample, one or more sample preparation operations are performed on the sample. Typically, these sample preparation operations will include such manipulations as extraction of intracellular material, e.g., nucleic acids from whole cell samples, viruses and the like to form a crude extract, additional treatments to prepare the sample for subsequent operations, e.g., denaturation of contaminating (e.g., DNA binding) proteins, purification, filtration, desalting, and the like. Liberation of nucleic acids from the sample cells or viruses, and denaturation of DNA binding proteins may generally be performed by chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within a sample preparation chamber, a separate accessible chamber, or may be externally introduced. Preferably, sample preparation is carried out in only one step or no more than two steps. For example, sample preparation can include heating the sample in a lysis solution without further purification prior to carrying out the amplification reaction. In some embodiments, the lysed sample may be diluted prior to carrying out the amplification reaction. One or more of these various sample preparation operations are readily incorporated into the fluidly closed cartridge systems contemplated herein.

In one aspect, the assay sample cartridge, as described herein, is capable of a specialized workflow that performs lysing and detection of differing target analytes as required for a particular panel assay. In some embodiments, the cartrdige is configured for chemical lysing of the multiple target organisms. In other embodiments, the cartridge is configured for mechanical lysing of the multiple target organisms. In still other embodiments, the cartridge is configured for both mechanical and chemical lysing to allow lysing of multiple targets of differing types. Accordingly, the sample cartridge can be configured to perform the panel assay by an existing workflow associated with conventional cartridges, or can be operated according to an new workflow specially configured for the panel assay.

In one aspect, the sample cartridge having a valve assembly as described in FIG. 3D herein, is capable of a variety of workflows that perform: chemical lysing of target organisms, mechanical lysing of target organisms, or both. Accordingly, the sample cartridge can perform an existing workflow associated with conventional specialized cartridges or can perform entirely new workflows that perform both.

Exemplary assay workflows A-C(below) can be performed with a single universal cartridge. In any of these embodiments, the filter can be formed of glass filter to promote affinity binding of the nucleic acids (NA) to the glass fibers and a pore size suited for chemical lysing as well. In any of these workflows, the nucleic acid amplification can be PCR, real-time PCR, isothermal amplification (including but not limited to nucleic acid sequence-based amplification, loop-mediated isothermal amplification, helicase-dependent amplification, rolling circle amplification, multiple displacement amplification, whole genome amplification or recombinase polymerase amplification) or other nucleic acid amplification methods known to persons of skill in the art.

In Workflow A, the sample is optionally exposed to a sample treatment or chemically lysed, then the treated or lysed fluid sample is flowed through the filter where target organisms are captured. In some embodiments, the sample treatment is used to either weaken the cell wall or to inactivate the sample or make it less viscous to facilitate being processed through the filter. The filter is then washed, leaving the target organisms on the filter. Next, the target organisms are mechanically lysed, such as by sonication, to release nucleic acid (NA). In some embodiments, mechanical lysing includes in-filling glass beads along the filter to aid in mechanical lysing of the target. Next, the NA is eluted from the filter and then nucleic acid amplification is performed is performed.

In Workflow B, the sample is chemically lysed to obtain the NA targets. In some embodiments, after chemically lysing, the NA is bound to the filter by the presence of precipitating and binding reagent. Next, the filter is washed with a rinse reagent while the NA remains bound to the filter. Typically, the wash reagents have some amount of salt which still promotes the binding of the NA to the filter, while allowing removal of non-target materials. Next, the filter is eluted to remove the NA targets. In some embodiments, the elution is performed with a pH neutral buffer or basic buffer fluid. The target NA is then delivered to an attached reaction vessel to perform nucleic acid amplification.

In Workflow C, the fluid sample is exposed to sample treatment and/or chemically lyse the target organisms. Next, the NA freed by chemical lysing is bound to the filter. This step may utilize precipitating and binding reagent. Next, the filter is washed with a rinse reagent while the NA remains bound to the filter. Typically, the wash reagents have some amount of salt which still promotes the binding of the NA to the filter, while allowing removal of non-target materials. Next, the target organisms captured in the filter are heat and/or mechanically lysed. This may utilize sonication, and may further utilize glass beads to facilitate mechanical lysing of select target organisms. Then, the lysed target NA is eluted from the filter. In some embodiments, the elution is performed with a pH neutral buffer or basic buffer fluid. The target NA is then delivered to an attached reaction vessel to perform nucleic acid amplification. Thus, in this workflow, the workflow allows for lysing of multiple differing target organisms, some requiring only chemically lysing (e.g. viral targets), and others requiring mechanical lysing (e.g. bacteria, spores, etc.), such that all these target NAs can be released from a single sample and tested by the same sample cartridge. While the above workflow described mechanical lysing after chemical lysing, it is appreciated that other workflows may be utilized in which chemical lysing occurs after mechanical lysing.

In some embodiments, the sample cartridge includes an identifier with information as to the appropriate workflow needed for a particular panel of assays, so that an instrument module receiving the sample cartridge operates according to the specified workflow:

### Exemplary Assay Configurations

Reagents for MPOX Clade II biomarker Panel Assay

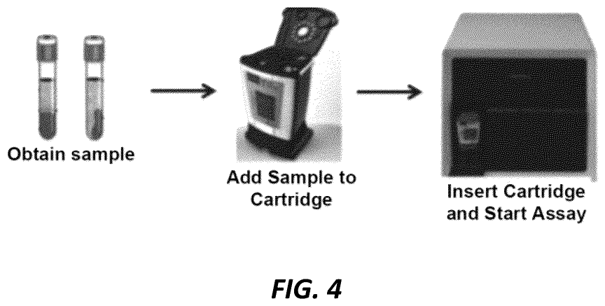

FIG. 5 shows an exemplary sample cartridge than can be used as part of the GENEXPERTR system, with the various chambers designated by number. The exemplary cartridge can include: lyophilized reagents in the form of one or more beads as described herein: an optional lysis reagent: alkaline agent, optional binding reagent, filtering reagent, washing reagent, and eluting reagent. The last four reagents are named according to the functions they perform with respect to nucleic acid. Thus, for example the binding reagent facilitates the binding of nucleic acid to a substrate, the filtering reagent facilitates filtration of the nucleic acid.

In some embodiments, the lysis reagent can include a chaotropic agent, a chelating agent, a buffer, an alkaline agent, or a detergent. The chaotropic agent can be selected from a guanidinium compound such as guanidinium thiocyanate or guanidinium hydrochloride, an alkali perchlorate such as lithium perchlorate, an alkali iodide, magnesium chloride, urea, thiourea, a formamide, or a combination thereof. The concentration of the chaotropic agent can range from about 1 M to about 10 M, such as from about 2.5 M to about 7.5 M, or less than 4.5 M, less than 2 M, or less than 1 M. The chelating agent can be selected from N-acetyl-L-cysteine, ethylenediaminetetraacetic acid (EDTA), diethylene triamine pentaacetic acid (DTPA), ethylenediamine-N,N′-disuccinic acid (EDDS), 1,2-bis(o-aminophenoxy)ethane-N,N,N′,N′-tetraacetic acid (BAPTA), and a phosphonate chelating agent. The concentration of the chelating agent can range from about 10 mM to about 100 mM and/or comprises about 0.5% to about 5% of the lysis reagent. The buffer can be selected from the group consisting of Tris, phosphate buffer, PBS, citrate buffer, TAPS, Bicine, Tricine, TAPSO, HEPES, TES, MOPS, PIPES, Cacodylate, SSC, and MES. The concentration of the buffer can range from about 5 mM to about 100 mM, such as from about 5 mM to about 50 mM. The detergent can be selected from an ionic detergent or a non-ionic detergent. In some examples, the detergent comprises a detergent selected from the group consisting of N-lauroylsarcosine, sodium dodecyl sulfate (SDS), cetyl methyl ammonium bromide (CTAB), TRITON®-X-100, n-octyl-β-D-glucopyranoside, CHAPS, n-octanoylsucrose, n-octyl-β-D-maltopyranoside, n-octyl-β-D-thioglucopyranoside, PLURONIC′R: F-127, TWEENR 20, and n-heptyl-β-D-glucopyranoside. The detergent can comprise about 0.1% to about 2% of the lysis reagent, and/or ranges from about 10 mM up to about 100 mM. The lysis reagent can have a pH ranging from about pH 3.0 to about pH 5.5.

In some embodiments, the assays disclosed herein do not utilize a chaotropic agent or a lysis buffer. When a chaotropic agent or lysis buffer is not used, the sample can be contacted with a buffer (or filtering reagent) including, for example, saline (including one or more inorganic salts, such as CaCl₂), MgSO₄, KCl, NaHCO₃, NaCl, etc.), phosphate buffer, Tris buffer, 2-amino-2-hydroxymethyl-1,3-propanediol, HEPES, PBS, citrate buffer, TES, MOPS, PIPES, Cacodylate, SSC, MES, saccharide or disaccharide, or combinations thereof. For example, the buffer can be a commercially available buffer such as Hanks' Balanced Salt Solution available from Sigma Aldrich or TE Buffer available from Fisher BioReagents.

In some embodiments, the alkaline agent can be selected from an alkali metal hydroxide, such as sodium hydroxide or potassium hydroxide. The concentration of the alkaline agent can be about 0.5 N to 5 N.

The binding reagent can promote binding of nucleic acids to the filter, facilitating the removal of non-target material. In some embodiments, the binding reagent can include a binding polymer such as polyacrylic acid (PAA), polyacrylamide (PAM), polyethylene glycol (PEG), poly(sulfobetaine), or a salt, or combinations thereof. In some embodiments, the filtering reagent and/or the washing reagent can include the binding reagent. For example, the binding reagent, the filtering reagent, and/or the washing reagent can include a binding polymer (e.g., PEG 200), buffer, inorganic salt, antioxidant and/or chelating agent, antifoam SE15, sodium azide, disaccharide or disaccharide derivative, carrier protein, detergent, or DMSO. The binding polymer can be present in an amount of at least 10% v/v, at least 20% v/v, at least 30% v/v, and/or less than 60% v/v, less than 40% v/v; less than 30% v/v, less than 20% v/v, or less than 10% v/v or can fall within any range bounded by any of these values, e.g., from 10% to 60% v/v, of the binding reagent, filtering reagent, and/or the washing reagent. The buffer can be selected from the group consisting of Tris, 2-amino-2-hydroxymethyl-1,3-propanediol, HEPES, phosphate buffer, PBS, citrate buffer, TAPS, Bicine, Tricine, TAPSO, HEPES, TES, MOPS, PIPES, Cacodylate, SSC, and MES. The concentration of the buffer can range from about 5 mM to about 100 mM, such as from about 5 mM to about 50 mM. The salt, such as NaCl, KCl, or MgCl₂, can be present at a concentration from about 0.05 M to about 1 M, such as from about 0.1 M to about 0.5 M. The antioxidant and/or chelating agent comprises an agent selected from the group consisting of N-acetyl-L-cysteine, ethylenediaminetetraacetic acid (EDTA), diethylene triamine pentaacetic acid (DTPA), ethylenediamine-N,N′-disuccinic acid (EDDS), 1,2-bis(o-aminophenoxy)ethane-N,N,N′,N′-tetraacetic acid (BAPTA), and a phosphonate chelating agent. In some embodiments the antioxidant and/or chelating agent comprises EDTA. In certain embodiments the antioxidant and/or chelating agent comprise 0).2% to about 5%, about 0).2% to about 3%, or about 0).5% to about 2%, or about 0.5% of the binding reagent, filtering reagent, and/or the washing reagent. In some embodiments the concentration of the antioxidant and/or chelating agent in the binding reagent, filtering reagent, or the washing reagent ranges from about 2 mM to about 50 mM or about 5 mM to about 20 mM. In some embodiments, the detergent is an ionic detergent or a non-ionic detergent. The detergent can be selected from an ionic detergent or a non-ionic detergent. In some examples, the detergent comprises a detergent selected from the group consisting of N-lauroylsarcosine, sodium dodecyl sulfate (SDS), cetyl methyl ammonium bromide (CTAB), TRITON®-X-100, n-octyl-β-D-glucopyranoside. CHAPS, n-octanoylsucrose, n-octyl-β-D-maltopyranoside, n-octyl-β-D-thioglucopyranoside, PLURONIC R: F-127. TWEENR: 20, Brij-35, and n-heptyl-β-D-glucopyranoside. The detergent can comprise about 0).1% to about 2% of the binding reagent, filtering reagent, and/or the washing reagent, and/or ranges from about 10 mM up to about 100 mM. The binding reagent, filtering reagent and/or the washing reagent can have a pH ranging from about pH 6.0 to about pH 8.0 (such as from about 6.5 to about 7.5).

In some embodiments, the eluting reagent can have a pH greater than about 9, greater than about 10, greater than about 11, or greater than about 12. The use of high pH to elute nucleic acid such as DNA is unique especially to the cartridges described herein and provides improved speed and performance of the disclosed methods. Speed is provided by the rapid neutralization of acidic ammonium ions by the high concentration of hydroxide ions. Alkylamines have a pKa˜10-11 and are immediately deprotonated at pH 12.7, to form the neutral free base on the solid surface, and release the cationic DNA. A further advantage of the high pH is the denaturing effect of KOH on captured DNA or RNA. Acidic functional groups in the heterocyclic bases of DNA or RNA are immediately deprotonated and cannot form Watson-Crick bonds. Double-stranded structures and other secondary structures are disrupted, but can re-nature when neutralized for example, with Tris HCl. This chemical denaturing of captured genomic DNA can be an advantage for isothermal assays that do not undergo the usual heat denaturing of PCR. The cartridges provided herein allow for rapid neutralization of eluted DNA or RNA in KOH followed by reaction with Tris to produce a final pH of about 8.5 for downstream PCR or other nucleic acid assays. In some embodiments, the eluting reagent can have a pH less than about 9, less than about 8.5, or less than about 8. This lower-pH elution of bound DNA or RNA can be an advantage, especially for devices that don't facilitate rapid neutralization of the KOH solution. It is known that RNA is hydrolyzed by high pH, but short exposure times to KOH can provide for good quality RNA. In some examples, the eluting reagent comprises a polyanion, a polycation, ammonia or an alkali metal hydroxide. For example, the eluting reagent may comprise a polyanion such as a carrageenan, a carrier nucleic acid, or a combination thereof.

In some instances, to reduce bubble formation in one or more of the chambers, the detergent Brij may be added to one or more of the reagents described herein.

It is understood that various other reagents and initial volumes can be used for performing an automated PCR panel assay on a sample inserted into the cartridge.

### Specialized Cartridge Components

FIG. 5 illustrates an exemplary cartridge suitable for performing a multi-target panel assay, as described herein. The illustrated cartridges are based on the GENEXPERT® cartridge (Cepheid, Inc., Sunnyvale, Calif.). The cartridge 100 comprises a cartridge body 102 having multiple chambers 108 defined therien for holding various reagent and/or buffers. The chambers are disposed around a central syringe barrel 109 that is in fluid communication with valve body 110 through valve syringe tube 106 extending through the syringe barrel 109. In some embodiments, the valve assembly include any of the valve assemblies described herein, or any suitable valve assembly. In this embodiment directed to an cartridge, the cartridge utilizes a valve assembly capable of chemical lysing, for example, any of valve assemblies B-D in FIG. 3. The valve body 110 is interface within the cartridge body and supported on a cartridge base 101. The cartridge typically contains one or channels or cavities that can contain a filter material (e.g. glass filter column) as described herein that can function to bind and elute a nucleic acid. In various embodiments. the cartridge further comprises one or more temperature controlled channels or chambers that can, in certain embodiments, function as thermocycling chambers. A “plunger” not shown can be operated to draw fluid into the syringe barrel 109 and rotation of the valve body/syringe tube provides selective fluid communication between the various reagent chambers and channels, reaction chamber(s). Thus, the various reagent chambers, reaction chambers, matrix material(s), and channels are selectively in fluid communication by rotation of the valve and plunger and reagent movement (e.g., chamber loading or unloading) is operated by the “syringe” action of the plunger. The attached reaction vessel 116 provides optical windows to provide real-time detection of, e.g., amplification products, base identity in sequencing operations, by operation of the module within the system described herein.

While the methods described above are described with respect to specific chambers in the GENEXPERTR; cartridge, it will be recognized that the particular reagent/chamber assignments can be varied depending on the particularities of the nucleic acid detection/quantification assay. It will also be recognized that in certain embodiments. variants of the GENEXPERTR cartridge are also contemplated. Such variants can include, but are not limited to, more reagent chambers or fewer reagent chambers and/or different sized chambers, two (or more) sample receiving chambers, two (or more) temperature controlled channels or chambers, stacked cartridges (providing control of two cartridges by one module), and the like. In one aspect, the sample cartridge includes one or more features or components that are specially configured per the unique requirements of a particular multi-target assay. In this embodiment, the sample cartridge utilizes certain components specifically developed for the MPX Clade II Panel assay.

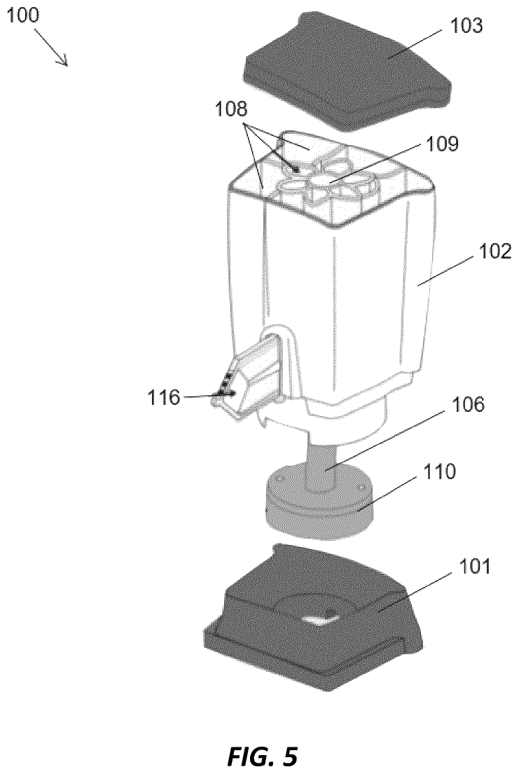

FIGS. 5A-6B show a detailed view of a valve assembly for use in a multi-target panel assay cartridge, in accordance with some embodiments. In this embodiment, the valve assembly is configured for chemical lysing. The valve assembly includes a valve body 110 having a disk shape and tubular syringe type 106 along a proximal portion thereof. The disk portion has a generally planar external upper port surface 120 having one or more ports therein (e.g., 142, 146), as shown in FIG. 6A. The valve assembly is rotatable relative to the cartridge housing, for example by engagement of an interface feature 123 with a driven mechanism of the module. The ports 142, 146 on the top planar surface permit fluid communication between the syringe tube 106 and each of the chambers within the cartridge and/or one or more channels. In this embodiment, the ports 142, 146 interface with a circular gasket 126 having multiple ports defined therein which interfaces and fluidically couple with corresponding ports on the bottom of the chambers in the cartridge body. Upon rotation of the valve assembly, the top ports 142, 146 interface with differing pairs of ports so as to fluidically couple the various chambers to each other or to channels of the reaction vessel attached to the cartridge so that movement of the “plunger” through the syringe tube advances fluid between the various chambers and/or channels to facilitate processing and subsequent analytical testing of the fluid sample.

As seen in FIG. 6B, the valve body 110 includes a lysing chamber 121 on an underside thereof that may contain solid phase material for capturing cells, spores, viruses, or microorganisms to be lysed. Suitable solid phase materials include, without limitation, filters, beads, fibers, membranes, filter paper, glass wool, polymers, or gels. One or more interior ports facilitate fluid flow into and out of the lysing chamber. In this embodiment, the solid phase material is a glass filter 114 that resides in a filter recess 111 in the valve body 110 for capturing nucleic acid released by chemical lysing of the target organisms in the fluid sample.

As shown in FIGS. 6A-6B, an outer wall 122 of the valve body encloses the lysing chamber 121 along with bottom cap 112. As seen in FIG. 6B, the lysing chamber 121 includes a pair of processing ports to facilitate flow of fluid sample into and out of the lysing chamber. A first fluid processing port 124 coupled to a first fluid processing channel defined within the valve body, and a second fluid processing port 125 coupled to a second fluid processing channel defined within the valve body. Through these channels, the first fluid processing channel is coupled to both the inner conduit of the syringe tube 106 and external port 142, while the second fluid processing channel is coupled to the second external port 146. By this configuration, application of pressure by movement of a “plunger” through the syringe tube 106 acts like a syringe, drawing fluid from one chamber into the syringe tube, and after rotation of the valve assembly to position the respective ports to couple with another chamber, movement of the plunger advances the fluid through the valve body and channels into another chamber or channel to the reaction vessel. This approach can perform the various processing steps required to perform a sample preparation protocol for the targeted analytes of the multi-target panel assay.

In one aspect, the assay described herein performs chemical lysing of the targeted viruses. Broadly applicable lysing buffers may have elevated alkalinity (e.g., sodium hydroxide). Further high alkalinity eluting buffers (e.g., ammonia or an alkali metal hydroxide) may be used to elute the nucleic acids bound to the glass filter. While these buffers allow for chemical preparation of the sample, in practice, use of such buffers can be problematic in conventional cartridge as these high alkalinity buffers can degrade the valve assembly material and sealing interfaces between cartridge components, resulting in cracking of the valve assembly and leakage during processing. Such leakage can be detrimental to sample processing. A specialized valve assembly can be used to resist elevated alkalinity (e.g., greater than pH of 10, greater than pH 11, or greater than pH 12) of these buffers. One difficulty in developing conventional valve assemblies is that these valve assemblies are fabricated by injection molding of certain polymer materials, typically polycarbonate, polyolefin (including polyethylene or polypropylene), or combinations thereof. Typical such polymer materials used in injection molding are not resistant to the range of elevated alkalinity noted, and polymer materials that are resistant, may be considerably more costly and may be less suited for injection molding of small-scale microfluidic features. Thus, the specialized valve assemblies can be developed by annealing valve body assemblies formed with conventional polymers in order to harden the polymer material sufficiently to resist elevated alkalinity. Annealing of polycarbonate reduces chemical corrosion by the mixture of NaOH and GTC within the lysis reagent and the eluting reagent. In some embodiments, after the valve assemblies are formed, they are heated to an elevated temperature (e.g., 90-100° C., about 100° C.)for about an hour or more, then allowed to slowly cool in a temperature-controlled manner for at least 30 minutes. Studies showed that these annealed valve assemblies resulted in valve assemblies of substantially the same design and material as conventional valve assemblies, except they were resistant to high alkalinity buffers that could not have been feasible to use in conventional cartridges. Testing of the annealed valve body to alkaline resistance can be performed by exposing the valve body assembly (VBA) to NaOH/GTC, followed by visual inspection for cracking of VBA after 30 min or so. It is appreciated that this annealed cartridge can be advantageous for various reasons and need not be tied to any particular assay described herein. Moreover, it is further appreciated that the assays described herein may be performed with various other cartridge designs, devices and systems and need not be tied to the particular cartridge designs described herein.

### Exemplary Detection Methods, Results, and Handling of Results

In some embodiments, a computer-based analysis program is used to translate the raw data generated by the detection assay into data of predictive value for a clinician.

Melt Curve analysis can be evaluated by GENEXPERTR Software to determine the presence of PCR product. Melting temperature (Tₘ) and Melt peak height of the curve is calculated automatically by the analysis software. The melt curve is detected as positive if Tₘ falls inside the valid Tₘ range specified for each target nucleic acid. The melt curve is called as negative if melt curve is not in the appropriate Tₘ range. The software automatically calculates the cycle threshold (Ct), Endpoint and Probe check values. Illustrative limit-of-detection concentrations, Ct cut-offs, and methods for determining the same are provided in the examples below.

Before the start of the PCR reaction, the GENEXPERTR System measures the fluorescence signal from the probes to monitor bead rehydration, reaction tube filling, probe integrity, and dye stability. This Probe Check Control (PCC) passes if it meets validated acceptance criteria.

In some embodiments, a computer-based analysis program is used to translate the raw data generated by the detection assay into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

When the GENEXPERT® System is used, the results are interpreted automatically and are shown in a “View Results” window. Positive targets are highlighted in red color, negative targets are highlighted in green color, and indeterminate targets are highlighted in light gray color. Samples with coinfection may appear with positives results for multiple targets. Invalid, Error or No result are highlighted in light gray color.

In some embodiments. the results and interpretation are provided in Table 3-1 to 3-2.

TABLE 3-1
MPOX Possible Results

| Result | MPXV | OPXV | SAC | SPC |
|---|---|---|---|---|
| MPXV Clade II DETECTED | + | +/− | +/− | +/− |
| Non-Variola OPXV | − | + | +/− | +/− |
| DETECTED | | | | |
| MPXV Clade II NOT | − | − | + | + |
| DETECTED, Non-Variola | | | | |
| OPXV NOT DETECTED | | | | |
| INVALID | − | − | − | − |
| | − | − | − | + |
| | − | − | + | − |

TABLE 3-2
Interpretation of Test Results for the MPOX Possible Results

| Result | Interpretation |
|---|---|
| MPXV Clade II | MPOX Clade II Viral DNA DETECTED |
| DETECTED | This specimen is POSITIVE for MPOX virus |
| | (West African clade, clade II). |
| | Laboratories should notify appropriate |
| | Federal, State, or local public health |
| | agencies of the test results in accordance |
| | with applicable laws. |
| Non-Variola | Non-Variola Orthopox Viral DNA DETECTED. |
| OPXV DETECTED | This specimen is POSITIVE for non-variola |
| | Orthopoxvirus DNA. |
| | Low levels of viral DNA in a specimen may |
| | on rare occasions, result in MPOX virus |
| | (West African clade, clade II) DNA Not |
| | Detected and non- variola Orthopoxvirus |
| | DNA Detected. This result may occur in the |
| | case of low viral DNA concentration at |
| | levels below the limit of detection or may |
| | indicate another Orthopoxvirus infection |
| | although no other known Orthopoxviruses |
| | are currently circulating in the United |
| | States. |
| | If clinically indicated, consider |
| | collecting another specimen. |
| | Laboratories should notify appropriate |
| | Federal, State, or local public health |
| | agencies of the test results in accordance |
| | with applicable laws. |
| MPXV Clade II | MPOX Clade II Viral DNA and Non-Variola |
| NOT DETECTED, | Orthopox Viral DNA not detected. |
| Non-Variola OPXV | This specimen is NEGATIVE for MPOX virus |
| NOT DETECTED | DNA. If clinically indicated, consider |
| | collecting another specimen. |
| NO RESULT | If the result is NO RESULT, repeat the test |
| | with a new cartridge. |
| INVALID | If the result is INVALID repeat the test, |
| | if the second test is INVALID collect a |
| | new specimen |
| ERROR | The result is an instrument error or |
| | cartridge defect. Repeat the test using |
| | a new cartridge in a different module |

If the results of the test targets (MPOX or non-variola Orthopoxvirus) are positive, the test can be considered valid regardless of the results for the SPC and SAC.

Exemplary detection methods, results, and handling of results for host biomarker targets are described in US Patent Publication No. 2022/0298572, which is incorporated by reference for this description.

The present disclosure contemplates any method capable of receiving, processing, and transmitting the information to and from laboratories conducting the assays, information provides, medical personal, and subjects. For example, in some embodiments of the present invention, a sample is obtained from a subject and submitted to a testing service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample collected and sent to the testing service, or subjects may collect the sample themselves and directly send it to a testing service. Where the sample includes previously determined biological information, the information may be directly sent to the testing service by the subject (e.g., an information card containing the information may be scanned by a computer and the data transmitted to a computer of the profiling center using an electronic communication systems). Once received by the testing service, the sample is processed and a set of test results is produced, specific for the diagnostic or prognostic information desired for the subject.

The test results can be prepared in a format suitable for interpretation by a treating clinician. For example, rather than providing raw expression data, the prepared format may represent a diagnosis or risk assessment for the subject, with or without recommendations for particular treatment options. The test results may be displayed to the clinician by any suitable method. For example, in some embodiments, the testing service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

### Kits

Also contemplated is a kit for carrying out the methods described herein. Such kits include one or more reagents useful for practicing any of these methods. A kit generally includes a package with one or more containers holding the reagents, as one or more separate compositions or, optionally, as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits preferably include instructions for carrying out one or more of the screening methods described herein. Instructions included in kits can be affixed to packaging material or can be included as a package insert. While the instructions are typically written or printed materials, they are not limited to such. Any medium capable of storing such instructions and communicating them to an end user can be employed. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In some embodiments, a kit includes primer pairs for amplifying and/or detecting the above-described MPXV (e.g., MPX Clade II and non-variola MPXV) biomarker panel targets described above, optionally with probes specific for these targets. Such kits can additionally include primers pairs and optional probes for detecting one or more of the above-described host biomarker targets. In some embodiments, these kits can include primers pairs and optional probes for detecting one or more of the above-described controls

In some embodiments, the kit can include any the reagents described above provided with or in one or more GENEXPERT® cartridge(s). See e.g., U.S. Pat. Nos. 5,958,349, 6,403,037, 6,440,725, 6,783,736, 6,818, 185; each of which is herein incorporated by reference for this description). In some embodiments, reagents for measuring detecting MPXV (e.g., MPX Clade II and non-variola MPXV) and for detecting host biomarkers are be provided in separate cartridges within a kit.

Any of the kits described here can include, in some embodiments, a receptacle for a sample and/or a swab for collecting a sample.

### EXAMPLES

### Example 1: MPOX Assay Designs to Distinguish Between Clade II and Clade I

The following approach was taken to obtain primer/probe sets that are specific for MPOX (MPX or MPOX) Clade II and that do not detect Clade I.

### Datasets and Methods

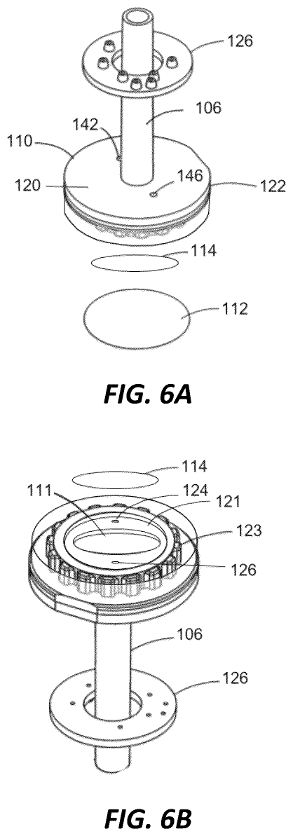

The sequences and metadata were those used in the NextStrain analysis shown in FIG. 7. The sequences are all from NCBI (for MPX the NCBI and GISAID datasets appear to be effectively equivalent).

The clade assignments are taken from the NextStrain metadata. Their site indicates a set of specific nucleotide differences that define the clades. In addition, they exclude terminal regions of the genomes from their analysis. Specifically, they describe “masking several regions of the genome, including the first 1500 and last 7000 base pairs and a repetitive region of variable length.” The terminal regions include repeats. Not all sequences in the dataset have an assigned clade.

The initial dataset consists of 2,103 MPX sequences, of which 152 are assigned to Clade I. 1,712 are assigned to Clade II, and 149 are unassigned. Of the Clade II genomes, 107 are Clade IIa, 1,542 are Clade IIb, and 62 are not assigned to a subclade.

All Clade I and Clade II genomes were formatted as BLAST databases. GenBank sequence NC_063383.1 (Clade IIb) was used as the reference sequence. This sequence was split into 150 nucleotide segments, overlapping each other by 50 nucleotides. These were compared to the Clade I and Clade II datasets to identify segments that are both highly conserved within Clade II and show no, or minimal similarity to Clade I.

A set of candidate segments was selected and these were then compared against a small set of non-MPX Orthopoxvirus genomes in order to identify any that had no, or low, similarity to non-MPX species.

Two good candidates were identified and candidate primer/probe sets were designed. These are shown below.

In the Clade II alignments, the last number in each sequence label is the number of isolates with that unique amplicon sequence. Differences from the most frequent sequence are capitalized and bolded.

Multiple alternate designs for each oligo are provided.

Neither amplicon has a significant match to a Clade I genome. In the matches to the OPX subset, there are a number of insertions/deletions relative to the Clade II amplicon which are capitalized and bolded.

Neither amplicon has a significant match to any other species outside the Orthopoxviruses.

Candidate #1-coordinates NC_063383.1_133825-133927
In coding region of gene OPG153-Orthopoxvirus A26L/A30L protein
Clade II matches

| | |
|---|---|
| | 1 |
| U84503_2268_2166_1549 | gttcaagatccctgattctagtctttagcctatttacctcctcagaagatgctccgttaccgttttta |
| OP245318_133040_133142_3 | gttcaagatccctgattctagtctttagcctatttacctcctTagaagatgctccgttaccgttttta |
| OX044345_133966_134068_3 | gttcaagatccctgattctagtctttagcctatttacctTctcagaagatgctccgttaccgttttta |
| OX044338_133966_134067_2 | gttcaagatccctgattctagtctttagcctatttacctcctcagaagatgctccgttaccgttttta |
| mpx_cld2_1_fwd1 63.8 | gttcaagatccctgattctagtctt (SEQ ID NO: 37) |
| mpx_cld2_1_fwd2 63.3 | ttcaagatccctgattctagtcttt (SEQ ID NO: 38) |
| mpx_cld2_1_fwd3 62.6 | tcaagatccctgattctagtcttta (SEQ ID NO: 39) |
| mpx_cld2_1_fwd4 62.1 | caagatccctgattctagtctttag (SEQ ID NO: 40) |
| mpx_cld2_1_fwd5 63.7 | aagatccctgattctagtctttagc (SEQ ID NO: 41) |
| mpx_cld2_1_prb1 68.6 | ctcagaagatgctccgttaccgttt |
| mpx_cld2_1_prb2 68.3 | tcagaagatgctccgttaccgtttt |
| mpx_cld2_1_prb3 67.5 | cagaagatgctccgttaccgttttt |
| mpx_cld2_1_prb4 66.3 | agaagatgctccgttaccgttttta |
| mpx_cld2_1_rev1 61.9 | |
| mpx_cld2_1_rev2 63.0 | |
| mpx_cld2_1_rev3 62.1 | |
| mpx_cld2_1_rev4 60.8 | |
| mpx_cld2_1_rev5 61.1 | |
| | 103 |
| U84503_2268_2166_1549 | caatcgttaagatgtctatcaagatccatgattct |
| OP245318_133040_133142_3 | caatcgttaagatgtctatcaagatccatgattct |
| OX044345_133966_134068_3 | caatcgttaagatgtctatcaagatccatgattct |
| OX044338_133966_134067_2 | caatcgttaagatgtctatcaagatccatgattc- |
| mpx_cld2_1_fwd1 63.8 | |
| mpx_cld2_1_fwd2 63.3 | |
| mpx_cld2_1_fwd3 62.6 | |
| mpx_cld2_1_fwd4 62.1 | |
| mpx_cld2_1_fwd5 63.7 | |
| mpx_cld2_1_prb1 68.6 | (SEQ ID NO: 42) |
| mpx_cld2_1_prb2 68.3 | (SEQ ID NO: 43) |
| mpx_cld2_1_prb3 67.5 | (SEQ ID NO: 44) |
| mpx_cld2_1_prb4 66.3 | (SEQ ID NO: 45) |
| mpx_cld2_1_rev1 61.9 | atcgttaagatgtctatcaagatcc (SEQ ID NO: 46) |
| mpx_cld2_1_rev2 63.0 | tcgttaagatgtctatcaagatcca (SEQ ID NO: 47) |
| mpx_cld2_1_rev3 62.1 | cgttaagatgtctatcaagatccat (SEQ ID NO: 48) |
| mpx_cld2_1_rev4 60.8 | gttaagatgtctatcaagatccatg (SEQ ID NO: 49 |
| mpx_cld2_1_rev5 61.1 | ttaagatgtctatcaagatccatga (SEQ ID NO: 50) |

U84503_2268_2166_1549 (SEQ ID NO: 51); OP245318_133040_133142_3 (SEQ ID NO: 52); OX044345_133966_134068_3 (SEQ ID NO: 53); OX044338_133966_134067_2 (SEQ ID NO: 54).

The sequences identified as U84503 (position 2268_2166), OP245318 (position 133040_133142), OX044345 (position 133966_134068), and OX044338 (position 133966_134067) are from Clade II genomes (obtained from NCBI GenBank). The capitalized and bolded letters are mismatches in the genome sequence compared to the coding region of gene OPG153 of MPXV Clade II (deposited in NCBI Genbank as NC_063383.1). The sequences identified mpx_cld2_1_fwd, mpx_cld2_1_prb, and mpx_cld2_1_rev represent possible forward primers, probes, and reverse primers that can be used to detect Clade II in a MPX assay.

The above shows no significant matches with Clade I. Therefore, this amplicon lies within a larger segment that is not present in Clade I.

Candidate #1-OPX matches*

| | |
|---|---|
| | 1 |
| MPX Clade IIb Reference | gttcaagatccctgattctagtctttagcctatttacctcctcagaagatgctccgttaccgtt---ttta |
| NC_055231.1_144914_145019 | gttcaagatccctgattctagtctttagcctatttacctcctcagaagatgAtccgttaccgttGcgttta |
| NC_008291.1_135985_136090 | gttcaagatccctgattctagtctttagcctatttacctcctcagaagatgTtccgttaccgttGcgttta |
| NC_003663.2 152956_153061 | gttcaagatccctgattctagtctttagcctatttacctcctcagaagatgTTtccgttaccgtCGcgtttt |
| NC_006998.1_136036_136141 | gttTTaagatTcctgattctagtctttagcctatttacctcctcagaagatgTtccgttaccgttGcgttt |
| NC_001611.1_126867_126972 | gttTaagatcActAattctagtctttagcctatttacctcctcagaagatgTtccgttaccgttGcgttta |
| NC_003391.1_138318_138401 | ----------------------ctttaAAcctatttacctcctcagaagatgTtccgttaccgttGcgttt |
| NC_055230.1_151168_151270 | gttcTagatcTctgatAcGGaCtctttagcctatCtacctcTtcTgaagatgAtcc---accgttGcgttt |
| NC_004105.1_144647_144731 | ---------------------tctttagcctatCtaTTtcctcagaagatgAtccAttaccgttAcgtCta |
| mpx_cld2_1_fwd1 63.8 | gttcaagatccctgattctagtctt (SEQ ID NO: 55) |
| mpx_cld2_1_fwd2 63.3 | ttcaagatccctgattctagtcttt (SEQ ID NO: 56) |
| mpx_cld2_1_fwd3 62.6 | tcaagatccctgattctagtcttta (SEQ ID NO: 57) |
| mpx_cld2_1_fwd4 62.1 | caagatccctgattctagtctttag (SEQ ID NO: 58) |
| mpx_cld2_1_fwd5 63.7 | aagatccctgattctagtctttagc (SEQ ID NO: 59) |
| mpx_cld2_1_prb1 68.6 | ctcagaagatgctccgttaccgtt---t |
| mpx_cld2_1_prb2 68.3 | tcagaagatgctccgttaccgtt---tt |
| mpx_cld2_1_prb3 67.5 | cagaagatgctccgttaccgtt---ttt |
| mpx_cld2_1_prb4 66.3 | agaagatgctccgttaccgtt---ttta |
| mpx_cld2_1_rev1 61.9 | |
| mpx_cld2_1_rev2 63.0 | |
| mpx_cld2_1_rev3 62.1 | |
| mpx_cld2_1_rev4 60.8 | |
| mpx_cld2_1_rev5 61.1 | |
| | 106 |
| MPX Clade IIb Reference | caatcgttaagatgtctatcaagatccatgattct |
| NC_055231.1_144914_145019 | caCtcgttaagCtgtctatcaagatccatgattct |
| NC_008291.1_135985_136090 | caCtcgttaagCtgtctatcaagatccatgattct |
| NC_003663.2_152956_153061 | tacaTTtcgttaagCtgtctatcaagatccatgattct |
| NC_006998.1_136036_136141 | acaCCtcgttaagCCtgtctatcaagatccatgattct |
| NC_001611.1_126867_126972 | caCtcgttaagCtgtctatcaagatccatgattct |
| NC_003391.1_138318_138401 | acaCtcgttaagTtgtctatcaagatccatgattct |
| NC_055230.1_151168_151270 | acaCtcgttaagTtgtctatcCagCtccatgattct |
| NC_004105.1_144647_144731 | caCtcAttaagTtgCTtatcCagatccatgattct |
| mpx_cld2_1_fwd1 63.8 | |
| mpx_cld2_1_fwd2 63.3 | |
| mpx_cld2_1_fwd3 62.6 | |
| mpx_cld2_1_fwd4 62.1 | |
| mpx_cld2_1_fwd5 63.7 | |
| mpx_cld2_1_prb1 68.6 | (SEQ ID NO: 60) |
| mpx_cld2_1_prb2 68.3 | (SEQ ID NO: 61) |
| mpx_cld2_1_prb3 67.5 | (SEQ ID NO: 62) |
| mpx_cld2_1_prb4 66.3 | (SEQ ID NO: 63) |
| mpx_cld2_1_rev1 61.9 | atcgttaagatgtctatcaagatcc (SEQ ID NO: 64) |
| mpx_cld2_1_rev2 63.0 | tcgttaagatgtctatcaagatcca (SEQ ID NO: 65) |
| mpx_cld2_1_rev3 62.1 | cgttaagatgtctatcaagatccat (SEQ ID NO: 66) |
| mpx_cld2_1_rev4 60.8 | gttaagatgtctatcaagatccatg (SEQ ID NO: 67) |
| mpx_cld2_1_rev5 61.1 | ttaagatgtctatcaagatccatga (SEQ ID NO: 68) |

MPX Clade IIb Reference (SEQ ID NO: 69); NC_055231.1_144914_145019 (SEQ ID NO: 70); NC_008291.1_135985_136090 (SEQ ID NO: 71); NC_003663.2_152956_153061 (SEQ ID NO: 72); NC_006998.1_136036_136141 (SEQ ID NO: 73); NC_001611.1_126867_126972 (SEQ ID NO: 74); NC_003391.1_138318_138401 (SEQ ID NO: 75); NC_055230.1_151168_151270 (SEQ ID NO: 76); NC_004105.1_144647_144731 (SEQ ID NO: 77).

This analysis uses a small subset of non-MPX OPX genomes (obtained from NCBI). Particularly, the sequences identified as NC_055231.1 (position 144914_14501), NC_008291.1 (position 135985_136090), NC_003663.2 (position 152956_153061), NC_006998.1 (position 136036_136141), NC_001611.1 (position 126867_126972), NC_003391.1 (position 138318_138401), NC_055230.1 (position 151168_151270), and NC_004105.1 (position 144647_144731) are from non-MPX orthopoxvirus genomes. The capitalized and bolded letters are mismatches in the genome sequence compared to the coding region of gene OPG153 of MPXV Clade II (MPX Clade IIb Reference deposited in NCBI Genbank as NC_063383.1). The dashed underlined lines (---) represent a 3nt deletion in the coding region of gene OPG153 of MPXV Clade II compared to the OPX genomes.

The amplification primers would amplify these OPX genomes but the 3nt insertion relative to the probe should prevent probe binding.

Candidate #2-coordinates NC_063383.1_156408-156516
Appears to lie in an intergenic region
Clade II matches-The matches with the_2_nt deletions appear to be all clade IIa

| | |
|---|---|
| | 1 |
| OX297403_156398_156506_1468 | ctacggcatatatacctatttcgtcacttccacacgctccggtaaacgggtgtcatgtgacg |
| OP324469_155995_156103_26 | ctacggcatatatacctatttTgtcacttccacacgctccggtaaacgggtgtcatgtgacg |
| MN346703_157947_158053_17 | ctacgg--tatatacctaCttcgtcacttccacacgctccAgAaaacgggtgtcatgtgacg |
| OK573120_778_672_11 | ctacgg--tatatacctaCttcgtcacttccacacgctccAgtaaacgggtgtcatgtgacg |
| OP215284_156398_156506_2 | ctacggcatatatacctatttcgtcacttccacacgctccggtaaacgggtgtcatgtgacA |
| ON622721_156227_156333_1 | ctacgg--tatatacctatttcgtcacttccacacgctccggtaaacgggtgtcatgtgacg |
| KJ642616_158008_158114_1 | ctacgg--tatatacctaCttcgtcacttcAacacgctccAgtaaacgggtgtcatgtgacg |
| OP215279_156398_156506_1 | ctacggcatatatacctatttTgtcacttccacacgctccggtaaacgggtgtcatgtgacg |
| OP215286_156398_156506_1 | ctacggcatatatacctatttcgtcacttTcacacgctccggtaaacgggtgtcatgtgacg |
| mpx_cld2_2_fwd1 61.6 | tatatacctatttcgtcacttccac (SEQ ID NO: 78) |
| mpx_cld2_2_fwd2 63.3 | atatacctatttcgtcacttccaca (SEQ ID NO: 79) |
| mpx_cld2_2_fwd3 64.0 | tatacctatttcgtcacttccacac (SEQ ID NO: 80) |
| mpx_cld2_2_prb1 71.0 | ctccggtaaacgggtgtcatgtgac |
| mpx_cld2_2_prb2 71.1 | cggtaaacgggtgtcatgtgacg |
| mpx_cld2_2_prb3 69.9 | ggtgtcatgtgacg |
| mpx_cld2_2_prb4 68.2 | gtgtcatgtgacg |
| mpx_cld2_2_prb5 68.2 | tgtcatgtgacg |
| mpx_cld2_2_rev1 61.4 | |
| mpx_cld2_2_rev2 63.1 | |
| mpx_cld2_2_rev3 62.5 | |
| mpx_cld2_2_rev4 61.4 | |
| mpx_cld2_2_rev5 61.2 | |
| | 109 |
| OX297403_156398_156506_1468 | agggagaatcttgataagaggcataatcaatgttgtaatccgatgtc |
| OP324469_155995_156103_26 | agggagaatcttgataagaggcataatcaatgttgtaatccgatgtc |
| MN346703_157947_158053_17 | agggagaatcttgataagaggcataatcaatgttgtaatccgGtgtc |
| OK573120_778_672_11 | agggagaatcttgataagaggcataatcaatgttgtaatccgGtgtc |
| OP215284_156398_156506_2 | agggagaatcttgataagaggcataatcaatgttgtaatccgatgtc |
| ON622721_156227_156333_1 | agggagaatcttgataagaggcataatcaatgttgtaatccgatgtc |
| KJ642616_158008_158114_1 | agggagaatcttgataagaggcataatcaatgttgtaatccgGtgtc |
| OP215279_156398_156506_1 | agggagaatcttgataagaggcataatcaatgttgtaatTcgatgtc |
| OP215286_156398_156506_1 | agggagaatcttgataagaggcataatcaatgttgtaatccgatgtc |
| mpx_cld2_2_fwd1 61.6 | |
| mpx_cld2_2_fwd2 63.3 | |
| mpx_cld2_2_fwd3 64.0 | |
| mpx_cld2_2_prb1 71.0 | (SEQ ID NO: 81) |
| mpx_cld2_2_prb2 71.1 | ag (SEQ ID NO:82) |
| mpx_cld2_2_prb3 69.9 | agggagaatct (SEQ ID NO: 83) |
| mpx_cld2_2_prb4 68.2 | agggagaatctt (SEQ ID NO: 84) |
| mpx_cld2_2_prb5 68.2 | agggagaatcttg (SEQ ID NO : 85) |
| mpx_cld2_2_rev1 61.4 | aagaggcataatcaatgttgtaatc (SEQ ID NO: 86) |
| mpx_cld2_2_rev2 63.1 | agaggcataatcaatgttgtaatcc (SEQ ID NO: 87) |
| mpx_cld2_2_rev3 62.5 | gcataatcaatgttgtaatccgatg (SEQ ID NO: 88) |
| mpx_cld2_2_rev4 61.4 | cataatcaatgttgtaatccgatgt (SEQ ID NO: 89) |
| mpx_cld2_2_rev5 61.2 | ataatcaatgttgtaatccgatgtc (SEQ ID NO: 90) |

OX297403_156398_156506_1468 (SEQ ID NO: 91); OP324469_155995_156103_26 (SEQ ID NO: 92); MN346703_157947_158053_17 (SEQ ID NO: 93); OK573120_778_672_11 (SEQ ID NO: 94); OP215284_156398_156506_2 (SEQ ID NO: 95); ON622721_156227_156333_1 (SEQ ID NO: 96); KJ642616_158008_158114_1 (SEQ ID NO: 97); OP215279_156398_156506_1 (SEQ ID NO: 98); OP215286_156398_156506_1 (SEQ ID NO: 99).

The sequences identified as OX297403 (position 156398_156506), OP324469 (position 155995_156103), MN346703 (position 157947_158053), OK573120 (position 778_672), OP215284 (position 156398_156506), ON622721 (position 156227_156333), KJ642616 (position 158008_158114), OP215279 (position 156398_156506), and OP215286 (position 156398_156506) are from Clade IIa genomes (obtained from NCBI). The capitalized and bolded letters are mismatches in the genome sequences compared to the OPG183 intergenic region of MPXV Clade II (Gene Accession No. (GenBank) NC_063383.1). The sequences identified mpx_cld2_2_fwd, mpx_cld2_2_prb, and mpx_cld2_2_rev represent possible forward primers, probes, and reverse primers that can be used to detect Clade II in a MPX assay. The dashed shaded lines (--) represent a 2nt deletion in the Clade IIa genomes compared to the OPG183 intergenic region of MPXV CladeII.

The above shows no significant matches with Clade I. Therefore, this amplicon lies within a larger segment that is not present in Clade I.

Candidate #2-OPX matches
This analysis uses a small subset of non-MPX OPX genomes

| | |
|---|---|
| | 1 |
| MPX Clade IIb Reference | ctacggcatatatacctatttcgtcacttccacacgctccggtaaacgggtgtcatgtgacg |
| NC_055231.1_168806_168907 | ctacggcaGatatacctaCttcgtcactGccacacgctccggtaaacgg--gtcatgtgacg |
| NC_004105.1_168152_168252 | ctacggcaGatatacctaCttcgtcactGccacacgctccggtaaacgg--gtcatgtgacg |
| NC_003663.2_176827_176927 | -tacAgcaGatatacctaCttcgtcactGccacacgctccggtaaacgg--gtcatgtgacg |
| NC_003391.1_160851_160948 | -tacggcaGatatacctaCtCcgCcac---cacaTgctccggtaaacgg--gtcatgtgCcg |
| NC_008291.1_158375_158472 | -tacggcaGatatacctaCtCcgCcac---cacaTgctccggtaaacgg--gtcatgtACcg |
| NC_006998.1_159720_159777 | --------------------------------------------aacgg--gtcatgtgacA |
| NC_055230.1_174991_175061 | -----------------------------ccacaTCctccTgtaGacAg--AtcatgCAaTg |
| mpx_cld2_2_fwd1 61.6 | tatatacctatttcgtcacttccac (SEQ ID NO: 100) |
| mpx_cld2_2_fwd2 63.3 | atatacctatttcgtcacttccaca (SEQ ID NO: 101) |
| mpx_cld2_2_fwd3 64.0 | tatacctatttcgtcacttccacac (SEQ ID NO: 102) |
| mpx_cld2_2_prb1 71.0 | ctccggtaaacgggtgtcatgtgac |
| mpx_cld2_2_prb2 71.1 | cggtaaacgggtgtcatgtgacg |
| mpx cld2_2_prb3 69.9 | ggtgtcatgtgacg |
| mpx_cld2_2_prb4 68.2 | gtgtcatgtgacg |
| mpx_cld2_2_prb5 68.2 | tgtcatgtgacg |
| mpx_cld2 2_rev1 61.4 | |
| mpx_cld2_2_rev2 63.1 | |
| mpx_cld2_2_rev3 62.5 | |
| mpx_cld2_2_rev4 61.4 | |
| mpx_cld2_2_rev5 61.2 | |
| | 111 |
| MPX Clade IIb Reference | agggaga--atcttgataagaggcataatcaatgttgtaatccgatgtc |
| NC_055231.1_168806_168907 | agggagaatatcttgataagaggTataatcaatgttgtaatc------- |
| NC_004105.1_168152_168252 | agggagaatatcttgataa-aggcataatcaatgttgtaatc------- |
| NC_003663.2 176827_176927 | aCggagaatatcttgataagaCgcataatcaatgttgtaatc------- |
| NC_003391.1_160851_160948 | agggagaatatcttgataagaCTAataatcGatgttgtaatc------- |
| NC_008291.1_158375_158472 | agggagaatatcttgataagaCTcataatTGatgttgtaatc------- |
| NC_006998.1_159720_159777 | agggagaatatcttgataagaggcataatcaatgttgtaatc------- |
| NC_055230.1_174991_175061 | agggagaatatcttgataGAaCgACtaatcaatgttgCaa--------- |
| mpx_cld2_2_fwd1 61.6 | |
| mpx_cld2_2_fwd2 63.3 | |
| mpx_cld2_2_fwd3 64.0 | |
| mpx_cld2_2_prb1 71.0 | (SEQ ID NO: 103) |
| mpx_cld2_2_prb2 71.1 | ag (SEQ ID NO:104) |
| mpx_cld2_2_prb3 69.9 | agggaga-atct (SEQ ID NO: 105) |
| mpx_cld2_2_prb4 68.2 | agggaga-atctt (SEQ ID NO: 106) |
| mpx_cld2_2_prb5 68.2 | agggaga-atcttg (SEQ ID NO: 107) |
| mpx_cld2_2_rev1 61.4 | aagaggcataatcaatgttgtaatc (SEQ ID NO: 108) |
| mpx_cld2_2_rev2 63.1 | agaggcataatcaatgttgtaatcc (SEQ ID NO:109) |
| mpx_cld2_2_rev3 62.5 | gcataatcaatgttgtaatccgatg (SEQ ID NO: 110) |
| mpx_cld2_2_rev4 61.4 | cataatcaatgttgtaatccgatgt (SEQ ID NO: 111) |
| mpx_cld2_2_rev5 61.2 | ataatcaatgttgtaatccgatgtc (SEQ ID NO: 112) |

MPX Clade IIb Reference (SEQ ID NO: 113); NC_055231.1_168806_168907 (SEQ ID NO: 114); NC_004105.1_168152_168252 (SEQ ID NO: 115); NC_003663.2_176827_176927 (SEQ ID NO: 116); NC_003391.1_160851_160948 (SEQ ID NO: 117); NC_008291.1_158375_158472 (SEQ ID NO: 118); NC_006998.1_159720_159777 (SEQ ID NO: 119); NC_055230.1_174991_175061 (SEQ
ID NO: 120).

The sequences identified as NC_055231.1 (position 168806_168907), NC_004105.1 (position 168152_168252), NC_003663.2 (position 176827_176927), NC_003391.1 (position 160851_160948), NC_008291.1 (position 158375_158472), NC_006998.1 (position 159720_159777), and NC_055230.1 (position 174991_175061) are from non-MPX orthopoxvirus genomes. The capitalized and bolded letters are mismatches in the genome sequence compared to the OPG183 intergenic region of MPXV Clade II (MPX Clade IIb Reference: Gene Accession No. (GenBank) NC_063383.1). The underlined, dashed lines (--) represent a 2nt deletion in the OPG183 intergenic region of MPXV Clade II compared to the OPX genomes.

Mismatches in the amplification primers should prevent amplification in this sample of genomes. The two 2nt insertion and deletion relative to the probe should prevent binding.

### Example 2: MPOX in Silico Report for Inclusivity and Exclusivity Analysis for Non-Variola Orthopoxvirus and MPOX Virus (Clade II)

The illustrative MPOX assay described in this example is an automated real-time PCR test for the qualitative detection of DNA from MPOX virus clade II and/or non-variola Orthopoxvirus DNA in lesion swab specimens (i.e., swabs of acute pustular or vesicular rash) collected from individuals suspected of MPOX infection by their healthcare provider.

Testing on the GeneXpert Dx and GeneXpert Infinity instruments include laboratories certified under the Clinical Laboratory Improvement Amendments of 1988 (CLIA), 42 U.S.C. § 263a that meet the regulatory requirements to perform high or moderate complexity testing. Testing on the GeneXpert Xpress (Hub Configuration) instruments is authorized for use at the Point of Care (POC), i.e., in patient care settings operating under a CLIA Certificate of Waiver, Certificate of Compliance, or Certificate of Accreditation.

Results are for the detection and identification of MPOX virus (clade II) and/or non-variola Orthopoxvirus DNA. The MPOX virus (clade II) and/or non-variola Orthopoxvirus DNA is generally detectable in lesion swabs during the acute phase of infection. Positive results are indicative of the presence of MPOX virus (clade II) and/or non-variola Orthopoxvirus DNA: clinical correlation with patient history and other diagnostic information is appropriate to determine patient infection status.

### Principle of the Procedure

The MPOX test is an automated in vitro test for the qualitative detection and identification of MPOX (clade II) viral DNA and non-variola Orthopoxvirus DNA. In some examples, the MPOX test can be used as a diagnostic test. In other examples, the MPOX test is a non-diagnostic test. The MPOX test can be performed on GeneXpertR: Instrument Systems. The GeneXpert Instrument Systems automate and integrate sample preparation, nucleic acid extraction and amplification, and detection of the target sequences in simple or complex samples using real-time PCR assays. The systems include an instrument, computer, and preloaded software for running tests and viewing the results. The systems require the use of single-use disposable cartridges that hold the real-time PCR reagents and host the real-time PCR process. Because the cartridges are self-contained, cross-contamination between samples is minimized. The cartridges can be stored at room temperature or below, such as from 0-30° C. or from 2-28ºC.

The MPOX test includes reagents for the detection of MPXV-clade II and OPXV-E9L NVAR targets in lesion swab specimens. A Sample Processing Control (SPC), a Sample Adequacy Control (SAC), and a Probe Check Control (PCC) are also included in the cartridge utilized by the GeneXpert instrument. The SPC is present to control for adequate processing of the sample and to monitor for the presence of potential inhibitor(s) in the real-time PCR reaction. The SPC also ensures that the real-time PCR reaction conditions (temperature and time) are appropriate for the amplification reaction and that the real-time PCR reagents are functional. The SAC reagents detect the presence of a single copy human gene and monitor whether the sample contains human DNA. The PCC verifies reagent rehydration. PCR tube filling, and confirms that all reaction components are present in the cartridge including monitoring for probe integrity and dye stability.

The exemplary MPOX test is used with lesion swabs collected by a health care provider from patients suspected of having MPOX infection.

The specimen is collected and placed into a transport tube containing 3 mL of viral transport medium (VTM). The specimen is briefly mixed by rapidly inverting the collection tube 5 times. Using the supplied transfer pipette, the sample is transferred to the sample chamber of the Xpert MPOX cartridge. The GeneXpert cartridge is loaded onto the GeneXpert Instrument System platform, which performs hands-off, automated sample processing, and real-time PCR for detection of viral DNA.

The MPOX test targets two genes of the MPOX virus (clade II)-targeting the gene OPG153—Orthopoxvirus A26L/A30L protein, and an intergenic region. In addition, it targets the E9L gene of the Orthopoxvirus, along with primers and probes present for assay internal and external controls, as shown below:
2 targets for MPOX virus (clade II-specific):
1 target for Orthopoxvirus (non-variola):
1 target for Sample Processing Control (SPC); and
1 target for Sample Adequacy Control (SAC).

This example focuses on the inclusivity and exclusivity analysis for non-variola Orthopoxvirus and MPOX virus (clade II).

### Objective

The first objective of this in silico analysis was to estimate the performance of the MPOX test in detection of sets of genomes representing variants of MPOX virus (clade II) and Orthopoxvirus (non-variola) using relevant sequences listed inclusivity disease pathogens. The second objective was to estimate the risk of cross-reactivity of MPOX virus (clade II) and Orthopoxvirus (non-variola) to non-MPOX virus and non-Orthopoxvirus organisms as well as towards all listed exclusivity disease pathogens. The third objective was to analyze Sample Processing Control (SPC) and Sample Adequacy Control (SAC) assay oligos for potential cross-reactivity to non-target sequence. This example covers the in silico inclusivity and exclusivity analysis of the non-variola Orthopoxvirus and MPOX virus (clade II) assay primers and probes.

### Inclusivity Analysis

### Sequence Data Available for In Silico Analysis

GISAID contains a section for MPOX virus (MPXV or MPOX virus) sequences but does not contain other Orthopoxvirus (OPXV) sequences. Most, but not all, of these sequences are annotated with the MPXV clade (I, IIa and IIb) they belong in.

NCBI/GenBank contains sequences for MPXV and other OPXV species. The annotation of sequence records is contributed by the submitter and can vary greatly in detail provided. Significantly, many MPXV sequences do not specify which clade they belong in.

Datasets for MPOX virus were downloaded from NCBI and GISAID. Datasets for cowpox virus, camelpox virus, ectromelia (mousepox) virus and vaccinia virus were downloaded from NCBI. These data sources contain both full length genome sequences and shorter gene segments. For analysis, these shorter segments that may not overlap the amplicons for the assays were removed. The genomes of Orthopoxviruses are on the order of 200,000 nucleotide in length. The arbitrary cutoff of 160,000 nucleotide was chosen as the minimum length for a sequence in the datasets. This eliminates shorter sequences and includes all full-length, or near full-length genomes.

The NCBI datasets were filtered to remove duplicate sequences and those that appear to be synthetic constructs. Duplicate sequences include those included in the RefSeq database, which are duplicates of other GenBank records.

Sequences of vaccinia and cowpox were examined as these viruses, especially vaccinia, can be used as synthetic cloning vectors to introduce foreign genes into mammalian cells. Including these in the analysis dataset may produce misleading inclusivity results as some sequence segments are not part of the virus genome. Furthermore, some sequences are listed in patents. These are also likely to represent synthetic virus constructs and therefore were not included.

The filtered datasets represent virus isolates that have not been subject to other modifications. Results from both datasets are presented separately. A summary of the dataset sources used for each virus is provided in 4.

TABLE 4
Dataset Sources Used for In silico Analysis

| Virus | Source | Filtered Sequences |
|---|---|---|
| MPOX - clade I | GISAID | 43 |
| MPOX - clade IIa | GISAID | 2 |
| MPOX - clade IIb | GISAID | 3,057 |
| MPOX | NCBI | 2,552 |
| Vaccinia | NCBI | 87 |
| Cowpox Vaccinia | NCBI | 9287 |
| CamelpoxCowpox | NCBI | 992 |
| EctromeliaCamelpox | NCBI | 129 |
| Ectromelia | NCBI | 12 |

### Description of MPOX Test Oligos and Amplicons

The MPOX test includes five primer/probe sets for two target regions in the MPXV (Clade II) genome, one target region in the non-variola OPXV genome, a Sample Adequacy Control-human genome (SAC), and a Sample Processing Control (SPC). The forward primer (F), reverse primer (R) and probe (P) sequences are provided in Table 5. Associated amplicon sequences can be found in Table 6.

TABLE 5
Xpert MPOX Assay Oligos

| Organism | Gene | Oligo Sequences | SEQ ID NO: |
|---|---|---|---|
| MPOX virus | OPG153 | GTTCAAGATCCCTGATTCTAGTCTT | 25 |
| (Clade II) | | GGATCTTGATAGACATCTTAACGAT | 26 |
| | | CTCAGAAGATGCTCCGTTACCGTTT | 27 |
| | Intergenic | TATACCTATTTCGTCACTTCCACAC | 29 |
| | region | GGATTACAACATTGATTATGCCTCT | 30 |
| | | AGATTCTCCCTCGTCACATGACACC | 31 |
| Non-Variola | E9L | TCAACTGAAAAGGCCATCTATGA | 33 |
| Orthopoxvirus | | GAGTATAGAGCACTATTTCTAAATCCCA | 34 |
| | | CCATGCAATATACGTACAAGATAGTAGCCAAC | 35 |

TABLE 6
Xpert MPOX Assay Amplicon Sequences

| | | | | SEQ |
|---|---|---|---|---|
| | | | | ID |
| Organism | Gene | Amplicon Sequences | Length | NO: |
| MPOX virus | OPG153 | GTTCAAGATCCCTGATTCTAGTCTTTAGCCTATTTA | 104 | 121 |
| (Clade II) | | CCTCCTCAGAAGATGCTCCGTTACCGTTTTTACAAT | | |
| | | CGTTAAGATGTCTATCAAGATCCATGATTCT | | |
| | Intergenic | CTACGGCATATATACCTATTTCGTCACTTCCACACG | 110 | 122 |
| | region | CTCCGGTAAACGGGTGTCATGTGACGAGGGAGAAT | | |
| | | CTTGATAAGAGGCATAATCAATGTTGTAATCCGAT | | |
| | | GTC | | |
| Non-Variola | E9L | GAGTATAGAGCACTATTTCTAAATCCCATCAGACC | 102 | 36 |
| Orthopoxvirus | | ATATACTGAGTTGGCTACTATCTTGTACGTATATTG | | |
| | | CATGGAATCATAGATGGCCTTTTCAGTTGA | | |

### BLAST Analysis of Xpert MPOX Test Amplicons

Procedures: For the OPX assay and the two MPX assays, the corresponding amplicons were defined in a MPX reference sequence (GenBank NC_063383). These were compared to all the available sequences using BLASTN. Matching segments were extracted, condensed into a set of unique sequences, and aligned.

In a small number of instances, the region that should match the amplicon contains an extended run of ‘N’ characters due to sequencing/assembly errors. These were excluded from the analysis.

Some of the sequences contain runs of ‘N’ characters which represent unknown bases in these positions. Characters other than A, C, G or T represent ambiguity codes. To determine the inclusivity of each oligo, the aligned sequences were first filtered to remove those that contain ambiguous or unknown nucleotides. The number of mismatches to each oligo were counted in this unambiguous dataset.

Table 7, Table 8, and Table 9 show the percentage of matches with 0, 1 or more than 1 mismatch, along with the actual counts in each category. In general, we find that a single mismatch within an oligonucleotide match does not impact primer or probe binding. In many cases an oligo with 2 mismatches may also bind, but in this analysis, we define the percentage inclusivity as the number of oligos that have 0 or 1 mismatch.

### Results

Non-Variola Orthopoxvirus Assay Inclusivity: The predicted inclusivity for the Orthopoxvirus assay probe is 98.9%, reverse primer is 99.8% and forward primer is 100% (see Table 7). Sequences with two or more mismatches are from vaccinia, cowpox and camelpox isolates. Not all the isolates from these species contain the mismatches. Two MPX sequences (ncbi_MPX_FV537352.1_142088_142183_2) contain multiple mismatches across the amplicon, relative to other MPX amplicons. These are indicative of sequencing/assembly errors.

TABLE 7
Inclusivity Analysis for OPXV Assay

| | | 1 | 2 or more | 0 or 1 mm |
|---|---|---|---|---|
| Oligonucleotide | Exact match | mismatch | mismatches | inclusivity |
| OPX_CDC_E9LF | 99.2% | 0.8% | 0.0% | 100.00% |
| forward primer | (5770/5816) | (44/5816) | (2/5816) | |
| OPX_CDC_E9LP2 | 98.6% | 0.3% | 1.1% | 98.90% |
| probe | (5736/5816) | (16/5816) | (64/5816) | |
| OPX_CDC_E9LR2 | 99.5% | 0.3% | 0.2% | 99.80% |
| reverse primer | (5787/5816) | (19/5816) | (10/5816) | |

MPOX virus (clade II) Assay #1 Inclusivity: The predicted inclusivity for the MPOX virus assay primers and probes can be found in Table 8. This assay is predicted to have 100% inclusivity to both the NCBI and GISAID MPX clade IIa and IIb sequences. The amplicon alignments can be found in Scheme 2. This assay is designed to not detect MPX Clade I isolates. No significant matches to the 43 sequences in the Clade I dataset were found.

TABLE 8
Inclusivity Analysis for MPXV Assay #1

| | | 1 | 2 or more | 0 or 1 mm |
|---|---|---|---|---|
| Oligonucleotide | Exact match | mismatch | mismatches | inclusivity |
| MPX_CII_S1_F1 | 100.0% | 0.0% | 0.0% | 100% |
| forward primer | (5531/5531) | (0/5531) | (0/5531) | |
| MPX_CII_S1_P1_probe | 99.7% | 0.3% | 0.0% | 100% |
| | (5512/5531) | (19/5531) | (0/5531) | |
| MPX_CII_S1_R1 | 100.0% | 0.0% | 0.0% | 100% |
| reverse primer | (5531/5531) | (0/5531) | (0/5531) | |

MPOX virus (clade II) Assay #2 Inclusivity: This assay is predicted to have 100% inclusivity to both the NCBI and GISAID MPX Clade IIa and IIb sequences. The predicted inclusivity for the MPOX virus assay primers and probes can be found in Table 9.

TABLE 9
Inclusivity Analysis for MPXV Assay #2

| | | 1 | 2 or more | 0 or 1 mm |
|---|---|---|---|---|
| Oligonucleotide | Exact match | mismatch | mismatches | inclusivity |
| MPX_C2_S2_F3N | 98.0% | 2.0% | 0.0% | 100% |
| forward primer | (5326/5433) | (106/5433) | (1/5433) | |
| MPX_CII_S2_P3_asCF3 | 99.9% | 0.1% | 0.0% | 100% |
| probe | (5429/5433) | (4/5433) | (0/5433) | |
| MPX_CII_S2_R2reverse | 100.0% | 0.0% | 0.0% | 100% |
| primer | (5431/5433) | (2/5433) | (0/5433) | |

### Conclusion

The MPXV assays #1 and #2 are predicted to have 100% inclusivity to both the NCBI and GISAID MPXV Clade IIa and IIb sequences. These assays are designed to not detect MPXV Clade I isolates. The Non-variola OPXV assay probe has a 98.9% inclusivity, while reverse primer 99.8% and forward primer 100%. The analytical sensitivity of the MPOX target is 50 copies/mL or less or 30 copies/mL or less. The analytical sensitivity of the OPX target is 100 copies/mL or less.

### Exclusivity Analysis

Objective: To estimate the risk of cross reactivity of Xpert MPOX oligos and amplicons to other non-MPOX viruses as well as to all exclusivity disease pathogens.

### BLAST Analysis of Xpert MPOX Test Amplicons

Procedures: For each of the 33 species listed in the EUA submission template, a representative genome was identified in the NCBI RefSeq or Nucleotide databases, the sequences were downloaded and formatted into a BLAST database. All oligonucleotides and amplicons were compared to the dataset using BLAST and the percent homology/identity for each oligo was determined.

### Results

The tables (see Table 10, Table 11, Table 12, Table 13, and Table 14) show 80% identity or more to each of the listed species. The species with a best match of less than 80% identity are left blank. The interpretation of these results is given in the text after each table.

NOTE: Streptococcus Group C and G species are not well characterized in terms of genome sequencing. In this analysis, the genome of Streptococcus dysgalactiae subsp. equisimilis was used to represent both these groups, consistent with the literature.

In the case of matches to the human genome, the specific chromosome that contains the match is listed.

NOTE: Four eukaryotic species contain multiple chromosomes. The sequences for all these were downloaded and searched.

TABLE 10
Exclusivity in silico Analysis Showing Percent Homology of Primers and
Probes for Non-variola OPX Assay with Percent Homology to Assay Oligos

| | Refseq | Forward | | Reverse |
|---|---|---|---|---|
| Species | Accession ID | Primer | Probe | Primer |
| Variola Virus (smallpox) | NC_001611.1 | 100% | 91% | 100% |
| | | (23/23 nt) | (29/32 nt) | (28/28 nt) |
| Herpes simplex virus (HSV-1) | NC_001806 | | | |
| Herpes simplex virus (HSV-2) | LS480640.1 | | | |
| Varicella-zoster virus (Chickenpox) | NC_001348.1 | | | |
| Staphylococcus aureus | AP017922.1 | | | |
| Streptococcus pyogenes | NZ_LS483338.1 | | | |
| Pseudomonas aeruginosa | NC_002516.2 | | | |
| Corynebacterium jeikeium | NC_007164.1 | | | |
| Escherichia coli | NZ_CP077969.1 | | | |
| Bacteroides fragilis | NZ_CP069563.1 | | | |
| Neisseria gonorrhoeae | NZ_CP097846.1 | | | |
| Mycoplasma pneumoniae | NZ_CP014267.1 | | | |
| Human Papilloma virus (HPV) | NC_027779.1 | | | |
| Treponema pallidum | NC_016842.1 | | | |
| molluscum contagiosum virus | MN931752.1 | | | |
| Streptococcus mitis | JVJJ01000007.1 | | | |
| Staphylococcus epidermidis | JUKL01000001.1 | | | |
| Streptococcus agalactiae | NZ_CP012480.1 | | | |
| Lactobacillus species (acidophilus) | NZ_CP054559.1 | | | |
| Acinetobacter calcoaceticus | NZ_CP020000.1 | | | |
| Enterococcus faecalis | NC_021023.1 | | | |
| Streptococcus Group C and Group G* | AP012976.1 | | | |
| Corynebacterium diphtheriae | NZ_CP025209.1 | | | |
| Clamydia trachomatis | NC_000117.1 | | | |
| Mycoplasma genitalium | NC_000908.2 | | | |
| Human Genome | | 83% | | |
| | | (19/23 nt) | | |
| | | chr 8 | | |
| Candida albicans | | | | |
| Trichophyton rubrum | | | | |
| Trichomonas vaginalis | | | | |
| Cowpox virus | NC_003663 | 100% | 100% | 100% |
| | | (23/23 nt) | (32/32 nt) | (28/28 nt) |
| Camelpox virus | NC_003391 | 100% | 94% | 100% |
| | | (23/23 nt) | (30/32 nt) | (28/28 nt) |
| Vaccinia virus | NC_006998 | 100% | 100% | 100% |
| | | (23/23 nt) | (32/32 nt) | (28/28 nt) |
| Ectromelia virus (mousepox) | NC_004105 | 100% | 100% | 96% |
| | | (23/23 nt) | (32/32 nt) | (27/28 nt) |

*see note in para.[0307]. Results

The single match of the forward primer to the human genome cannot produce an amplification product.

The OPX oligonucleotides are based on the design from the CDC targeting the E9L gene. This is described in the paper by Yu Li, et al. (Journal of Clinical Virology 36 (2006) 194-203) pubmed.ncbi.nlm.nih.gov/16731033/. The OPX assay was designed to detect MPOX (clade I and II), Cowpox, Camelpox, Ectromelia and Vaccinia. It was specifically designed to not detect variola and the paper demonstrates that this is the case in laboratory tests. From the in-silico analysis shown in the above table, the forward and reverse primers are exact matches to the Variola genome, and the probe has three mismatches. Even though the probe has 91% identity to the Variola genome, the paper demonstrates that this does not produce a positive assay result in the lab when tested against a wide range of Variola samples (see Table 2 in Yu Li, et al. (Journal of Clinical Virology 36 (2006) 194-203)).

For detailed results and alignments to the target Orthopoxvirus species, refer to the OPX assay section on the inclusivity analysis.

TABLE 11
Exclusivity Analysis for MPXV Assay #1 with Percent Homology to Assay Oligos

| | Refseq | Forward | | Reverse |
|---|---|---|---|---|
| | Accession ID | Primer | Probe | Primer |
| Species | | | | |
| Variola Virus (smallpox) | NC_001611.1 | 88% | 92% | 92% |
| | | (22/25 nt) | (23/25 nt) | (23/25 nt) |
| Herpes simplex virus (HSV-1) | NC_001806 | | | |
| Herpes simplex virus (HSV-2) | LS480640.1 | | | |
| Varicella-zoster virus (Chickenpox) | NC_001348.1 | | | |
| Staphylococcus aureus | AP017922.1 | | | |
| Streptococcus pyogenes | NZ_LS483338.1 | | | |
| Pseudomonas aeruginosa | NC_002516.2 | | | |
| Corynebacterium jeikeium | NC_007164.1 | | | |
| Escherichia coli | NZ_CP077969.1 | | | |
| Bacteroides fragilis | NZ_CP069563.1 | | | |
| Neisseria gonorrhoeae | NZ_CP097846.1 | | | |
| Mycoplasma pneumoniae | NZ_CP014267.1 | | | |
| Human Papilloma virus (HPV) | NC_027779.1 | | | |
| Treponema pallidum | NC_016842.1 | | | |
| molluscum contagiosum virus | MN931752.1 | | | |
| Streptococcus mitis | JVJJ01000007.1 | | | |
| Staphylococcus epidermidis | JUKL01000001.1 | | | |
| Streptococcus agalactiae | NZ_CP012480.1 | | | |
| Lactobacillus species (acidophilus) | NZ_CP054559.1 | | | |
| Acinetobacter calcoaceticus | NZ_CP020000.1 | | | |
| Enterococcus faecalis | NC_021023.1 | | | |
| Streptococcus Group C and Group G* | AP012976.1 | | | |
| Corynebacterium diphtheriae | NZ_CP025209.1 | | | |
| Clamydia trachomatis | NC_000117.1 | | | |
| Mycoplasma genitalium | NC_000908.2 | | | |
| Species with multiple chromosomes* | | | | |
| Human Genome | | 88% | | |
| | | (22/25 nt) | | |
| | | chr 12 | | |
| Candida albicans | | | | |
| Trichophyton rubrum | | | | |
| Trichomonas vaginalis | | | | |
| Cowpox virus | NC_003663 | 100% | 88% | 92% |
| | | (25/25 nt) | (22/25 nt) | (23/25 nt) |
| Camelpox virus | NC_003391 | | 92% | 92% |
| | | | (23/25 nt) | (23/25 nt) |
| Vaccinia virus | NC_006998 | 92% | 92% | 92% |
| | | (23/25 nt) | (23/25 nt) | (23/25 nt) |
| Ectromelia virus (mousepox) | NC_004105 | | 88% | |
| | | | (22/25 nt) | |

*see note in para.[0307]. Results

TABLE 12
Exclusivity Analysis for MPXV Assay #2 with Percent Homology to Assay Oligos

| | | | |
|---|---|---|---|
| Refseq | Forward | | Reverse |
| Accession ID | Primer | Probe | Primer |

| | | | |
|---|---|---|---|
| Species | | | |
| Variola Virus (smallpox) | NC_001611.1 | 84% | |
| | | (21/25 nt) | |
| Herpes simplex virus (HSV-1) | NC_001806 | | |
| Herpes simplex virus (HSV-2) | LS480640.1 | | |
| Varicella-zoster virus (Chickenpox) | NC_001348.1 | | |
| Staphylococcus aureus | AP017922.1 | | |
| Streptococcus pyogenes | NZ_LS483338.1 | | |
| Pseudomonas aeruginosa | NC_002516.2 | | |
| Corynebacterium jeikeium | NC_007164.1 | | |
| Escherichia coli | NZ_CP077969.1 | | |
| Bacteroides fragilis | NZ_CP069563.1 | | |
| Neisseria gonorrhoeae | NZ_CP097846.1 | | |
| Mycoplasma pneumoniae | NZ_CP014267.1 | | |
| Human Papilloma virus (HPV) | NC_027779.1 | | |
| Treponema pallidum | NC_016842.1 | | |
| molluscum contagiosum virus | MN931752.1 | | |
| Streptococcus mitis | JVJJ01000007.1 | | |
| Staphylococcus epidermidis | JUKL01000001.1 | | |
| Streptococcus agalactiae | NZ_CP012480.1 | | |
| Lactobacillus species (acidophilus) | NZ_CP054559.1 | | |
| Acinetobacter calcoaceticus | NZ_CP020000.1 | | |
| Enterococcus faecalis | NC_021023.1 | | |
| Streptococcus Group C and Group G* | AP012976.1 | | |
| Corynebacterium diphtheriae | NZ_CP025209.1 | | |
| Clamydia trachomatis | NC_000117.1 | | |
| Mycoplasma genitalium | NC_000908.2 | | |
| Species with multiple chromosomes* | | | |
| Human Genome | | 84% | 84% |
| | | (21/25 nt) | (21/25 nt) |
| | | Chr 18 | Chr 5 |
| | | 84% | 80% |
| | | (21/25 nt) | (20/25 nt) |
| | | Chr 6 | Chr 10 |
| | | 80% | |
| | | (20/25 nt) | |
| | | Chr 13 | |
| Candida albicans | | | |
| Trichophyton rubrum | | | |
| Trichomonas vaginalis | | | 88% |
| | | | (22/25 nt) |
| Cowpox virus | NC_003663 | 88% | 92% |
| | | (22/25 nt) | (23/25 nt) |
| Camelpox virus | NC_003391 | | 88% |
| | | | (22/25 nt) |
| Vaccinia virus | NC_006998 | | 96% |
| | | | (24/25 nt) |
| Ectromelia virus (mousepox) | NC_004105 | 88% | 88% |
| | | (22/25 nt) | (22/25 nt) |

*see note in para.[0307]. Results

The single match of the forward primer to variola (see Table 12) cannot produce an amplification product. The matches of the forward and reverse primers on the human genome (see Table 12) are located on different chromosomes and therefore cannot produce an amplification product. The <100% match of the reverse primer to Trichomonas vaginalis (see Table 12) cannot produce an amplification product.

The matches of the oligos to the Orthopoxvirus species genomes (see Table 12) could potentially produce an amplification product with Cowpox and Ectromelia genomes. There is a 2 nt insertion in the probe, relative to the OPX genomes and a second 2 nt deletion relative to the genomes. These are sufficient to prevent probe binding with these genomes.

Therefore, the MPX Clade II assay #2 will not detect other Orthopoxvirus genomes.

This assay is designed to not detect MPX Clade I isolates. The primers and probes did not show any concerning homology with Clade I sequences. The best scoring alignment between the MPOX (Clade II) assay #2 amplicon and the MPX Clade I dataset is shown here (one example of 43 identical search results). This region is not aligned with MPOX (Clade II) assay #2 primers and probes, ensuring exclusivity of MPXV Clade I isolates.

TABLE 13
Exclusivity Analysis for Sample Adequacy Control
(SAC) Assay with Percent Homology to Assay Oligos

| | Refseq | Forward | | Reverse |
|---|---|---|---|---|
| | Accession ID | Primer | Probe | Primer |
| Species | | | | |
| Variola Virus (smallpox) | NC_001611.1 | | | |
| Herpes simplex virus (HSV-1) | NC_001806 | | | |
| Herpes simplex virus (HSV-2) | LS480640.1 | | | |
| Varicella-zoster virus (Chickenpox) | NC_001348.1 | | | |
| Staphylococcus aureus | AP017922.1 | | | |
| Streptococcus pyogenes | NZ_LS483338.1 | | | |
| Pseudomonas aeruginosa | NC_002516.2 | | | |
| Corynebacterium jeikeium | NC_007164.1 | | | |
| Escherichia coli | NZ_CP077969.1 | | | |
| Bacteroides fragilis | NZ_CP069563.1 | | | |
| Neisseria gonorrhoeae | NZ_CP097846.1 | | | |
| Mycoplasma pneumoniae | NZ_CP014267.1 | | | |
| Human Papilloma virus (HPV) | NC_027779.1 | | | |
| Treponema pallidum | NC_016842.1 | | | |
| molluscum contagiosum virus | MN931752.1 | | | |
| Streptococcus mitis | JVJJ01000007.1 | | | |
| Staphylococcus epidermidis | JUKL01000001.1 | | | |
| Streptococcus agalactiae | NZ_CP012480.1 | | | |
| Lactobacillus species (acidophilus) | NZ_CP054559.1 | | | |
| Acinetobacter calcoaceticus | NZ_CP020000.1 | | | |
| Enterococcus faecalis | NC_021023.1 | | | |
| Streptococcus Group C and Group G* | AP012976.1 | | | |
| Corynebacterium diphtheriae | NZ_CP025209.1 | | | |
| Clamydia trachomatis | NC_000117.1 | | | |
| Mycoplasma genitalium | NC_000908.2 | | | |
| Species with multiple chromosomes* | | | | |
| Human Genome | | 100% | 100% | 100% |
| | | (23/23 nt) | (23/23 nt) | (23/23 nt) |
| | | Chr 11 | Chr 11 | Chr 11 |
| | | 87% | 87% | 86% |
| | | (20/23 nt) | (20/23 nt) | (19/22 nt) |
| | | Chr 11 | Chr X | Chr 21 |
| | | 87% | 87% | 82% |
| | | (20/23 nt) | (20/23 nt) | (18/22 nt) |
| | | Chr 12 | Chr 19 | Chr 8 |
| | | 87% | | 82% |
| | | (20/23 nt) | | (18/22 nt) |
| | | Chr 2 | | Chr 2 |
| | | 87% | | |
| | | (20/23 nt) | | |
| | | Chr 1 | | |
| | | 83% | | |
| | | (19/23 nt) | | |
| | | Chr 2 | | |
| | | 83% | | |
| | | (19/23 nt) | | |
| | | Chr 1 | | |
| | | 83% | | |
| | | (19/23 nt) | | |
| | | Chr 3 | | |
| | | 83% | | |
| | | (19/23 nt) | | |
| | | Chr X | | |
| | | 83% | | |
| | | (19/23 nt) | | |
| | | Chr 20 | | |
| | | 83% | | |
| | | (19/23 nt) | | |
| | | Chr 17 | | |
| | | 83% | | |
| | | (19/23 nt) | | |
| | | Chr 19 | | |
| | | 83% | | |
| | | (19/23 nt) | | |
| | | Chr 13 | | |
| | | 83% | | |
| | | (19/23 nt) | | |
| | | Chr 9 | | |
| Candida albicans | | 83% | | |
| | | (19/23 nt) | | |
| Trichophyton rubrum | | | | |
| Trichomonas vaginalis | | | | |
| Cowpox virus | NC_003663 | | | |
| Camelpox virus | NC_003391 | | | |
| Vaccinia virus | NC_006998 | | | |
| Ectromelia virus (mousepox) | NC_004105 | | | |

*see note in para.[0307]. Results

The Sample Adequacy Control (SAC) oligonucleotides amplify a segment of the human Hydroxymethylbilane Synthase gene (HMBS). These oligonucleotides are a 100% match in the human genome, as shown above (see Table 13). The <100% match to other human genome chromosomes (see Table 13) were examined and none of them are located close enough to each other for the formation of an amplification product. Therefore, none of these matches pose a problem to the assay. The <100% match of the forward primer to Candida albicans (see Table 13) does not pose a problem to the assay, as the assay reverse primer and probe homology is less than 80%.

TABLE 14
Exclusivity Analysis for Sample Preparation Control
(SPC) Assay with Percent Homology to Assay Oligos

| | | | |
|---|---|---|---|
| Refseq | Forward | | Reverse |
| Accession ID | Primer | Probe | Primer |

| | | | |
|---|---|---|---|
| Species | | | |
| Variola Virus (smallpox) | NC_001611.1 | | |
| Herpes simplex virus (HSV-1) | NC_001806 | | |
| Herpes simplex virus (HSV-2) | LS480640.1 | | |
| Varicella-zoster virus (Chickenpox) | NC_001348.1 | | |
| Staphylococcus aureus | AP017922.1 | | |
| Streptococcus pyogenes | NZ_LS483338.1 | | |
| Pseudomonas aeruginosa | NC_002516.2 | | |
| Corynebacterium jeikeium | NC_007164.1 | | |
| Escherichia coli | NZ_CP077969.1 | | |
| Bacteroides fragilis | NZ_CP069563.1 | | |
| Neisseria gonorrhoeae | NZ_CP097846.1 | | |
| Mycoplasma pneumoniae | NZ_CP014267.1 | | |
| Human Papilloma virus (HPV) | NC_027779.1 | | |
| Treponema pallidum | NC_016842.1 | | |
| molluscum co ntagiosum virus | MN931752.1 | | |
| Streptococcus mitis | JVJJ01000007.1 | | |
| Staphylococcus epidermidis | JUKL01000001.1 | | |
| Streptococcus agalactiae | NZ_CP012480.1 | | |
| Lactobacillus species (acidophilus) | NZ_CP054559.1 | | |
| Acinetobacter calcoaceticus | NZ_CP020000.1 | | |
| Enterococcus faecalis | NC_021023.1 | | |
| Streptococcus Group C and Group G* | AP012976.1 | | |
| Corynebacterium diphtheriae | NZ_CP025209.1 | | |
| Clamydia trachomatis | NC_000117.1 | | |
| Mycoplasma genitalium | NC_000908.2 | | |
| Species with multiple chromosomes* | | | |
| Human Genome | | 83% | 81% |
| | | (19/23 nt) | (22/27 nt) |
| | | Chr4 | Chr13 |
| Candida albicans | | | |
| Trichophyton rubrum | | | |
| Trichomonas vaginalis | | | |
| Cowpox virus | NC_003663 | | |
| Camelpox virus | NC_003391 | | |
| Vaccinia virus | NC_006998 | | |
| Ectromelia virus (mousepox) | NC_004105 | | |

*see noe in para.[0307]. Results

The two oligo matches to the human genome (see Table 14) are located on different chromosomes and therefore cannot produce an amplification product.

Cross Reactivity Between Assay Oligonucleotides: The MPOX assay is a multiplex of five component assays, each of which uses two amplification primers and a probe. The full set of 15 oligonucleotides were compared to each other to check for potential inter-oligo binding. No significant homology was identified with any pair of oligos in the complete set.

### Conclusions

The MPOX test meets the inclusivity and exclusivity requirements. In-silico cross reactivity check did not show any concerning interactions between the test primers and probes.

### Example 3: MPOX Assay of Samples with Varying Concentrations of Inactivated MPOX Virus

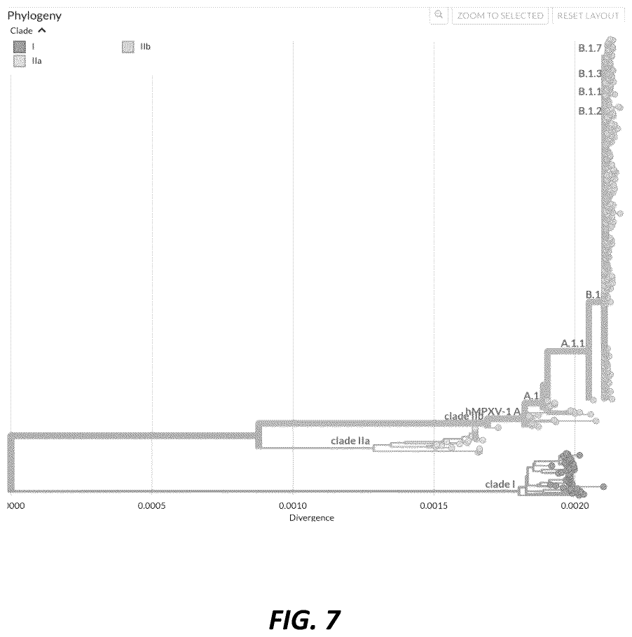

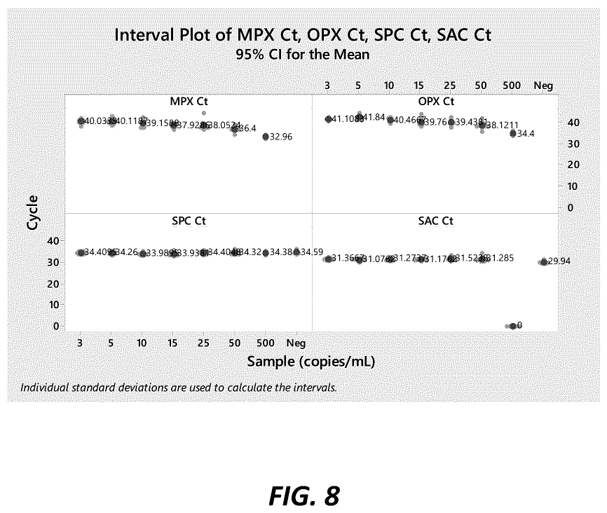

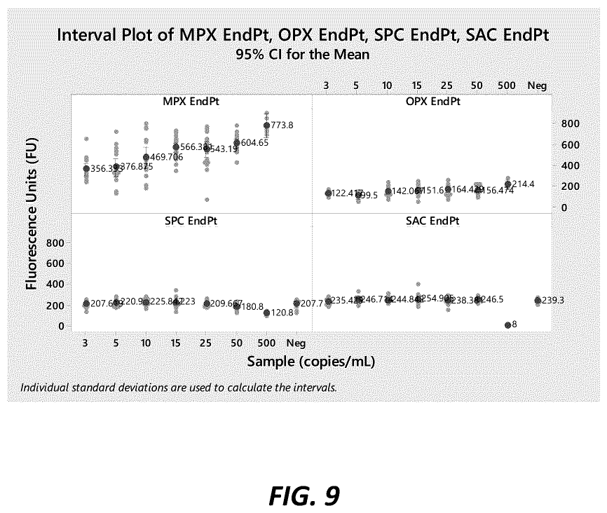

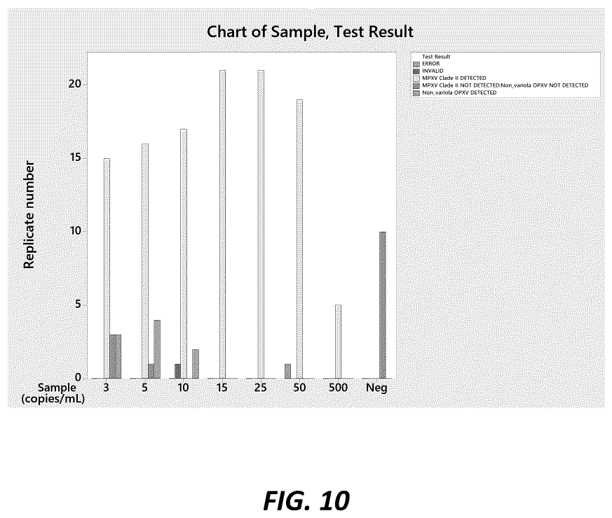

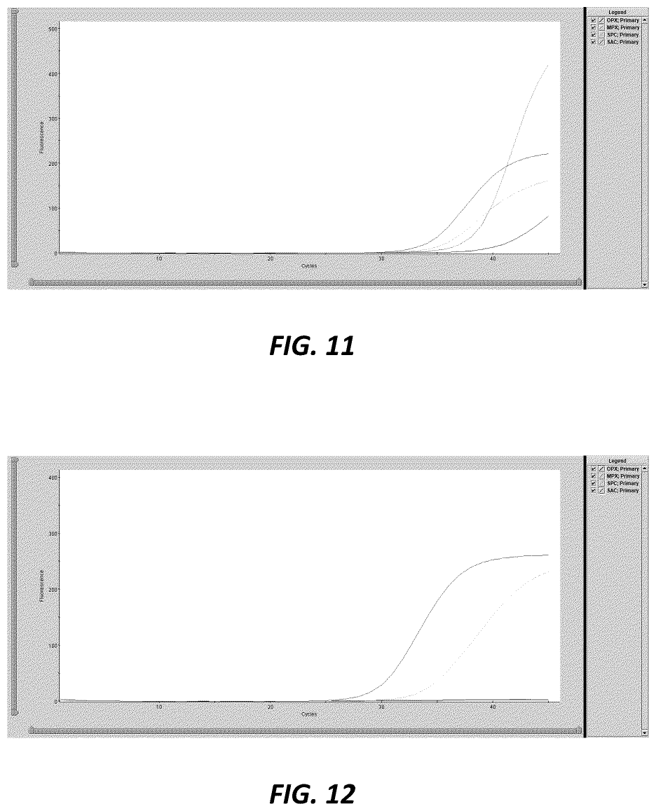

The MPOX panel assay was carried out using the GENEXPERT® system essentially as described above. Positive samples contained inactivated MPOX virus with final concentrations varying from 3 to 500 copies/mL, human cells and SPC template. Negative samples included human cells and SPC template only. FIG. 8 shows the Ct values of each analyte. FIG. 9 shows the end point fluorescence (EPF) for each analyte. FIG. 10 shows the test results from the GENEXPERT® software. FIG. 11 shows a fluorescence vs. cycles output graph for 50 copies/mL NATtrol MPOX Virus input. FIG. 12 shows a fluorescence vs. cycles output graph for a negative sample. The results show that the assay performs as expected.

SEQUENCES

SEQ ID NO: 1: MPX Clade II OPG153 Gene

| | |
|---|---|
| 132361 | .......... .......... .......... .....ttaaa ccgtcttttc tggtataata |
| 132421 | ttgtttagta gatactcatc aagataagct aattcactaa acatattatc ggattcggta |
| 132481 | ttgttactcg agaatagagt tcgttatgct cctgatattc ggaaatctgt ggagtttcag |
| 132541 | gttttggtgg aagtgtaact gctacttggt gggatactga aggatatttc agagagttgt |
| 132601 | ggatgttcgg gttcgacatc caccgatggt gtcacgccac taatcggttc ggtaacgtct |
| 132661 | gtggatggag gtgctacttc tacagaacct gtagcctcag ttgtcaacgg agatacatat |
| 132721 | tcaatgcgcg gaaatgtata atttggtaat ggtttctcat gtggatctta agaagaagag |
| 132781 | gtaagatatc tacgaaagat accgatcacg tttctagttc tcttttgtag aactttaact |
| 132841 | ttttctttct cagcatctag ttgatattcc gacctcttca cgtttcgcat gggttacctc |
| 132901 | cgcagttttt acaagcgatt tcacgttcca gatcacgttc agccttcata cgtctctccc |
| 132961 | tctctctatc gagtttatca gagcagtctt tctgaaggcg atcgaactcc ataaatttct |
| 133021 | ccaacgcttt gattgtttcc atagatttcc gaagtttagc ttctaggacg gcgattcttt |
| 133081 | tttttttttt tttttttttt ttcgaattca cggggtacaa ccgtttccat taccaccatc |
| 133141 | tctatgtttc ttttctagat cggcaatctt tctcaatctt tctcaacatt tcatccccat |
| 133201 | accttttcat tcctcgagtc tattgtcgtc gaaatatcgt tccagctcct tttcgacctc |
| 133261 | aataacttta gcacgttgtt tcatcaagct ctctcttgta gtactatcat ttttatctga |
| 133321 | ttccctgaca cgtttaagat cttcatgtaa ttgagtcagc tcttgacgca atctcttaac |
| 133381 | taacttcctc tcttgcttct tcgtcatagt acttacaatc actatgggat ccattgttac |
| 133441 | cacgtctgta ctcgacgagc tcacgtttaa gagattcaat ttccagtttg tatcggtcca |
| 133501 | tgtctccatt gctacaccac cattagattt acaggctgct agttgtcgtt cgagatcaga |
| 133561 | aatacgtgtt ttcttggaat ggatttcgtc gatgtacttg tcatgattgg catcgaaaca |
| 133621 | cttattaagt tctttttttc aattctacga ttttatttct ttcgcgagtc aattccctcc |
| 133681 | tgtagtaact atcagttttg tcagattcac gctctctacg tagactttct tgtaagttac |
| 133741 | taatttgttc cctggcatta ccgagttcag ttttatatgc cgaatagagt tctgattcat |
| 133801 | cctttgagaa gatctctagc gatcgttcaa gatccctgat tctagtcttt agcctattta |
| 133861 | cctcctcaga agatgctccg ttaccgtttt tacaatcgtt aagatgtcta tcaagatcca |
| 133921 | tgattctatc tcttttccat atcagcattg atttcattat tacgttcgca gtcgttcaac |
| 133981 | tgtatttcaa gatctgagat tctagattgt aatctctgta gcatttccac ggcattcact |
| 134041 | cagttgtctt tcaagatctg agattctaga ttggagtctg ctaatctctg taagatttcc |
| 134101 | tcctccgctc tcgatgcagt cggtcaactt attctctagt tctctaatac gcgaacgcag |
| 134161 | tgcatcaact tcttgtgtgt cttcttgatt gcgtgtgcat tcatcgagtc tagattcgag |
| 134221 | atctctaacg tgacgtcgtt cttcctcaag ttctctgtgt actacagaaa gcgtgtccct |
| 134281 | atcttgttga tatttagcaa tttctgattc tagagtactg attctactca cgtatgtact |
| 134341 | aatagttgtc ttagccttat caagatcctc cttgtatttg tcacattcct tgatatccat |
| 134401 | acgaagtctg gacagttccc attcgacatt acgacgttta tcgatttcag ctcggagatc |
| 134461 | gtcgtcgcgt tgttttagcc acatacgact aagttcaagt tctcgttgac aagatccatc |
| 134521 | tacttttcca tccctaatag tatccagttc cttttctagt tctgaccgca tttctcgttc |
| 134581 | catatcaaga gattctctca attctcgtat agtcttctta tcaatttctg atgaatctga |
| 134641 | accatcatct gtcccatttt gttgcatatc cctgagttct ttgatctctg ttgtaagtct |
| 134701 | gtcgattctt tcggttttat aaacagaatc cctttccaaa gtcctaatct tactgagttt |
| 134761 | atcattaagt tcttcattca attcagtgag ttttctcttg gcttcttcca agtctgtttt |
| 134821 | aaactctcca tcatttccgc attcttcctc gcatttatct aaccattcaa ttagtttatt |
| 134881 | aataactagt tggtaatcag cgattcctat agccgttctt gtatttgtgg gaacataatt |
| 134941 | aggatcttct aatggattgt atggcttgat agcatcatct ttatcattat taggtggggg |
| 135001 | atggacaacc ttaattggtt ggtcctcctt atctcctcca gtagcatgtg gttcttcaat |
| 135061 | accagtatta gtaataggct tagacaaatg cttgtcgtac gcgggcactt cctcatccat |
| 135121 | caagtattta taatcgggtt ctgtttcaga atattctttt ctaagagacg cgacttcagg |
| 135181 | agttagtaga agaactctgt ttctgtatct atcaacgctg gaatcgatac tcaagttaag |
| 135241 | gatagcgaat acctcatcgt catcatccgt atcttctgaa acgccatcat atgacatttc |
| 135301 | atgaagtcta acgtattgat aaacagaatc agatttagta ttaaacagat ccttgacctt |
| 135361 | tttagtaaat gcatatgtat attttagatc tccagatttc ataatatgat cgcatgcctt |
| 135421 | aaatgtcaat gcttccatga tatagtctgg aacactaatg ggtgacgaaa aagatacagc |
| 135481 | accatatgct acgttgataa atagatctga accactaagt agataatgat taatgttaag |
| 135541 | gaagaggaaa tattcagtat atagatatgc cttagcatca tatcttgtac taaacacgct |
| 135601 | aaacagttta ttgatgtgat caatttccaa cagaacaatt agagcggcag gaataccaac |
| 135661 | aaacatatta ccacatccgt attttctatg aatatcacat atcatattaa aaaatcttga |
| 135721 | tagaagagcg aatatctcgt ctgacttaat gagatgtagt tcagcagcat aagtcataac |
| 135781 | tgtaaataga acatactttc ctgtagtgtt gattctagac tccacatcaa caccattatt |
| 135841 | aaaaatagtt ttatatacat ctttaatctg ctctccgtta atcgtcgaac gttctagtat |
| 135901 | acggaaacac tttgatttct tatctgtagt taatgactta gtgatatcac gaagaatatt |
| 135961 | acgaattaca tttcttgttt ttcttgagag acctgattca gaactcaact catcgttcca |
| 136021 | tagtttttct acctcagtgg cgaaatcttt ggagtgtttg gtacattttt taataaggtt |
| 136081 | cgtgacctcc at........ .......... .......... .......... .......... |

SEQ ID NO: 2: MPX Clade II OPG183 Intergenic Region (Gene Accession No.
(GenBank) NC_063383.1)

| | |
|---|---|
| 156301 | .......... .......... .......... .......... .......... ....atggat |
| 156361 | ataaagaatt tactgactac atgtactatt ttacattact acattggcta cggcatatat |
| 156421 | acctatttcg tcacttccac acgctccggt aaacgggtgt catgtgacga gggagaatct |
| 156481 | tgataagagg cataatcaat gttgtaatcc gatgtccacc tggagaattt gccaaggtca |
| 156541 | gatgtagagt tggtagtgat aacacaaaat gtgaacactg cccacctcat acatataccg |
| 156601 | caatccccaa ttattctaat agatgtcatc aatgtagaaa atgcccaaca ggatcatttg |
| 156661 | ataaggtaaa gtgtaccgga acacagaaca aatgttcgtg tcatcctggt tggtatacgc |
| 156721 | tactgattct tcacagactg aagattgtcg agatttgtgt accaaaaaag gagatgtcca |
| 156781 | tgcggatact ttggtggaat agatgaagga aatcctattt gtaaatcgtg ttgtgttggt |
| 156841 | gaatattgcg actacctacg taattataga cttgatccat ttcctccatg caaactatct |
| 156901 | atctaaatgt aattaattat .......... .......... .......... .......... |

SEQ NO: 3: MPX Strain F8L gene (similar to E9L gene)

| | |
|---|---|
| 53641 | .......... .......... .......... .......... .......... ttatgcttcg |
| 53701 | taaaatgtag gtcttgaacc aaacattctt tgaaaaaatg agatgcataa aactttatta |
| 53761 | tccaatagat taactatttc agacgtcaat cgtttaaagt aaacttcgta aaatattctt |
| 53821 | tgattgctgc cgagtttaaa acttctatcg ataattgttt catatgtttt aatatttaca |
| 53881 | agttttttgg tccatggtac attagctgga cagatatatg caaaataata tcgttctcca |
| 53941 | agttctatag tctctggatt gtttttatta tattcagtaa ccaaatacat attagggtta |
| 54001 | tctgcggatt tataatttga gtgatgcatt cgactcaaca taaataattc tagaggagac |
| 54061 | gatctactat caaattcgga tcgtaaatct gtttctaaag aacggagaat atctatacat |
| 54121 | acctgattag aattcatccg tccttcagac aacatctcag acagtctggt cttgtatgtc |
| 54181 | ttaatcatat tcttatgaaa cttggaaaca tctcttctag tttcactagt acctttatta |
| 54241 | attctctcag gtacagattt tgaattcgac gatgccgagt atttcatcgt tgtatatttc |
| 54301 | ttcttcgatt gcataatcaa attcttatat accgcctcaa actctatttt aaaattatca |
| 54361 | aacaatactc tactattaat cagtcgttct aactcctttg ctatttctat ggacttatct |
| 54421 | acatcttgac tgtctatctc tgtaaacacg gagtcggtat ctccatacac gctacgaaaa |
| 54481 | cgaaatctat aatctatagg caacgatgtt ttcacaatcg gattaatatc tctatcgtcc |
| 54541 | atataaaatg gattacttaa tgtattggca aaccgtaaca taccgttgga taactctgct |
| 54601 | ccatttagta ccgattctag atacaagatc attctacgtc ctatggatgt gcaactctta |
| 54661 | gccgaagcgt atgagtatag agcactattt ctaaatccca tcagaccata tactgagttg |
| 54721 | gctactatct tgtacgtata ttgcatggaa tcatagatgg ccttttcagt tgaactggta |
| 54781 | gcctgtttta acatcttttt atatctggct ctctctgcca aaaatgttct taatagtcta |
| 54841 | ggaatggttc cttctattga tctatcgaaa attgctattt cagagatgag gttcggtagt |
| 54901 | ctaggttcac aatgaaccgt aatatatcta ggaggtggat atttctgaag caagagctga |
| 54961 | ttatttattt cttcttccaa tctattggta ctaacaacga caccgactaa tgtttccgga |
| 55021 | gatagatttc caaagataca cacattagga tacagactgt tataatcaaa gattaataca |
| 55081 | ttattactaa acattttttg ttttggagca aataccttac cgccttcata aggaaacttt |
| 55141 | tgttttgttt ctgatctgac taagatagtt ttagtttcca acaatagctt taacagtgga |
| 55201 | cccttgatga ctgtactcgc tctatattcg aataccatgg attgaggaag cacatatgtt |
| 55261 | gacgcaccag cgtctgtttt tgtttctact ccataatact cccacaaata ctgacacaaa |
| 55321 | caagcatcat gaatacagta tctagccata tctaaagcta tgtttagatt ataatcctta |
| 55381 | tacatctgag ctaaatcaat gtcatccttt ccgaaagata atttatatgt atcattaggt |
| 55441 | aaagtaggac atgatagtac gactttaaat ccattttccc aaatatcttt acgaattact |
| 55501 | ttacatataa tatcctcatc aacagtcaca taattacctg ttgttaaaac ctttgcaaat |
| 55561 | gtatcggctt tgcctttcgc gtccgtagta tcgtcaccga tgaacgtcat ttctctaact |
| 55621 | cctctattta atactttacc catgcaactg aacgcgttct tggatataga atccaatttg |
| 55681 | tacgaatcca atttttcaga tttttgaatg aatgaatata gatcgaaaaa tatagttcca |
| 55741 | ttattgttat taacgtgaaa cgtagtattg gccatgccgc atactccctt atgactagac |
| 55801 | tgatttctct cataaataca gagatgtaca gcttcctttt tgtctggaga tctaaagata |
| 55861 | atcttctctc ctgttaataa ctctagacga ttagtaatat atctcagatc aaagttatgt |
| 55921 | ccgttaaagg taacgacgta gtcgaacgtt agttccaaca attgtttagc tattcgtaac |
| 55981 | aaaactattt cagaacatag aactagttct cgttcgtaat ccatttccat tagcgactgt |
| 56041 | atcctcaaac atcctctatc gacggcttct tgtatttcct gttccgttaa catctcttca |
| 56101 | ttaatgagcg taaacagtaa tcgtttacca cttaaatcga tataacagta acttgtatgc |
| 56161 | gagattgggt taataaatac agaaggaaac ttcttatcga agtgacactc tatatctaga |
| 56221 | aataagtacg atcttgggat atcgaatcta ggtatttctt tagcgaaaca gttacgtgga |
| 56281 | tcgtcacaat gataacatcc attgttaatc tttgtcaaat attgctcgtc caacgagtaa |
| 56341 | catccgtctg gagatatccc gttagaaata taaaaccaac taatattgag aaattcatcc |
| 56401 | atggtggcat tttgtatgct gcgtttcttt ggctcttcta tcaaccacat atctgcgacg |
| 56461 | gagcattttc tatctttaat atctagatta taacttattg tctcgtcaat gtctatagtt |
| 56521 | ctcatctttc ccatcggcct cgcattaaat ggaggaggag ataatgactg atatatttcg |
| 56581 | tccgtcacta cgtaataaaa gtaatgagga aatcgtataa atactgtctc gccatttcga |
| 56641 | catctggatt tcagatataa aaatctgttt tcaccgtgac tttcaaacca attaatacac |
| 56701 | ctaacatcca t......... .......... .......... .......... .......... |

1. A set of primers and optional probe(s) for detecting and/or identifying the presence of a monkeypox (MPOX) Clade II virus in a sample, the set comprising:
at least one primer pair and optional probe that selectively hybridizes to the OPG153 gene of MPOX Clade II, the OPG183 intergenic region of MPOX Clade II or a combination thereof, of MPOX Clade II virus,
wherein said set of primers and optional probe(s) selectively hybridizes to one or more conserved regions of nucleic acid from MPOX Clade II viruses.

2. The set of claim 1, wherein each primer and optional probe has less than 90%, preferably less than 85% sequence identity to an analogous region, when present, in MPOX Clade I or non-variola orthopoxviruses.

3. The set of claim 1, wherein the primers and optional probe(s) do not selectively hybridize with, detect, and/or identify the presence of MPOX Clade I viruses or non-variola orthopoxviruses.

4. The set of claim 1, further comprising an additional primer pair and optional probe for detecting and/or identifying the presence of one or more non-variola orthopoxviruses, wherein said additional primer pair and optional probe comprises at least one primer pair and optional probe that selectively hybridizes to the DNA polymerase (E9L) gene of non-variola orthopoxvirus.

5. The set of claim 4, wherein the additional primer pair and optional probe for identifying one or more non-variola orthopoxviruses selectively hybridizes to one or more conserved regions of nucleic acid from non-variola orthopoxviruses.

6. The set of claim 1, further comprising an additional primer pair and optional probe for detecting and/or identifying the presence of one or more MPOX Clade I viruses, wherein said additional primer pair and optional probe comprises at least one primer pair and optional probe that selectively hybridizes to one or more MPOX Clade I viruses.

7. The set of claim 1, further comprising one or more additional primer pairs and optional probe(s) for detecting and/or identifying the presence of one or more of herpes simplex virus (HSV), varicella zoster virus (VZV), and syphilis.

8. The set of claim 4, wherein the set comprises:
the at least one primer pair and probe that selectively hybridizes to the OPG153 gene of MPOX Clade II,
the at least one primer pair and probe that selectively hybridizes to the intergenic region of MPOX Clade II, and
the at least one primer pair and probe that selectively hybridizes to the DNA polymerase (E9L) gene of non-variola orthopoxvirus.

9. The set of claim 1, wherein, when present, the the primer and optional probe that selectively hybridizes to the OPG153 gene of MPOX Clade II comprise a sequence that is identical or complementary to at least 15 contiguous nucleotides of one or more of SEQ ID NOs: 1, 25, 26, 27, and 28;
the primers and optional probe that selectively hybridizes to the OPG183 intergenic region of MPOX Clade II comprise a sequence that is identical or complementary to at least 15 contiguous nucleotides of one or more of SEQ ID NOs:2, 29, 30, 31, and 32;
the primers and optional probe that selectively hybridizes to the DNA polymerase (E9L) gene of non-variola orthopoxvirus comprise a sequence that is identical or complementary to at least 15 contiguous nucleotides of one or more of SEQ ID NO: 3, 33, 34, 35, and 36.

10. The set of claim 1, wherein at least one of the primers and optional probe(s) comprises a detectable label, optionally wherein at least one probe, optionally each probe, of the set comprises a fluorescent dye and a quencher molecule.

11. The set of claim 1, wherein the set further comprises a primer pair that selectively hybridizes to an exogenous control and/or an endogenous control, wherein the exogenous control is a sample processing control, and wherein the endogenous control is a sample adequacy control.

12. The set of claim 1, wherein the set is contained within one or more cartridge(s).

13. The set of claim 12, wherein the set is contained in one cartridge.

14. A cartridge for detecting and/or identifying monkeypox (MPOX) Clade II in a sample, the cartridge comprising:
a cartridge body comprising a plurality of chambers therein, wherein the plurality of chambers includes:
i) a sample chamber having at least a fluid outlet in fluidic communication with another chamber of the plurality; and
ii) an optional lysis chamber in fluidic communication with the sample chamber, optionally wherein the sample chamber and lysis chamber are the same;
a reaction vessel fluidically coupled to the plurality of chambers of the cartridge body and configured for: i) amplification of nucleic acid and ii) detection and identification of one or more amplification products via real-time PCR, melt curve analysis, or a combination thereof;
a filter disposed in a fluidic path between the lysis chamber and the reaction vessel, and
a set of primers and/or probes according to claim 1, for identifying the presence of MPOX Clade II.

15. The cartridge of claim 14, wherein the cartridge is a Clinical Laboratory Improvement Amendments (CLIA)-compliant cartridge.

16. A method for detecting and identifying monkeypox (MPOX) Clade II in a sample, the method comprising:
a) contacting nucleic acid from the sample with a set of primers and optional probe(s) according to claim 1;
b) subjecting the nucleic acid, primers and optional probe(s) to amplification conditions, optionally followed by a melt curve assay;
c) detecting the presence of any amplification product(s) via real-time PCR, melt curve analysis, or a combination thereof, and
d) identifying the presence of a MPOX Clade II virus in the sample or determining that no MPOX Clade II virus detectable using the set of primers and/or probes is present, based on detection of the amplification product(s) or lack thereof, respectively.

17. The method of claim 16, wherein d) comprises differentially identifying the presence of a MPOX Clade II and/or non-variola orthopoxvirus in the sample, or determining that no MPOX Clade II and non-variola orthopoxvirus are detectable using the set of primers and/or probes present, based on detection of the amplification product(s) or lack thereof, respectively.

18. The method of claim 16, wherein:
a) said contacting nucleic acid from the sample with the set of primers and/or probes comprises:
placing the sample in a cartridge comprising a cartridge body having a plurality of chambers in fluidic communication, a reaction vessel having one or more reaction chambers and configured for amplification of the nucleic acid and detection of one or more amplification products, a fluidic path between the plurality of chambers and the reaction vessel, and a filter in the fluidic path; and
if the sample comprises cells, lysing cells in the sample with one or more lysis reagents present within at least one of the plurality of chambers;
b) said subjecting the nucleic acid, primers and/or probes to amplification conditions comprises amplifying the nucleic acid with primers and/or probes present in solution within the reaction vessel.

19. The method of claim 16, wherein the sample is a skin sample, a lesion swab sample, a vesicular lesion fluid sample, a pustular lesion fluid sample, a rectal sample, a nasal aspirate sample, a nasal wash sample, a nasal swab sample, a nasopharyngeal swab sample, a saliva sample, an oropharyngeal swab sample, a throat swab sample, a bronchoalveolar lavage sample, a bronchial aspirate sample, a bronchial wash sample, an endotracheal aspirate sample, an endotracheal wash sample, a tracheal aspirate sample, a nasal secretion sample, a mucus sample, a sputum sample, a plasma sample, a whole blood sample, or a combination thereof.

20. The method of claim 16, wherein the method is carried out to distinguish between a virulent pathogen and a less virulent pathogen.

21. The set of claim 12, wherein the cartridge facilitates detection of a MPOX Clade II virus within the sample within 60 minutes, within 45 minutes, or within 30 minutes of collecting the sample from the subject.

22. The set of claim 12, wherein the cartridge facilitates detection of a MPOX Clade II virus within the sample within 60 minutes, within 45 minutes, within 30 minutes, or within 20 minutes from the time the sample is placed in a cartridge.

23. The set of claim 21, wherein the reaction vessel comprises up to 4 reaction chambers.

24. The set of claim 21, wherein the reaction vessel comprises one reaction chamber.

25. A system for detecting monkeypox (MPOX) Clade II in a biological sample, the system comprising:
a module having a receiving bay for receiving the cartridge of claim 14, wherein the module includes one or more mechanisms within the receiving bay for manipulating a fluid sample within the cartridge, and an instrument that interfaces with the reaction vessel; and
a memory having programmable instructions recorded thereon, that are specially configured to operate the module according to a monkepox Clade II assay protocol to determine nucleic acid sequence characteristics of MPOX Clade II.


SEQUENCE LISTING (122 sequences)

SEQ ID NO: 1 - DNA, 3697 bp, Monkeypox virus
  source 1..3697: mol_type = genomic DNA
ttaaaccgtcttttctggtataatattgtttagtagatactcatcaagataagctaattcactaaacatattatcggattcggtattgttactcgagaatagagttcgttatgctcctgatattcggaaatctgtggagtttcaggttttggtggaagtgtaactgctacttggtgggatactgaaggatatttcagagagttgtggatgttcgggttcgacatccaccgatggtgtcacgccactaatcggttcggtaacgtctgtggatggaggtgctacttctacagaacctgtagcctcagttgtcaacggagatacatattcaatgcgcggaaatgtataatttggtaatggtttctcatgtggatcttaagaagaagaggtaagatatctacgaaagataccgatcacgtttctagttctcttttgtagaactttaactttttctttctcagcatctagttgatattccgacctcttcacgtttcgcatgggttacctccgcagtttttacaagcgatttcacgttccagatcacgttcagccttcatacgtctctccctctctctatcgagtttatcagagcagtctttctgaaggcgatcgaactccataaatttctccaacgctttgattgtttccatagatttccgaagtttagcttctaggacggcgattctttttttttttttttttttttttttcgaattcacggggtacaaccgtttccattaccaccatctctatgtttcttttctagatcggcaatctttctcaatctttctcaacatttcatccccataccttttcattcctcgagtctattgtcgtcgaaatatcgttccagctccttttcgacctcaataactttagcacgttgtttcatcaagctctctcttgtagtactatcatttttatctgattccctgacacgtttaagatcttcatgtaattgagtcagctcttgacgcaatctcttaactaacttcctctcttgcttcttcgtcatagtacttacaatcactatgggatccattgttaccacgtctgtactcgacgagctcacgtttaagagattcaatttccagtttgtatcggtccatgtctccattgctacaccaccattagatttacaggctgctagttgtcgttcgagatcagaaatacgtgttttcttggaatggatttcgtcgatgtacttgtcatgattggcatcgaaacacttattaagttctttttttcaattctacgattttatttctttcgcgagtcaattccctcctgtagtaactatcagttttgtcagattcacgctctctacgtagactttcttgtaagttactaatttgttccctggcattaccgagttcagttttatatgccgaatagagttctgattcatcctttgagaagatctctagcgatcgttcaagatccctgattctagtctttagcctatttacctcctcagaagatgctccgttaccgtttttacaatcgttaagatgtctatcaagatccatgattctatctcttttccatatcagcattgatttcattattacgttcgcagtcgttcaactgtatttcaagatctgagattctagattgtaatctctgtagcatttccacggcattcactcagttgtctttcaagatctgagattctagattggagtctgctaatctctgtaagatttcctcctccgctctcgatgcagtcggtcaacttattctctagttctctaatacgcgaacgcagtgcatcaacttcttgtgtgtcttcttgattgcgtgtgcattcatcgagtctagattcgagatctctaacgtgacgtcgttcttcctcaagttctctgtgtactacagaaagcgtgtccctatcttgttgatatttagcaatttctgattctagagtactgattctactcacgtatgtactaatagttgtcttagccttatcaagatcctccttgtatttgtcacattccttgatatccatacgaagtctggacagttcccattcgacattacgacgtttatcgatttcagctcggagatcgtcgtcgcgttgttttagccacatacgactaagttcaagttctcgttgacaagatccatctacttttccatccctaatagtatccagttccttttctagttctgaccgcatttctcgttccatatcaagagattctctcaattctcgtatagtcttcttatcaatttctgatgaatctgaaccatcatctgtcccattttgttgcatatccctgagttctttgatctctgttgtaagtctgtcgattctttcggttttataaacagaatccctttccaaagtcctaatcttactgagtttatcattaagttcttcattcaattcagtgagttttctcttggcttcttccaagtctgttttaaactctccatcatttccgcattcttcctcgcatttatctaaccattcaattagtttattaataactagttggtaatcagcgattcctatagccgttcttgtatttgtgggaacataattaggatcttctaatggattgtatggcttgatagcatcatctttatcattattaggtgggggatggacaaccttaattggttggtcctccttatctcctccagtagcatgtggttcttcaataccagtattagtaataggcttagacaaatgcttgtcgtacgcgggcacttcctcatccatcaagtatttataatcgggttctgtttcagaatattcttttctaagagacgcgacttcaggagttagtagaagaactctgtttctgtatctatcaacgctggaatcgatactcaagttaaggatagcgaatacctcatcgtcatcatccgtatcttctgaaacgccatcatatgacatttcatgaagtctaacgtattgataaacagaatcagatttagtattaaacagatccttgacctttttagtaaatgcatatgtatattttagatctccagatttcataatatgatcgcatgccttaaatgtcaatgcttccatgatatagtctggaacactaatgggtgacgaaaaagatacagcaccatatgctacgttgataaatagatctgaaccactaagtagataatgattaatgttaaggaagaggaaatattcagtatatagatatgccttagcatcatatcttgtactaaacacgctaaacagtttattgatgtgatcaatttccaacagaacaattagagcggcaggaataccaacaaacatattaccacatccgtattttctatgaatatcacatatcatattaaaaaatcttgatagaagagcgaatatctcgtctgacttaatgagatgtagttcagcagcataagtcataactgtaaatagaacatactttcctgtagtgttgattctagactccacatcaacaccattattaaaaatagttttatatacatctttaatctgctctccgttaatcgtcgaacgttctagtatacggaaacactttgatttcttatctgtagttaatgacttagtgatatcacgaagaatattacgaattacatttcttgtttttcttgagagacctgattcagaactcaactcatcgttccatagtttttctacctcagtggcgaaatctttggagtgtttggtacattttttaataaggttcgtgacctccat

SEQ ID NO: 2 - DNA, 566 bp, Monkeypox virus
  source 1..566: mol_type = genomic DNA
atggatataaagaatttactgactacatgtactattttacattactacattggctacggcatatatacctatttcgtcacttccacacgctccggtaaacgggtgtcatgtgacgagggagaatcttgataagaggcataatcaatgttgtaatccgatgtccacctggagaatttgccaaggtcagatgtagagttggtagtgataacacaaaatgtgaacactgcccacctcatacatataccgcaatccccaattattctaatagatgtcatcaatgtagaaaatgcccaacaggatcatttgataaggtaaagtgtaccggaacacagaacaaatgttcgtgtcatcctggttggtatacgctactgattcttcacagactgaagattgtcgagatttgtgtaccaaaaaaggagatgtccatgcggatactttggtggaatagatgaaggaaatcctatttgtaaatcgtgttgtgttggtgaatattgcgactacctacgtaattatagacttgatccatttcctccatgcaaactatctatctaaatgtaattaattat

SEQ ID NO: 3 - DNA, 3021 bp, Monkeypox virus
  source 1..3021: mol_type = genomic DNA
ttatgcttcgtaaaatgtaggtcttgaaccaaacattctttgaaaaaatgagatgcataaaactttattatccaatagattaactatttcagacgtcaatcgtttaaagtaaacttcgtaaaatattctttgattgctgccgagtttaaaacttctatcgataattgtttcatatgttttaatatttacaagttttttggtccatggtacattagctggacagatatatgcaaaataatatcgttctccaagttctatagtctctggattgtttttattatattcagtaaccaaatacatattagggttatctgcggatttataatttgagtgatgcattcgactcaacataaataattctagaggagacgatctactatcaaattcggatcgtaaatctgtttctaaagaacggagaatatctatacatacctgattagaattcatccgtccttcagacaacatctcagacagtctggtcttgtatgtcttaatcatattcttatgaaacttggaaacatctcttctagtttcactagtacctttattaattctctcaggtacagattttgaattcgacgatgccgagtatttcatcgttgtatatttcttcttcgattgcataatcaaattcttatataccgcctcaaactctattttaaaattatcaaacaatactctactattaatcagtcgttctaactcctttgctatttctatggacttatctacatcttgactgtctatctctgtaaacacggagtcggtatctccatacacgctacgaaaacgaaatctataatctataggcaacgatgttttcacaatcggattaatatctctatcgtccatataaaatggattacttaatgtattggcaaaccgtaacataccgttggataactctgctccatttagtaccgattctagatacaagatcattctacgtcctatggatgtgcaactcttagccgaagcgtatgagtatagagcactatttctaaatcccatcagaccatatactgagttggctactatcttgtacgtatattgcatggaatcatagatggccttttcagttgaactggtagcctgttttaacatctttttatatctggctctctctgccaaaaatgttcttaatagtctaggaatggttccttctattgatctatcgaaaattgctatttcagagatgaggttcggtagtctaggttcacaatgaaccgtaatatatctaggaggtggatatttctgaagcaagagctgattatttatttcttcttccaatctattggtactaacaacgacaccgactaatgtttccggagatagatttccaaagatacacacattaggatacagactgttataatcaaagattaatacattattactaaacattttttgttttggagcaaataccttaccgccttcataaggaaacttttgttttgtttctgatctgactaagatagttttagtttccaacaatagctttaacagtggacccttgatgactgtactcgctctatattcgaataccatggattgaggaagcacatatgttgacgcaccagcgtctgtttttgtttctactccataatactcccacaaatactgacacaaacaagcatcatgaatacagtatctagccatatctaaagctatgtttagattataatccttatacatctgagctaaatcaatgtcatcctttccgaaagataatttatatgtatcattaggtaaagtaggacatgatagtacgactttaaatccattttcccaaatatctttacgaattactttacatataatatcctcatcaacagtcacataattacctgttgttaaaacctttgcaaatgtatcggctttgcctttcgcgtccgtagtatcgtcaccgatgaacgtcatttctctaactcctctatttaatactttacccatgcaactgaacgcgttcttggatatagaatccaatttgtacgaatccaatttttcagatttttgaatgaatgaatatagatcgaaaaatatagttccattattgttattaacgtgaaacgtagtattggccatgccgcatactcccttatgactagactgatttctctcataaatacagagatgtacagcttcctttttgtctggagatctaaagataatcttctctcctgttaataactctagacgattagtaatatatctcagatcaaagttatgtccgttaaaggtaacgacgtagtcgaacgttagttccaacaattgtttagctattcgtaacaaaactatttcagaacatagaactagttctcgttcgtaatccatttccattagcgactgtatcctcaaacatcctctatcgacggcttcttgtatttcctgttccgttaacatctcttcattaatgagcgtaaacagtaatcgtttaccacttaaatcgatataacagtaacttgtatgcgagattgggttaataaatacagaaggaaacttcttatcgaagtgacactctatatctagaaataagtacgatcttgggatatcgaatctaggtatttctttagcgaaacagttacgtggatcgtcacaatgataacatccattgttaatctttgtcaaatattgctcgtccaacgagtaacatccgtctggagatatcccgttagaaatataaaaccaactaatattgagaaattcatccatggtggcattttgtatgctgcgtttctttggctcttctatcaaccacatatctgcgacggagcattttctatctttaatatctagattataacttattgtctcgtcaatgtctatagttctcatctttcccatcggcctcgcattaaatggaggaggagataatgactgatatatttcgtccgtcactacgtaataaaagtaatgaggaaatcgtataaatactgtctcgccatttcgacatctggatttcagatataaaaatctgttttcaccgtgactttcaaaccaattaatacacctaacatccat

SEQ ID NO: 4 - DNA, 20 bp, synthetic construct
  source 1..20: mol_type = other DNA
tagtgagttcggcgacaaag

SEQ ID NO: 5 - DNA, 22 bp, synthetic construct
  source 1..22: mol_type = other DNA
gtatcgcatctctcgggtattc

SEQ ID NO: 6 - DNA, 25 bp, synthetic construct
  source 1..25: mol_type = other DNA
accggtaatcttgtcgaggaggaca

SEQ ID NO: 7 - DNA, 25 bp, synthetic construct
  source 1..25: mol_type = other DNA
catctattatagcatcagcatcaga

SEQ ID NO: 8 - DNA, 23 bp, synthetic construct
  source 1..23: mol_type = other DNA
gatactcctcctcgttggtctac

SEQ ID NO: 9 - DNA, 25 bp, synthetic construct
  source 1..25: mol_type = other DNA
tgtaggccgtgtatcagcatccatt

SEQ ID NO: 10 - DNA, 24 bp, synthetic construct
  source 1..24: mol_type = other DNA
tgtctacctggatacagaaagcaa

SEQ ID NO: 11 - DNA, 24 bp, synthetic construct
  source 1..24: mol_type = other DNA
ggcatctccgtttaatacattgat

SEQ ID NO: 12 - DNA, 31 bp, synthetic construct
  source 1..31: mol_type = other DNA
cccatatatgctaaatggtaccggtaccgga

SEQ ID NO: 13 - DNA, 20 bp, synthetic construct
  source 1..20: mol_type = other DNA
cacaccgtctcttccacaga

SEQ ID NO: 14 - DNA, 23 bp, synthetic construct
  source 1..23: mol_type = other DNA
gatacaggttaatttccacatcg

SEQ ID NO: 15 - DNA, 26 bp, synthetic construct
  source 1..26: mol_type = other DNA
aacccgtcgtaaccagcaatacattt

SEQ ID NO: 16 - DNA, 19 bp, synthetic construct
  source 1..19: mol_type = other DNA
ctatcaaagagaaggccaa

SEQ ID NO: 17 - DNA, 22 bp, synthetic construct
  source 1..22: mol_type = other DNA
attctcataatttcctttccat

SEQ ID NO: 18 - DNA, 27 bp, synthetic construct
  source 1..27: mol_type = other DNA
acaatctcctatccagaggtggtaatc

SEQ ID NO: 19 - DNA, 24 bp, synthetic construct
  source 1..24: mol_type = other DNA
taatacttcgattgctcatccagg

SEQ ID NO: 20 - DNA, 26 bp, synthetic construct
  source 1..26: mol_type = other DNA
acttctcacaaatggatttgaaaatc

SEQ ID NO: 21 - DNA, 20 bp, synthetic construct
  source 1..20: mol_type = other DNA
tcctttacgtcataaatcat

SEQ ID NO: 22 - DNA, 23 bp, synthetic construct
  source 1..23: mol_type = other DNA
tcaactgaaaaggccatctatga

SEQ ID NO: 23 - DNA, 28 bp, synthetic construct
  source 1..28: mol_type = other DNA
gagtatagagcactatttctaaatccca

SEQ ID NO: 24 - DNA, 32 bp, synthetic construct
  source 1..32: mol_type = other DNA
ccatgcaatatacgtacaagatagtagccaac

SEQ ID NO: 25 - DNA, 25 bp, synthetic construct
  source 1..25: mol_type = other DNA
gttcaagatccctgattctagtctt

SEQ ID NO: 26 - DNA, 25 bp, synthetic construct
  source 1..25: mol_type = other DNA
ggatcttgatagacatcttaacgat

SEQ ID NO: 27 - DNA, 25 bp, synthetic construct
  source 1..25: mol_type = other DNA
ctcagaagatgctccgttaccgttt

SEQ ID NO: 28 - DNA, 95 bp, synthetic construct
  source 1..95: mol_type = other DNA
gttcaagatccctgattctagtctttagcctatttacctcctcagaagatgctccgttaccgtttttacaatcgttaagatgtctatcaagatcc

SEQ ID NO: 29 - DNA, 25 bp, synthetic construct
  source 1..25: mol_type = other DNA
tatacctatttcgtcacttccacac

SEQ ID NO: 30 - DNA, 25 bp, synthetic construct
  source 1..25: mol_type = other DNA
ggattacaacattgattatgcctct

SEQ ID NO: 31 - DNA, 25 bp, synthetic construct
  source 1..25: mol_type = other DNA
agattctccctcgtcacatgacacc

SEQ ID NO: 32 - DNA, 93 bp, synthetic construct
  source 1..93: mol_type = other DNA
tatacctatttcgtcacttccacacgctccggtaaacgggtgtcatgtgacgagggagaatcttgataagaggcataatcaatgttgtaatcc

SEQ ID NO: 33 - DNA, 23 bp, synthetic construct
  source 1..23: mol_type = other DNA
tcaactgaaaaggccatctatga

SEQ ID NO: 34 - DNA, 28 bp, synthetic construct
  source 1..28: mol_type = other DNA
gagtatagagcactatttctaaatccca

SEQ ID NO: 35 - DNA, 32 bp, synthetic construct; identical to SEQ ID NO: 24
  source 1..32: mol_type = other DNA

SEQ ID NO: 36 - DNA, 101 bp, synthetic construct
  source 1..101: mol_type = other DNA
gagtatagagcactatttctaaatcccatcagaccatatactgagttggctactatcttgtacgtatattgcatggaatcatagatggccttttcagttga

SEQ ID NO: 37 - DNA, 25 bp, synthetic construct
  source 1..25: mol_type = other DNA
gttcaagatccctgattctagtctt

SEQ ID NO: 38 - DNA, 25 bp, synthetic construct
  source 1..25: mol_type = other DNA
ttcaagatccctgattctagtcttt

SEQ ID NO: 39 - DNA, 25 bp, synthetic construct
  source 1..25: mol_type = other DNA
tcaagatccctgattctagtcttta

SEQ ID NO: 40 - DNA, 25 bp, synthetic construct
  source 1..25: mol_type = other DNA
caagatccctgattctagtctttag

SEQ ID NO: 41 - DNA, 25 bp, synthetic construct
  source 1..25: mol_type = other DNA
aagatccctgattctagtctttagc

SEQ ID NO: 42 - DNA, 25 bp, synthetic construct
  source 1..25: mol_type = other DNA
ctcagaagatgctccgttaccgttt

SEQ ID NO: 43 - DNA, 25 bp, synthetic construct
  source 1..25: mol_type = other DNA
tcagaagatgctccgttaccgtttt

SEQ ID NO: 44 - DNA, 25 bp, synthetic construct
  source 1..25: mol_type = other DNA
cagaagatgctccgttaccgttttt

SEQ ID NO: 45 - DNA, 25 bp, synthetic construct
  source 1..25: mol_type = other DNA
agaagatgctccgttaccgttttta

SEQ ID NO: 46 - DNA, 25 bp, synthetic construct
  source 1..25: mol_type = other DNA
atcgttaagatgtctatcaagatcc

SEQ ID NO: 47 - DNA, 25 bp, synthetic construct
  source 1..25: mol_type = other DNA
tcgttaagatgtctatcaagatcca

SEQ ID NO: 48 - DNA, 25 bp, synthetic construct
  source 1..25: mol_type = other DNA
cgttaagatgtctatcaagatccat

SEQ ID NO: 49 - DNA, 25 bp, synthetic construct
  source 1..25: mol_type = other DNA
gttaagatgtctatcaagatccatg

SEQ ID NO: 50 - DNA, 25 bp, synthetic construct
  source 1..25: mol_type = other DNA
ttaagatgtctatcaagatccatga

SEQ ID NO: 51 - DNA, 103 bp, unidentified
  source 1..103: mol_type = unassigned DNA
gttcaagatccctgattctagtctttagcctatttacctcctcagaagatgctccgttaccgtttttacaatcgttaagatgtctatcaagatccatgattct

SEQ ID NO: 52 - DNA, 103 bp, unidentified
  source 1..103: mol_type = unassigned DNA
gttcaagatccctgattctagtctttagcctatttacctccttagaagatgctccgttaccgtttttacaatcgttaagatgtctatcaagatccatgattct

SEQ ID NO: 53 - DNA, 103 bp, unidentified
  source 1..103: mol_type = unassigned DNA
gttcaagatccctgattctagtctttagcctatttaccttctcagaagatgctccgttaccgtttttacaatcgttaagatgtctatcaagatccatgattct

SEQ ID NO: 54 - DNA, 102 bp, unidentified
  source 1..102: mol_type = unassigned DNA
gttcaagatccctgattctagtctttagcctatttacctcctcagaagatgctccgttaccgtttttacaatcgttaagatgtctatcaagatccatgattc

SEQ ID NO: 55 - DNA, 25 bp, synthetic construct
  source 1..25: mol_type = other DNA
gttcaagatccctgattctagtctt

SEQ ID NO: 56 - DNA, 25 bp, synthetic construct
  source 1..25: mol_type = other DNA
ttcaagatccctgattctagtcttt

SEQ ID NO: 57 - DNA, 25 bp, synthetic construct
  source 1..25: mol_type = other DNA
tcaagatccctgattctagtcttta

SEQ ID NO: 58 - DNA, 25 bp, synthetic construct
  source 1..25: mol_type = other DNA
caagatccctgattctagtctttag

SEQ ID NO: 59 - DNA, 25 bp, synthetic construct
  source 1..25: mol_type = other DNA
aagatccctgattctagtctttagc

SEQ ID NO: 60 - DNA, 25 bp, synthetic construct
  source 1..25: mol_type = other DNA
ctcagaagatgctccgttaccgttt

SEQ ID NO: 61 - DNA, 25 bp, synthetic construct
  source 1..25: mol_type = other DNA
tcagaagatgctccgttaccgtttt

SEQ ID NO: 62 - DNA, 25 bp, synthetic construct
  source 1..25: mol_type = other DNA
cagaagatgctccgttaccgttttt

SEQ ID NO: 63 - DNA, 25 bp, synthetic construct
  source 1..25: mol_type = other DNA
agaagatgctccgttaccgttttta

SEQ ID NO: 64 - DNA, 25 bp, synthetic construct
  source 1..25: mol_type = other DNA
atcgttaagatgtctatcaagatcc

SEQ ID NO: 65 - DNA, 25 bp, synthetic construct
  source 1..25: mol_type = other DNA
tcgttaagatgtctatcaagatcca

SEQ ID NO: 66 - DNA, 25 bp, synthetic construct
  source 1..25: mol_type = other DNA
cgttaagatgtctatcaagatccat

SEQ ID NO: 67 - DNA, 25 bp, synthetic construct
  source 1..25: mol_type = other DNA
gttaagatgtctatcaagatccatg

SEQ ID NO: 68 - DNA, 25 bp, synthetic construct
  source 1..25: mol_type = other DNA
ttaagatgtctatcaagatccatga

SEQ ID NO: 69 - DNA, 103 bp, unidentified; identical to SEQ ID NO: 51
  source 1..103: mol_type = unassigned DNA

SEQ ID NO: 70 - DNA, 106 bp, unidentified
  source 1..106: mol_type = unassigned DNA
gttcaagatccctgattctagtctttagcctatttacctcctcagaagatgatccgttaccgttgcgtttacactcgttaagctgtctatcaagatccatgattct

SEQ ID NO: 71 - DNA, 106 bp, Taterapox virus
  source 1..106: mol_type = genomic DNA
gttcaagatccctgattctagtctttagcctatttacctcctcagaagatgttccgttaccgttgcgtttacactcgttaagctgtctatcaagatccatgattct

SEQ ID NO: 72 - DNA, 108 bp, Cowpox virus
  source 1..108: mol_type = genomic DNA
gttcaagatccctgattctagtctttagcctatttacctcctcagaagatgtttccgttaccgtcgcgtttacatttcgttaagctgtctatcaagatccatgattct

SEQ ID NO: 73 - DNA, 109 bp, Vaccinia virus
  source 1..109: mol_type = genomic DNA
gttttaagattcctgattctagtctttagcctatttacctcctcagaagatgttccgttaccgttgcgtttacacctcgttaagcctgtctatcaagatccatgattct

SEQ ID NO: 74 - DNA, 106 bp, Variola virus
  source 1..106: mol_type = genomic DNA
gtttaagatcactaattctagtctttagcctatttacctcctcagaagatgttccgttaccgttgcgtttacactcgttaagctgtctatcaagatccatgattct

SEQ ID NO: 75 - DNA, 85 bp, Camelpox virus
  source 1..85: mol_type = genomic DNA
ctttaaacctatttacctcctcagaagatgttccgttaccgttgcgtttacactcgttaagttgtctatcaagatccatgattct

SEQ ID NO: 76 - DNA, 104 bp, Akhmeta virus
  source 1..104: mol_type = genomic DNA
gttctagatctctgatacggactctttagcctatctacctcttctgaagatgatccaccgttgcgtttacactcgttaagttgtctatccagctccatgattct

SEQ ID NO: 77 - DNA, 85 bp, Ectromelia virus
  source 1..85: mol_type = genomic DNA
tctttagcctatctatttcctcagaagatgatccattaccgttacgtctacactcattaagttgcttatccagatccatgattct

SEQ ID NO: 78 - DNA, 25 bp, synthetic construct
  source 1..25: mol_type = other DNA
tatatacctatttcgtcacttccac

SEQ ID NO: 79 - DNA, 25 bp, synthetic construct
  source 1..25: mol_type = other DNA
atatacctatttcgtcacttccaca

SEQ ID NO: 80 - DNA, 25 bp, synthetic construct
  source 1..25: mol_type = other DNA
tatacctatttcgtcacttccacac

SEQ ID NO: 81 - DNA, 25 bp, synthetic construct
  source 1..25: mol_type = other DNA
ctccggtaaacgggtgtcatgtgac

SEQ ID NO: 82 - DNA, 25 bp, synthetic construct
  source 1..25: mol_type = other DNA
cggtaaacgggtgtcatgtgacgag

SEQ ID NO: 83 - DNA, 25 bp, synthetic construct
  source 1..25: mol_type = other DNA
ggtgtcatgtgacgagggagaatct

SEQ ID NO: 84 - DNA, 25 bp, synthetic construct
  source 1..25: mol_type = other DNA
gtgtcatgtgacgagggagaatctt

SEQ ID NO: 85 - DNA, 25 bp, synthetic construct
  source 1..25: mol_type = other DNA
tgtcatgtgacgagggagaatcttg

SEQ ID NO: 86 - DNA, 25 bp, synthetic construct
  source 1..25: mol_type = other DNA
aagaggcataatcaatgttgtaatc

SEQ ID NO: 87 - DNA, 25 bp, synthetic construct
  source 1..25: mol_type = other DNA
agaggcataatcaatgttgtaatcc

SEQ ID NO: 88 - DNA, 25 bp, synthetic construct
  source 1..25: mol_type = other DNA
gcataatcaatgttgtaatccgatg

SEQ ID NO: 89 - DNA, 25 bp, synthetic construct
  source 1..25: mol_type = other DNA
cataatcaatgttgtaatccgatgt

SEQ ID NO: 90 - DNA, 25 bp, synthetic construct
  source 1..25: mol_type = other DNA
ataatcaatgttgtaatccgatgtc

SEQ ID NO: 91 - DNA, 109 bp, unidentified
  source 1..109: mol_type = unassigned DNA
ctacggcatatatacctatttcgtcacttccacacgctccggtaaacgggtgtcatgtgacgagggagaatcttgataagaggcataatcaatgttgtaatccgatgtc

SEQ ID NO: 92 - DNA, 109 bp, unidentified
  source 1..109: mol_type = unassigned DNA
ctacggcatatatacctattttgtcacttccacacgctccggtaaacgggtgtcatgtgacgagggagaatcttgataagaggcataatcaatgttgtaatccgatgtc

SEQ ID NO: 93 - DNA, 107 bp, unidentified
  source 1..107: mol_type = unassigned DNA
ctacggtatatacctacttcgtcacttccacacgctccagaaaacgggtgtcatgtgacgagggagaatcttgataagaggcataatcaatgttgtaatccggtgtc

SEQ ID NO: 94 - DNA, 107 bp, unidentified
  source 1..107: mol_type = unassigned DNA
ctacggtatatacctacttcgtcacttccacacgctccagtaaacgggtgtcatgtgacgagggagaatcttgataagaggcataatcaatgttgtaatccggtgtc

SEQ ID NO: 95 - DNA, 109 bp, unidentified
  source 1..109: mol_type = unassigned DNA
ctacggcatatatacctatttcgtcacttccacacgctccggtaaacgggtgtcatgtgacaagggagaatcttgataagaggcataatcaatgttgtaatccgatgtc

SEQ ID NO: 96 - DNA, 107 bp, unidentified
  source 1..107: mol_type = unassigned DNA
ctacggtatatacctatttcgtcacttccacacgctccggtaaacgggtgtcatgtgacgagggagaatcttgataagaggcataatcaatgttgtaatccgatgtc

SEQ ID NO: 97 - DNA, 107 bp, unidentified
  source 1..107: mol_type = unassigned DNA
ctacggtatatacctacttcgtcacttcaacacgctccagtaaacgggtgtcatgtgacgagggagaatcttgataagaggcataatcaatgttgtaatccggtgtc

SEQ ID NO: 98 - DNA, 109 bp, unidentified
  source 1..109: mol_type = unassigned DNA
ctacggcatatatacctattttgtcacttccacacgctccggtaaacgggtgtcatgtgacgagggagaatcttgataagaggcataatcaatgttgtaattcgatgtc

SEQ ID NO: 99 - DNA, 109 bp, unidentified
  source 1..109: mol_type = unassigned DNA
ctacggcatatatacctatttcgtcactttcacacgctccggtaaacgggtgtcatgtgacgagggagaatcttgataagaggcataatcaatgttgtaatccgatgtc

SEQ ID NO: 100 - DNA, 25 bp, synthetic construct
  source 1..25: mol_type = other DNA
tatatacctatttcgtcacttccac

SEQ ID NO: 101 - DNA, 25 bp, synthetic construct
  source 1..25: mol_type = other DNA
atatacctatttcgtcacttccaca

SEQ ID NO: 102 - DNA, 25 bp, synthetic construct
  source 1..25: mol_type = other DNA
tatacctatttcgtcacttccacac

SEQ ID NO: 103 - DNA, 25 bp, synthetic construct
  source 1..25: mol_type = other DNA
ctccggtaaacgggtgtcatgtgac

SEQ ID NO: 104 - DNA, 25 bp, synthetic construct
  source 1..25: mol_type = other DNA
cggtaaacgggtgtcatgtgacgag

SEQ ID NO: 105 - DNA, 25 bp, synthetic construct
  source 1..25: mol_type = other DNA
ggtgtcatgtgacgagggagaatct

SEQ ID NO: 106 - DNA, 25 bp, synthetic construct
  source 1..25: mol_type = other DNA
gtgtcatgtgacgagggagaatctt

SEQ ID NO: 107 - DNA, 25 bp, synthetic construct
  source 1..25: mol_type = other DNA
tgtcatgtgacgagggagaatcttg

SEQ ID NO: 108 - DNA, 25 bp, synthetic construct
  source 1..25: mol_type = other DNA
aagaggcataatcaatgttgtaatc

SEQ ID NO: 109 - DNA, 25 bp, synthetic construct
  source 1..25: mol_type = other DNA
agaggcataatcaatgttgtaatcc

SEQ ID NO: 110 - DNA, 25 bp, synthetic construct
  source 1..25: mol_type = other DNA
gcataatcaatgttgtaatccgatg

SEQ ID NO: 111 - DNA, 25 bp, synthetic construct
  source 1..25: mol_type = other DNA
cataatcaatgttgtaatccgatgt

SEQ ID NO: 112 - DNA, 25 bp, synthetic construct
  source 1..25: mol_type = other DNA
ataatcaatgttgtaatccgatgtc

SEQ ID NO: 113 - DNA, 109 bp, unidentified; identical to SEQ ID NO: 91
  source 1..109: mol_type = unassigned DNA

SEQ ID NO: 114 - DNA, 102 bp, unidentified
  source 1..102: mol_type = unassigned DNA
ctacggcagatatacctacttcgtcactgccacacgctccggtaaacgggtcatgtgacgagggagaatatcttgataagaggtataatcaatgttgtaatc

SEQ ID NO: 115 - DNA, 101 bp, Ectromelia virus
  source 1..101: mol_type = genomic DNA
ctacggcagatatacctacttcgtcactgccacacgctccggtaaacgggtcatgtgacgagggagaatatcttgataaaggcataatcaatgttgtaatc

SEQ ID NO: 116 - DNA, 101 bp, Cowpox virus
  source 1..101: mol_type = genomic DNA
tacagcagatatacctacttcgtcactgccacacgctccggtaaacgggtcatgtgacgacggagaatatcttgataagacgcataatcaatgttgtaatc

SEQ ID NO: 117 - DNA, 98 bp, Camelpox virus
  source 1..98: mol_type = genomic DNA
tacggcagatatacctactccgccaccacatgctccggtaaacgggtcatgtgccgagggagaatatcttgataagactaataatcgatgttgtaatc

SEQ ID NO: 118 - DNA, 98 bp, Taterapox virus
  source 1..98: mol_type = genomic DNA
tacggcagatatacctactccgccaccacatgctccggtaaacgggtcatgtaccgagggagaatatcttgataagactcataattgatgttgtaatc

SEQ ID NO: 119 - DNA, 58 bp, Vaccinia virus
  source 1..58: mol_type = genomic DNA
aacgggtcatgtgacaagggagaatatcttgataagaggcataatcaatgttgtaatc

SEQ ID NO: 120 - DNA, 71 bp, Akhmeta virus
  source 1..71: mol_type = genomic DNA
ccacatcctcctgtagacagatcatgcaatgagggagaatatcttgatagaacgactaatcaatgttgcaa

SEQ ID NO: 121 - DNA, 103 bp, synthetic construct; identical to SEQ ID NO: 51
  source 1..103: mol_type = other DNA

SEQ ID NO: 122 - DNA, 109 bp, synthetic construct; identical to SEQ ID NO: 91
  source 1..109: mol_type = other DNA